(19) United States
(12) Patent Application Publication
(10) Pub. No.: US 2022/0344599 A1
(43) Pub. Date: Oct. 27, 2022

(54) METAL COMPLEX AND ELECTRON TRANSPORTING MATERIAL COMPRISING SAME

(71) Applicant: DYDEN CORPORATION, Kurume-shi, Fukuoka (JP)

(72) Inventors: Yumi SAKAI, Kurume-shi, Fukuoka (JP); Masataka WATANABE, Kurume-shi, Fukuoka (JP); Kentaro YAMATO, Kurume-shi, Fukuoka (JP); Chengzhou LI, Kurume-shi, Fukuoka (JP)

(21) Appl. No.: 17/765,978

(22) PCT Filed: Oct. 2, 2020
(86) PCT No.: PCT/JP2020/037496

§ 371 Date: Apr. 1, 2022

(30) Foreign Application Priority Data
Oct. 2, 2019 (JP) 2019-182141

(51) Int. Cl. H01L 51/00; C07F 1/00; C07F 1/02; C07F 9/6561

(52) U.S. Cl. CPC H01L 51/0077; C07F 1/005; C07F 1/02; C07F 9/6561; H01L 51/5076

(57) ABSTRACT

Provided is a novel metal complex that can be used as an electron transporting material. A metal complex represented by the following Formula (1) to the following Formula (3). R^{A1} to R^{A9}, R^{C1} to R^{C8}, and R^{E1} to R^{E6} are each independently a single bond, an alkylene group, an arylene group, a heteroarylene group, or a group represented by —R^{P1}—P(═O)R^{P2}—R^{P3}—, R^{B1} to R^{B9}, R^{D1} to R^{D8}, and R^{F1} to R^{F6} are each independently a hydrogen atom, an. alkyl group, an aryl group, a heteroaryl group, an alkoxy group, an aryloxy group, a heteroaryloxv group, an amino group, a cyano group, a halogen atom, or a hydroxy group, one or more selected from the group consisting of R^{B1} to R^{B9} are a phenanthrolinyl group, one or more selected from the group consisting of R^{D1} to R^{D8} are a phenanthrolinyl group, and one or more selected from the group consisting of R^{F1} to R^{F6} are a phenanthrolinyl group, M is an alkali metal or an alkaline earth metal, Z is 1 or 2, and X is O or S.

### TECHNICAL FIELD

The present invention relates to a novel alkali metal complex and an alkaline earth metal complex. The present invention also relates to an electron transporting material for organic electroluminescent elements comprising such a novel metal complex. The present invention more specifically relates to an electron. transporting material that can be formed by a wet process in production of an organic electroluminescent element having a multilayer structure, the electron transporting material having excellent electron injection properties, electron transport properties, and durability.

### BACKGROUND ART

Organic electroluminescent elements each including an emissive organic layer (organic electroluminescent layer) provided between an anode and a cathode (hereinafter, may be abbreviated as “organic EL elements”) advantageously can be driven at a lower direct-current voltage and has higher luminance and emission efficiency compared with inorganic EL elements, attracting attertion as display devices of next generation. Recently, full-color display panels have been put on the market, and research and development have been intensively conducted aiming to extension of display surface, improvement in durability, and the like.

An organic EL element is an electric luminescent element in which an organic compound is electrically excited by recombination of injected electrons and holes to emdt light. Since the report of Tang et al. from Eastman Kodak Company indicating that an organic multilayer thin-film element emits light at a high luminance (Non-patert Literature 1), many companies and research institutes have conducted research on organic EL elements. In the typical configuration of the organic EL element of Eastman Kodak Company, a diamine compound as a hole transport material, tris(8-quinolinolato)aluminum (III) as a luminescent material, and Mg:AG as a cathode are sequentially stacked on an ITO (indium tin oxide) glass substrate as a transparent anode. With the configuration, green light emission of about 1000 cd/cm² has been observed at a driving voltage of around 10 V. Stacked organic EL elements now investigated and practically used basically follow this configuration of Eastman Kodak Company.

In a stacked organic EL element, the performance of an electron transport layer, an electron injection layer, and a hole transport layer to be provided between the organic electroluminescent layer and electrodes greatly influences the device characteristics. Thus, research and development for improving the performance of these layers has been intensively conducted, and a large number of improvements research on the electron transport layer and electron injection layer also have been reported.

For example, Patent Literature 1 suggests a configuration in which an electron injection layer has properties improved by co-depositing an electron-transporting organic compound and a metal compound containing an alkali metal, which has a low work function (low electronegativity), to mix. the metal. compound into the electron in layer. Patent Literature 2 suggests use of a phosphine oxide compound as an electron transporting material. Further, Patent Literature 3 suggests a method for doping an. organic compound having coordination sites with an alkali metal, for constituting an electron transport layer.

Meanwhile, methods for producing an organic EL element may be broadly classified into deposition processes, in which various materials are deposited on the substrate, and wet processes, in which solutions of various materials are applied onto the substrate and the solutions applied are dried. Such wet processes have advantages such as no requirement of vacuum, high productivity, easy formation. of a large area, and thus production of a stacked organic EL element by a wet process is considered. to be increasingly important in the future.

Methods of producing a stacked organic EL element by a wet process are broadly classified into two types: one is a method in which the lower layer is formed and then insolubilized by crosslinking or polymerizing the layer via heat or light, and thereafter the upper layer is formed; and the other is a method in which the material used in the upper layer has solubility markedly different from that of the material used in the lower layer. In the former method, while the material may be chosen from many options, removal of the initiator and unreacted material is difficult after the crosslinking or polymerization reaction, resulting in a problem in durability. In contrast, in the latter method, the choice of the materials is difficult, whereas it is possible to construct an element having higher purity and higher durability in comparison with the former method because no chemical reaction such as crosslinking or polymerization reaction is involved. As mentioned above, the latter method utilizing the difference in solubility between the constituent materials of each of the layers, despite of having difficulty in choice of materials, is more suitable for production of a stacked organic EL element by a wet process. However, one of the factors that make stacking by use of the difference in solubility between the materials each constituting each layer difficult is that most of the conducting polymers and spin-coatable organic semiconductors are soluble only in solvents having relatively high solubilizing ability such as toluene, chloroform, and tetrahydrofuran. Thus, formation of a hole transport layer or a light emitting layer followed by formation of a subsequent layer using a similar solvent leads to erosion in the underlying hole transport layer or light emitting layer, failing to form a stacked structure having a flat interface with few defects, which is problematic. Especially, in use of an ink-jet method, since the solvent is removed by natural drying, the residence time of the solvent becomes longer. For this reason, the hole transport layer or the light emitting layer is severely eroded, and thus it may be extremely difficult to achieve device characteristics which are of no problem in practical use.

However, the electron injection layers, the electron transporting materials, and the electron transport layers according to Patent Literatures 1 to 3 are all intended to lower the operation voltage and to improve the emission efficiency. Accordingly, it can be hardly said that formation of a multilayer structure by a wet process and improvement in the durability thereof have been achieved. Moreover, these inventions, in which the electron transport layer and the electron injection layer are formed by a vacuum deposition method, require large-scale equipment as well as have difficulty in precisely adjusting the deposition rates in simultaneous deposition of two or more materials, thereby also having a problem of having poor productivity.

Patent Literature 4 suggests a metal complex including, as a ligand, heteroaryl or a derivative thereof, the metal complex being useful for a charge transport material for EL elements. However, it cannot be said that formation of a multilayer structure by a wet process and improvement in the durability thereof have been sufficiently considered.

The present inventors thus have developed an alcohol-soluble material that may be applied onto a hole transport layer or a light emitting layer formed respectively of a hole transport material or an emissive material generally sparingly soluble in alcohol (Patent Literatures 5 and 6).

### CITATION LIST

### Patent Literature

Patent Literature 1: Japanese Patent Laid-Open No. 2005-63910
Patent Literature 2: Japanese Patent Laid-Open No. 2002-63989
Patent Literature 3: Japanese Patent Laid-Open No. 2002-352961
Patent Literature 4: U.S. Patent Application Publication No. 2018/0175307
Patent Literature 5: International Publication No. WO 2011/021385
Patent Literature 6: International Publication. No. WO 2018/021406

### Non Patent Literature

Non Patent Literature 1: “Organic electroluminescent diodes”, written by C. W. Tang and S. A. VanSlyke, Applied Physics Letters (U.S.), 21 Sep. 1987, Vol. 51, No. 12, 913-915, The American. Institute of Physics

### SUMMARY OF INVENTION

### Technical Problem

In production of an organic EL element by a wet process, the production is often started from the anode side, and a solvent for a liquid material to form the hole transport layer is relatively freely chosen. Meanwhile, a solvent for the liquid material to form the electron transport layer is limited by the solubility of the hole transport layer or the light emitting layer. Thus, at present, for the wet process, the electron transporting material is less freely chosen than for a deposition method. Novel materials having electron transportability that can be used also for wet processes can broaden options of electron transporting materials.

The electron transporting materials described in Patent Literatures 5 and 6 have had room for improvement with respect to durability. For this reason, there have been desired novel materials that are expected to have further improved performance.

The present invention has been made under the above-described circumstances, and it is an object of the present invention to provide an alkali metal complex and an alkaline earth metal complex (hereinafter, may be simply referred to as the “metal complex”) that have both electron transportability and alcohol solubility. It is another object to provide a coordinating compound constituting the alkali metal complex and the alkaline earth metal complex. It is still another object to provide an electron transporting material that can be formed by a wet process in production of an organic electroluminescent element in which the metal cow les used and that has a multilayer structure, the electron transporting material having excellent electron injection properties, electron transport properties, and durability. It is further still another object to provide an organic electroluminescent element in which the electron transporting material is used.

### Solution to Problem

A first aspect of the present invention meeting the objects relates to a novel metal complex as follows. A metal complex having a novel coordinating compound of the present invention is a novel metal complex having both electron transportability and alcohol solubility, suitable for an electron transporting material for organic electroluminescent elements. Use of the metal complex singly or as an electron transporting material containing a metal alkoxide causes the durability of an electroluminescent element to be improved.

<1> A metal complex represented by the following Formula (1) to the following Formula (3), comprising at least one phenanthrolinyl group and a nitrogen-containing fused ring:

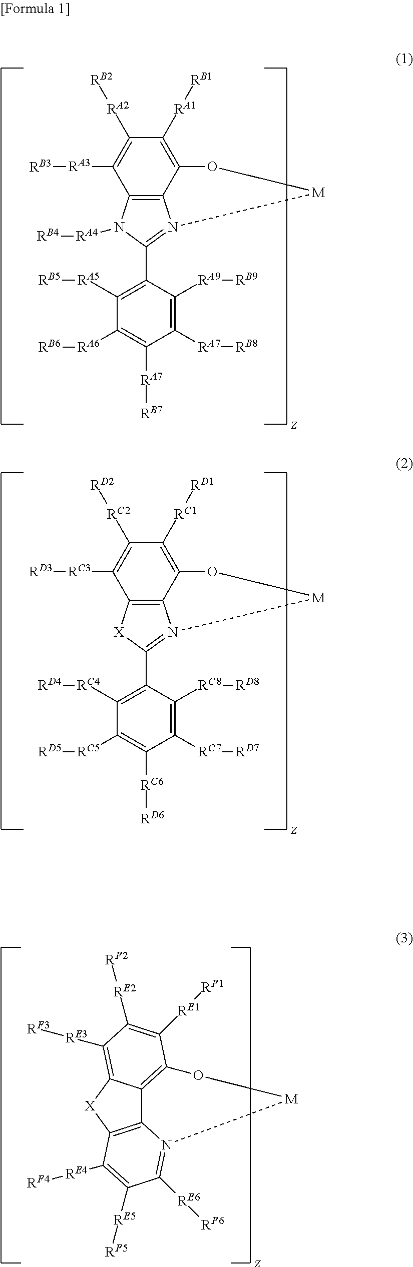

wherein, in Formula (1) to Formula (3),

R^{A1} to R^{A9}, R^{C1} to R^{C8}, and R^{E1} to R^{E6} are each independently a single bond, an alkylene group, an arylene group, a heteroarylene group, or a group represented by the following Formula (4):

### [Formula 2]

—R^{P1}—P(═O)R^{P2}—R^{P3}— (4)

wherein, in Formula (4), R^{P1} and R^{P3} are each independently a single bond, as alkylene group, as arylene group, or a heteroarylene group, and R^{P2} is an alkyl group, as aryl group, or a heteroaryl group,

R^{B1} to R^{B9}, R^{D1} to R^{D8}, and R^{F1} to R^{F6} are each independently a hydrogen atom, as alkyl group, an aryl group, a heteroaryl group, an alkoxy group, an aryloxy group, a heteroaryloxy group, an amino group, a cyano group, a halogen atom, or a hydroxy group,

one or more selected from the group consisting of R^{B1} to R^{B9} are a phenanthrolinyl group, one or more selected from the group consisting of R^{D1} to R^{D8} are a phenanthrolinyl group, and one or more selected from the group consisting of R^{F1} to R^{F6} are a phenanthrolinyl group,

M is as alkali metal or as alkaline earth metal,
Z is 1 or 2, and
X is O or S.

<2> The metal complex according to the <1>, wherein the phenanthrolinyl group is selected from the group consisting of the following Formula (5a) to the following Formula (5d):

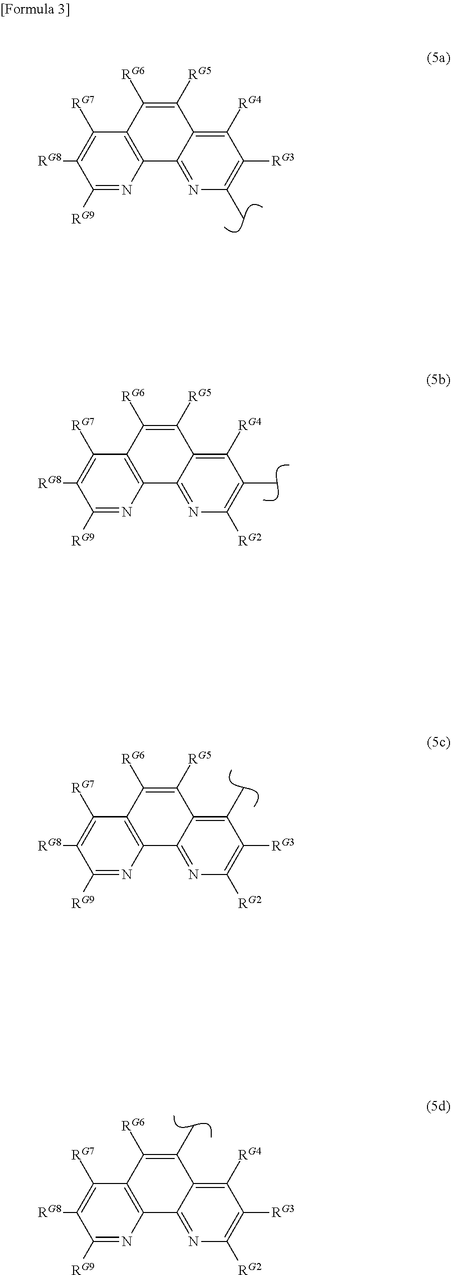

wherein, in Formula (5a) to Formnla (5d), R^{G2} to R^{G9} are each independently a hydrogen atom, an alkyl group, an aryl group, a heteroaryl group, an alkoxy group, an aryloxy group, a heteroaryloxy group, an amino group, a cyano group, a halogen atom, a hydroxy group, or a group represented by the following General Formula (6):

### [Formula 4]

—R^{P4}—P(═O)R^{P5}—R^{P6} (6)

wherein, in Formula (6), R^{P4} is a single bond, an alkylene group, an arylene group, or a heteroarylene group, and R^{P5} and R^{P6} are each independently an alkyl group, an aryl group, or a heteroaryl group.

<3> The metal complex according to the <1> or <2>, wherein the R^{A1} to R^{A9}, the R^{C1} to R^{C8}, and the R^{E1} to R^{E6} are each independently a single bond, an alkylene group having 1 to 4 carbon atoms, a phenylene group, a naphthylene group, a pyridylene group, a bipyridylene group, a pyrimidinylene group, or a group represented by the above Formula (4).

<4> The metal complex according to any of the <1> to <3>, wherein the R^{B1} to the R^{B9}, to R^{D1} to R^{D8}, and the R^{F1} tc R^{F6} are each independently a hydrogen atom or a phenanthrolinyl group.

<5> The metal complex according to any of the <1> to <4>, wherein the metal complex is any selected from the group consisting of compounds represented by the following L101-M to L108-M, L201-M to L-212-M, and L301-M to L320-M:

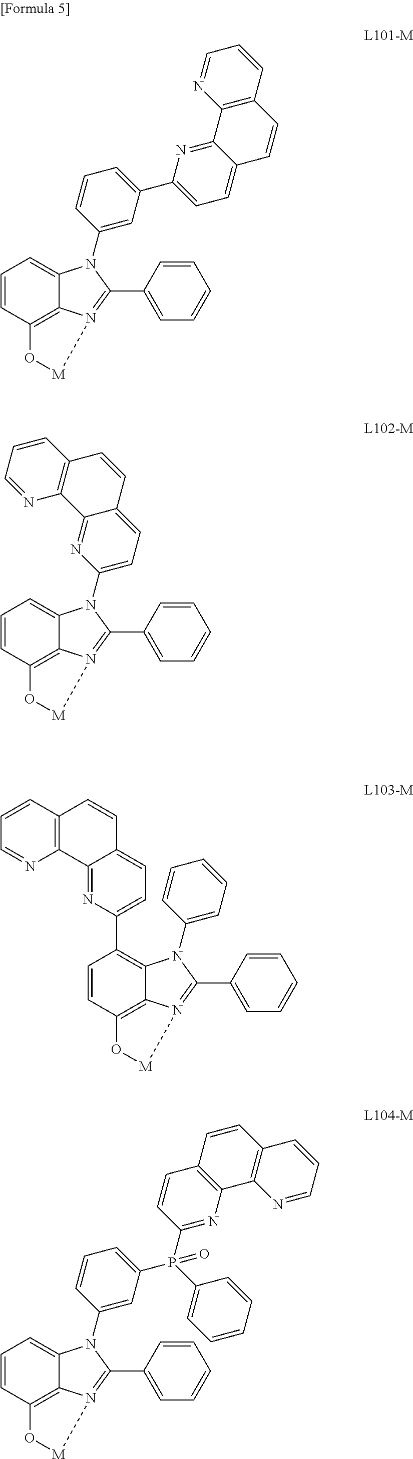

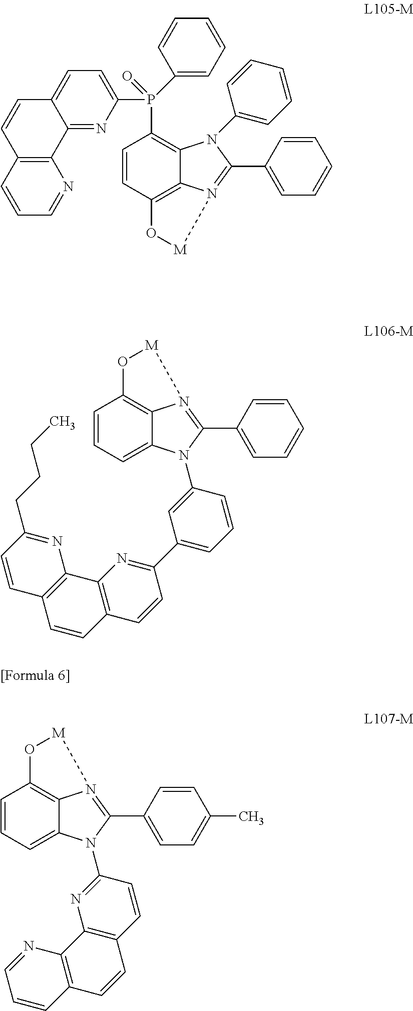

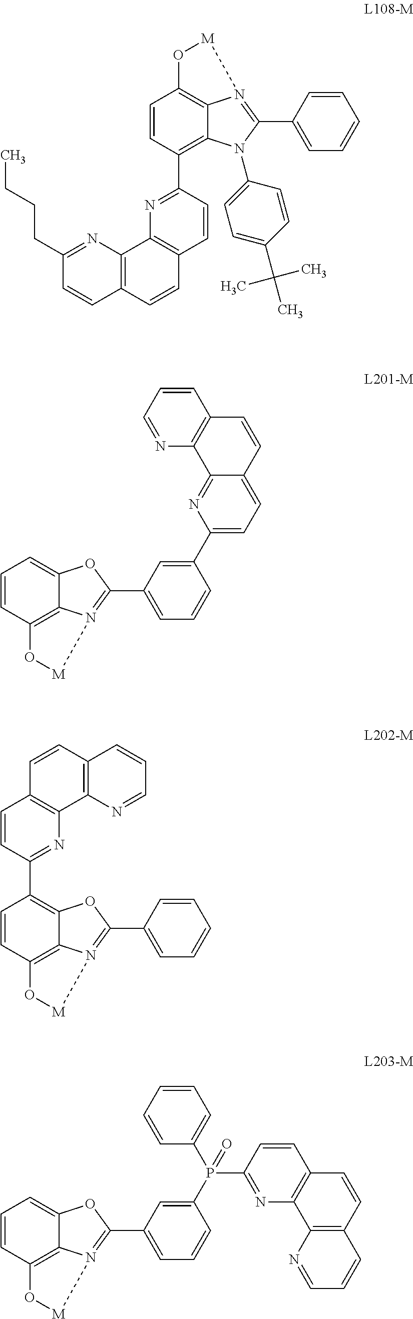

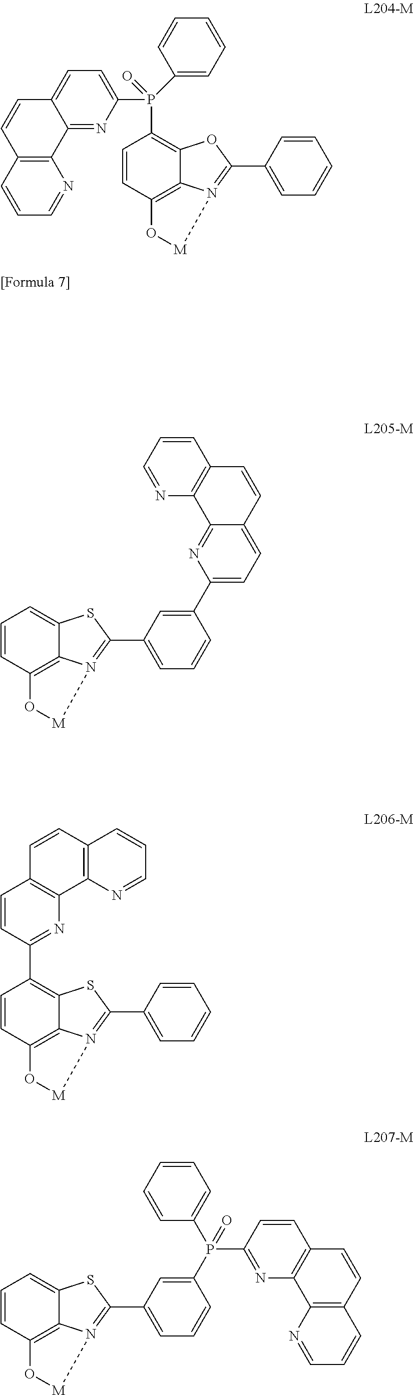

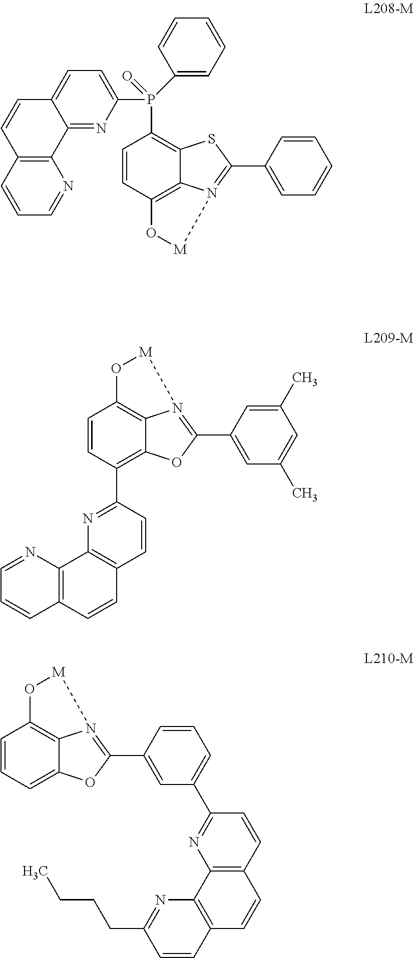

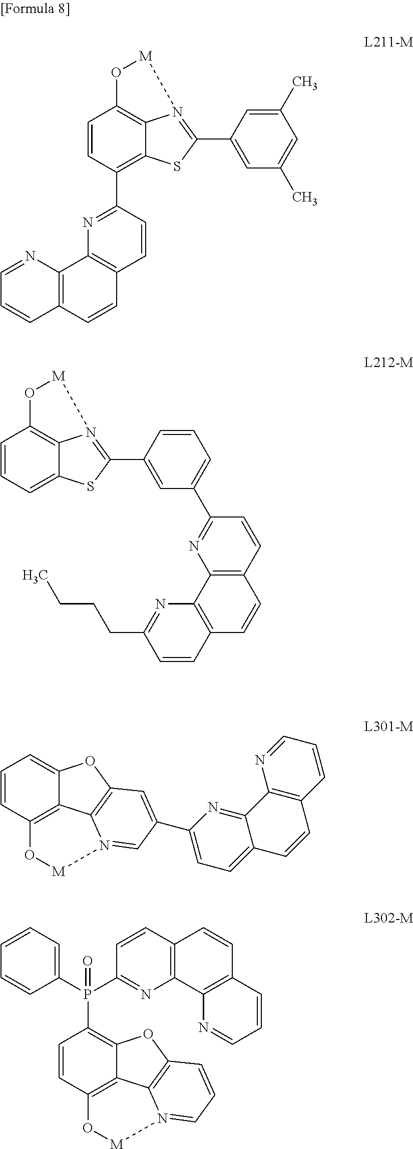

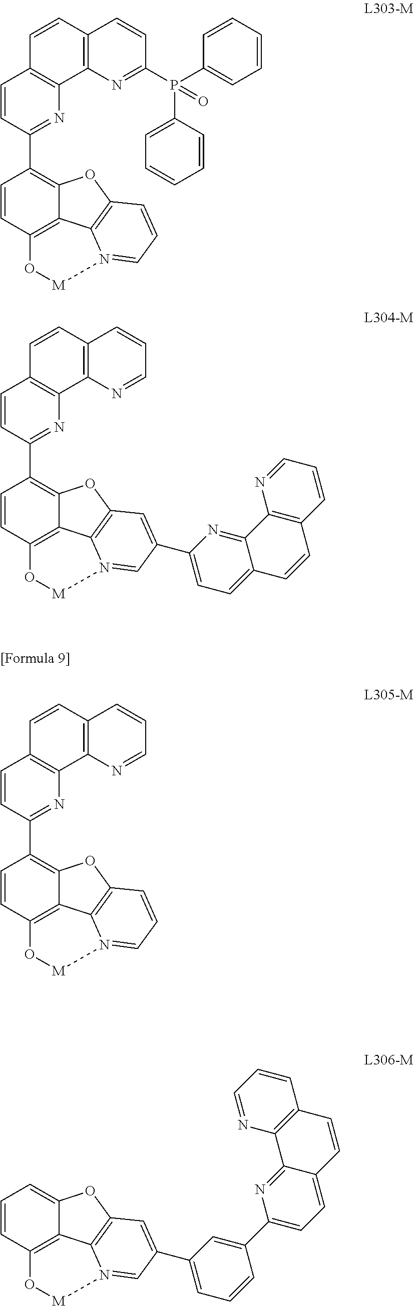

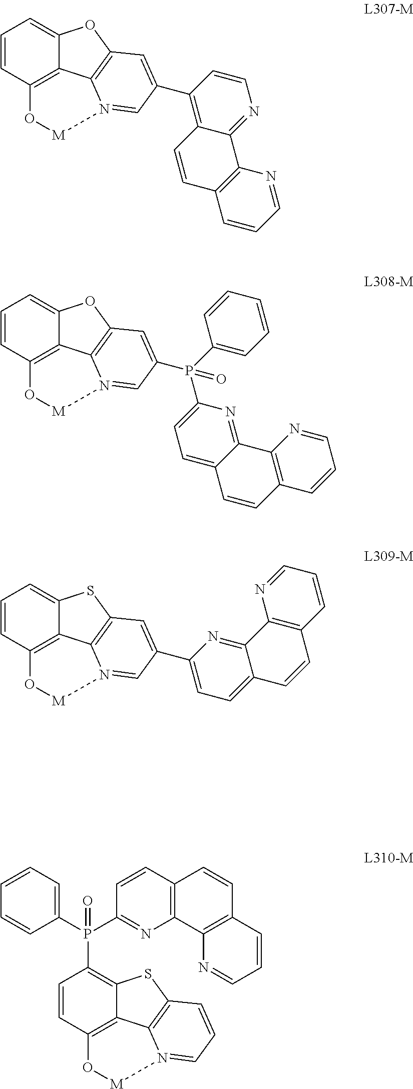

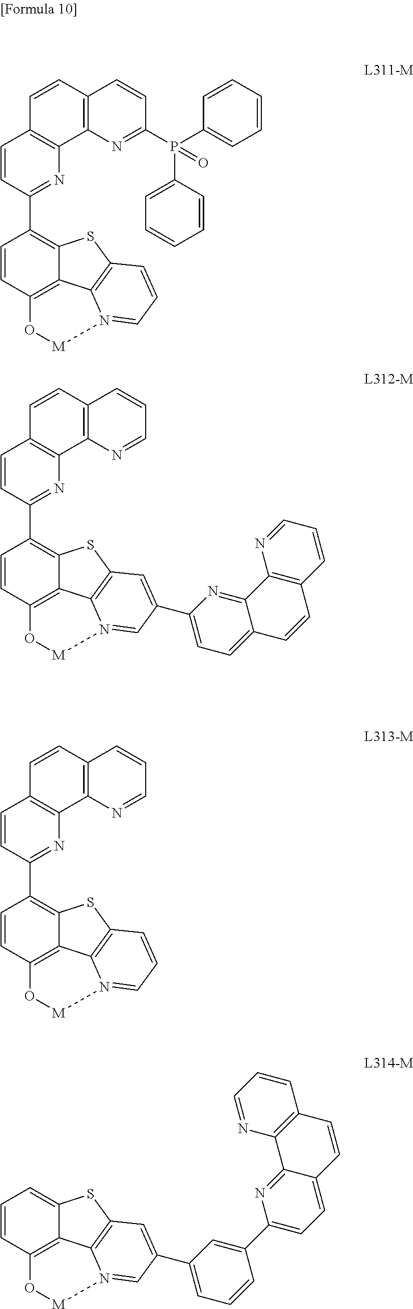

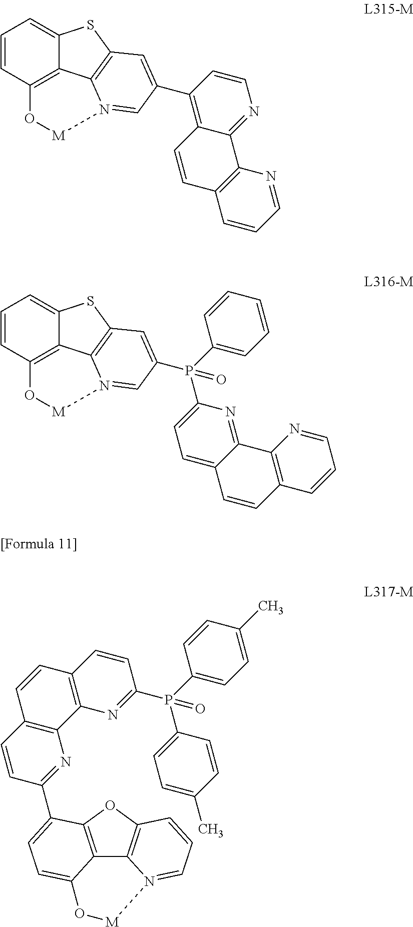

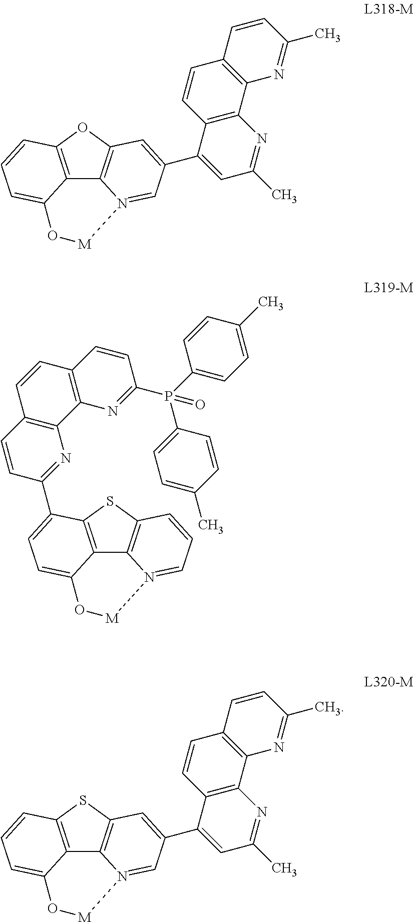

<6> The metal complex according to any of the <1> to <5>, wherein the M is an alkali metal.

<7> The metal complex according to the <6>, wherein. the alkali metal is Rh or Cs.

Next, a second aspect of the present invention meeting the objects relates to a coordinating compound for use in the above-described metal complex.

<8> A coordinating compound for use in the metal complex according to any of the <1> to <7>.

Next, a third aspect of the present invention meeting the objects relates to the following electron transporting material that can be formed by a wet process in production of an organic electroluminescent element in which the above-described metal complex is used and that has a multilayer structnre, the electron transporting material having excellent electron injection properties, electron transport properties, and durability.

<9> An electron transporting material for an organic electroluminescent element, comprising the metal complex according to any of the <1> to <7>.

<10> The electron transporting material according to the <9>, further comprising a dopant.

<11> The electron transporting material according to the <10>, wherein the dopant contains a metal alkoxide represented by the following Formula (7a) and/or the following Formula (7b):

### [Formula 12]

R^{H1}—O-M¹ (7a)

R^{H1}—O-M²—O—R^{H2} (7b)

wherein in Formula (7a) and Formula (7b), R^{H1} and R^{H2} each independently represent an alkyl group, M¹ is an alkali metal, and M² represents an alkaline earth metal.

<12> The electron transporting material according to the <10> or <11>, wherein the dopant contains one or more selected from the group consisting of an alkali metal complex of guinolinolate, an alkali metal complex of pyridylphenolate, an alkali metal complex of bipyridylphenoiate, and an alkali metal complex of. isoquinolinylphenolate.

<13> The electron transporting material according to any of the <10> to <12>, wherein the dopant contains one or more selected from the group consisting of an alkali metal hydroxide, an alkali metal halide, an alkali metal carbonate, an alkali metal hydrogen carbonate, an organic acid salt of an alkali metal having 1 to 9 carbon atoms, an alkaline earth metal hydroxide, an alkaline earth metal halide, an alkaline earth metal carbonate, an alkaline earth metal hydrogen carbonate, and an organic acid salt of an alkaline earth metal having 1 to 9 carbon atoms.

<14> The electron transporting material according to any of the <9> to <13>, further comprising a ligand constituting the metal complex.

Next, a fourth aspect of the present invention meeting the objects relates to a liquid material for constructing the electron transport layer of the following organic electroluminescent element, the liquid material comprising the above-described electron transporting material and a solvent.

<15> A liquid material for constructing an electron transport layer of an organic electroluminescent element, the liquid material comprising the electron transporting material according to any of the <9> to <14> and a protic polar solvent.

<16> The liquid material according to the <15>, wherein the protic polar solvent is an alcoholic solvent having 1 to 12 carbon atoms.

<17> The liquid material according to the <16>, wherein the alcoholic solvent having 1 to 12 carbon atoms is a monohydric or dihydric alcohol.

<18> The liquid material according to any of the <15> to <17>, comprising 0.01 to 10% by weight of the metal complex according to any of the <1> to <7>.

Further, the other aspects of the present invention meeting the objects relate to the following invention.

<19> An organic electroluminescent element comprising an electron transport layer containing the electron transporting material according to any of the <9> to <14>.

<20> A method for producing an organic electroluminescent element, comprising a step of constructing an electron transport layer of an organic electroluminescent element by a wet process using the liquid material according to any of the <15> to <18>.

### Advantageous Effect of Invention

According to the present invention, provided are a novel alkali metal complex and a novel alkaline earth metal complex that have both electron transportability and alcohol solubility. Also provided is a coordinating compound constituting an alkali metal complex and an alkaline earth metal complex. Also provded are an electron transporting material that can be formed by a wet process in production of an organic electroluminescent element in which the metal complex is used and that has a multilayer structure, the electron transporting material having excellent electron injection properties, electron transport properties, and durability, and an organic electroluminescent element in which the electron transporting material is used.

The electron transporting material including the metal complex of the present invention can achieve both high electron transportability and high electron injectability and can be suitably used as an electron transporting material for organic electroluminescent elements.

An organic electroluminescent element that can be produced with high productivity at low costs, has excellent emission efficiency, and has high durability is provided by application of the present invention.

### BRIEF DESCRIPTION OF DRAWINGS

FIG. 1 schematically illustrates a longitudinal section of an organic electroluminescent element.

FIG. 2 shows NMR charts of a metal complex (L101-Cs) of the present invention and a ligand thereof (L101).

FIG. 3 shows NMR charts of a metal complex (L301-Cs) of the present invention and a ligand thereof (L301).

FIG. 4 shows an NMR chart of a metal complex (L302-Cs) of the present invention and a ligand thereof (L302).

### DESCRIPTION OF EMBODIMENTS

Subsequently, embodiments for embodying the present invention will be described to facilitate understanding of the present invention. The description of the constitutive elements is an example (a representative example) of the embodiment of the present invention, and the present invention is not limited to the following conterts as long as the gist of the invention is not changed. An expression “to”, when employed herein, is employed as an expression including the numeric values before and after the “to”.

### [1] Metal Complex

A metal complex according to a first embodiment of the present invention (hereinafter, may be referred to as “the metal complex of the present invention”) is a metal complex represented by any of the following Formula (1) to the following Formula (3), comprising at least one phenanthrolinyl group and a nitrogen-containing fused ring.

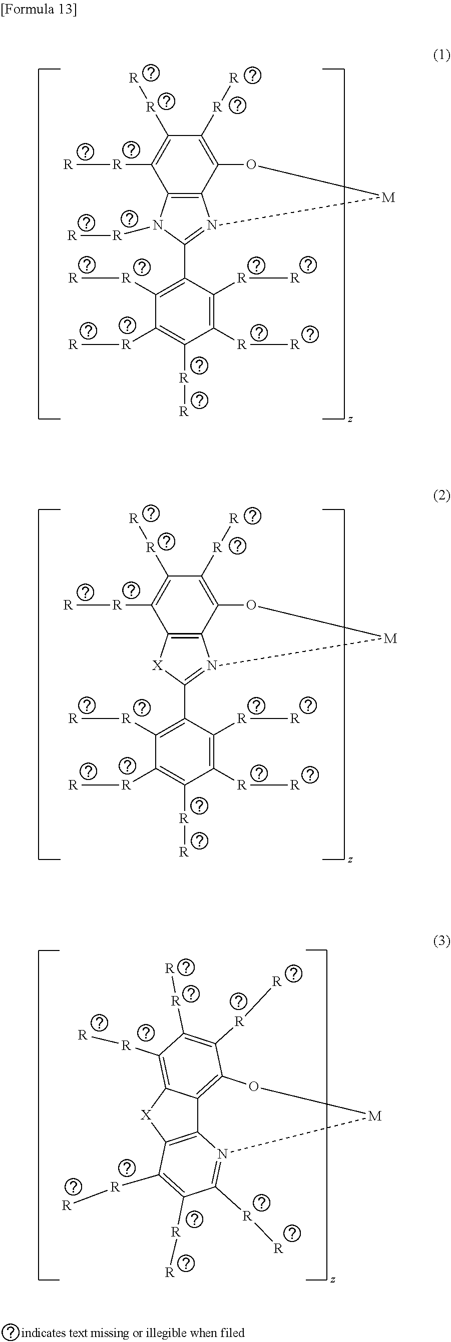

In Formula (1) to Formula (3),

R^{A1} to R^{A9}, R^{C1} to R^{C8}, and R^{E1} to R^{E6} are each independently a single bond, an alkylene group, an arylene group, a heteroarylene group, or a group represented by the following Formula (4):

### [Formula 14]

—R^{P1}—P(═O)R^{P2}—R^{P3}— (4)

wherein, in Formula (4), R^{P1} and R^{P3} are each independently a single bond, an alkylene group, an arylene group, or a heteroarylene group, and R^{P2} is an alkyl group, an aryl group, or a heteroaryl group,

R^{B1} to R^{B9}, R^{D1} to R^{D8}, and R^{F1} to R^{F6} are each independently a hydrogen atom, an alkyl group, an aryl group, a heteroaryl group, an alkoxy group, an aryloxy group, a heteroaryloxy group, an amino group, a cyano group, a halogen atom, or a hydroxy group,

one or more selected from the group consisting of R^{B1} to R^{B9} are a phenanthrolinyl group, one or more selected from the group consisting of R^{D1} to R^{D8} are a phenanthrolinyl group, and one or more selected from the group consisting of R^{F1} to R^{F6} are a phenanthrolinyl group,

M is an alkali metal or an alkaline earth metal,

Z is 1 or 2, and

X is O or S.

The metal complex of the present invention comprises at least one phenanthrolinyl group and a nitrogen-containing fused ring. In Formula (1), the basic skeleton is a benzimidazole complex. In Formula (2), X is O or S, the basic skeleton is a benzoxazole complex when X is O, and the basic skeleton is a benzothiazole complex when X is S. In Formula (3), X is O or S, the basic skeleton is a benzofuropyrdine complex when X is O, and the basic skeleton is a benzothienopyridine complex when X is S.

Each ligand constituting the metal complex of the present invention (coordinating compound) has a structure having a phenolate and a nitrogen-containing fused ring. formed by fusing two or more rings including a N-containing hetero ring, in which structure the nitrogen atom constituting the nitrogen-containing fused ring and the O-ion of the phenolate are coordinated to metal M. As mentioned above, it is presumed that allowing the portion of the ligand coordinated to the metal M to have a rigid skeleton has improved the stability of the coordination structure even in an anionic state and made the durability for an electron transporting material mentioned below excellent. It is also presumed that the phenanthrolinyl group contributes to improvement of the electron transportability and electron injectability, in addition to the improvement of the durability.

Here, in the present application, the nitrogen-containing fused ring is one formed by fusing two or more rings, at least one of the rings constituting the fused ring being a N-containing hetero ring that contains a nitrogen atom as a ring constituent element. In the present invention, the basic skeletons in the Formula (1) to the Formula (3) are configured to include a nitrogen-containing fused ring.

In the present application, a single bond represents a direct connection. For example, when R^{A1} is a single bond, a structure is represented in which R^{B1} is directly connected to the basic skeleton, not through any group of an alkylene group, an arylene group, a heteroarylene group, and the Formula (4). When R^{P1} in the Formula (4) is a single bond, a structure is represented in which the P atom is directly connected to the basic skeleton, not through any group of an alkylene group, an arylene group, and a heteroarylene group. When R^{P4} in Formula (6) mentioned below is a single bond, a structure is represented in which a P atom is directly connected to the phenanthroline skeleton, not through any group of an alkyle group, an arylene group, and a heteroarylene group.

In the present application, the alkylene group may be linear, branched, or cyclic. Examples of the alkylene group include a methylene group, an ethylene group, a n-propylene group, an iso-propylene group, a n-butylene group, a sec-butylene group, an iso-butylene group, and a tert-butylene group.

The alkylene group may be unsubstituted or may have a substituent. Examples of the substituent include an aryl group, a heteroaryl group, an alkoxy group, an aryloxy group, a heteroaryloxy group, and a halogen atom. Preferred are a phenyl group, a naphthyl group, a pyridyl group, a bipyridyl group, a phenanthrolinyl group, a fluorine atom, and the like. When the alkylene group has a. plurality of substituents, these may be the same or different.

In the present application, the arylene group may be monocyclic or polycyclic (a ring assembly in which two or more monocycles are linked or a fused ring in which two or more monocycles are fused). Examples of the arylene group include a phenylene group, a naphthylene group, an anthracenylene group, a pyrenylene group, and a biphenylene group (divalent biphenyl group).

The arylene group may be unsubstituted or may have a substituent. Examples of the substituent include an. alkyl group, an aryl group, a heteroaryl group, an alkoxy group, an aryloxy group, a heteroaryloxy group, and a halogen atom. Preferred are an alkyl group having 1 to 4 carbon atoms, a phenyl group, a naphthyl group, a pyridyl group, a bipyridyl group, a phenanthrolinyl group, and the like. When the arylene group has a plurality of substituents, these may be the same or different.

In the present application, the heteroarylene group may be monocyclic or polycyclic. Examples of the heteroarylene group include a pyridylene group, a pyrimidinylene group, a triazinylene group, a quinolylene group, an imidazolylene group, an oxarolylene group, a thiazolylene group, a carbonylilene group, a furylene group, a thienylene group, and a bipyridylene group (divalent bipyridyl group).

The heteroarylene group may be unsubstituted or may have a substituent. Examples of the substituent include an alkyl group, an aryl group, a heteroaryl group, an alkoxy group, an aryloxy group, a heteroaryloxy group, and a halogen atom. Preferred are an alkyl group having 1 to 4 carbon atoms, a phenyl group, a naphthyl group, a pyridyl group, a bipyridyl group, a phenanthrolinyl group, and the like. When the heteroarylene group has a plurality of substituents, these may be the same or different.

In the present application, the alkyl group may be linear, branched, or cyclic. Examples of the alkyl group include a methyl group, an ethyl group, a propyl group, a butyl group, a pentyl group, a hexyl group, a heptyl group, octyl, a nonyl group, a decyl group, and also structural isomers thereof.

The alkyl group may be unsubstituted or may have a substituent. Examples of the substituent include an aryl group, a heteroaryl group, an alkoxy group, an aryloxy group, a heteroaryloxy group, and a halogen atom. Preferred are a phenyl group, a. naphthyl group, a pyridyl group, a bipyridyl group, a phenanthrolinyl group, a fluorine atom, and the like. When the alkyl group has a plurality of substituents, these may be the same or different.

In the present application, the aryl group may be monocyclic or polycyclic. Examples of the aryl group include a phenyl group, a biphenyl group, a naphthyl group, an anthracenyl group, and a pyrenyl group.

In the present application, the aryl group may be unsubstituted or may have a substituent. Examples of the substituent include an alkyl group, an aryl group, a heteroaryl group, an alkoxy group, an aryloxv group, a heteroaryloxy group, and a halogen atom. Preferred are an alkyl group having 1 to 4 carbon atoms, a phenyl group, a naphthyl group, a pyridyl group, a bipyridyl group, a phenanthrolinyl group, and the like. When the aryl group has a plurality of substituents, these may be the same or different.

In the present application, the heteroaryl group may be monocyclic or polycyclic. At least one selected. as the substituent of the metal complex of the present invention is a phenanthrolinyl group (phenanthrolyl group), which is one of heteroaryl groups. The metal complex of the present invention may also have a heteroaryl group other than a phenanthrolinyl group, and examples of the heteroaryl group include a pyridyl group, a bipyridyi group, a pyrimidyl group, a triazinyl group, a quinolyl group, an imidazolyl group, an oxazolyl group, a thiazolyl group, a carbolinyl group, a furyl group, and thienyl group.

The heteroaryl group may be unsubstituted or may have a substituent. Examples of the substituent include an alkyl group, an aryl group, a heteroaryl group, an alkoxy group, an aryloxy group, a heteroaryloxy group, and a halogen atom. Preferred are an alkyl group having 1 to 4 carbon atoms, a phenyl group, a naphthyl group, a pyridyl group, a bipyridyl group, a phenanthrolinyl group, and the like. When the heteroaryl group has a plurality of substituents, these may be the same or different.

In the present application, an alkoxy group has a structure in which an alkyl group is bonded to an oxygen atom, and the alkyl group bonded to an oxygen atom may be linear, branched, or cyclic. Examples of the alkoxy group include a methoxy group, an ethoxy Group, a propoxy group, a butoxy group, a pentoxy group, a hexoxy group, a heptoxy group, an octoxy group, a nonanoxy group, a decanoxy group, and structural isomers thereof.

The alkoxy group may be unsubstituted or may have a substituent. Examples of the substituent include an aryl group, a heteroaryl group, an alkoxy group, an aryloxy group, a heteroaryloxy group, and a halogen atom. Preferred are a phenyl group, a naphthyl group, a pyridyl group, a bipyridyl group, a phenanthrolinyl group, and the like. When the alkoxy group has a plurality of substituents, these may be the same or different.

In the present application, an aryloxy group has a structure in which an aryl group is bonded to an oxygen atom, and the aryl group bonded to an oxygen atom may be monocyclic or polycyclic. Examples of the aryloxy group include a phenyloxy group (phenoxy group), a naphthyloxy group, an anthracenyloxy group, and a pyrenyloxy group.

The aryloxy group may be unsubstituted or may have a substituent. Examples of the substituent include an alkyl group, an aryl group, a heteroaryl group, an alkoxy group, an aryloxy group, a heteroaryloxv group, and a halogen atom. Preferred are an alkyl group having 1 to 4 carbon atoms, a phenyl group, a naphthyl group, a pyridyl group, a bipyridyl group, a phenanthrolinyl group, and the like. When the aryloxy group has a plurality of substituents, these may be the same or different.

In the present application, a heteroaryloxy group has a structure in which a heteroaryl group is bonded to an oxygen atom, and the heteroaryl group bonded to an oxygen atom may be monocyclic or polycyclic. Examples of the heteroaryloxy group include a pyridyloxy group, a pyrimidyloxy group, a triazyloxy group, a quinblyloxy group, an imidazolyloxy group, an oxarolyloxy group, a thiazolyoxy group, a phenanthroiinyloxy group, a carbolinyloxy group, a furyloxy group, and a thienyloxy group.

The heteroaryloxv group may be unsubstituted or may have a substituent. Examples of the substituent include an alkyl group, an aryl group, a heteroaryl group, an alkoxy group, an aryloxy group, a heteroaryloxy group, and a halogen atom. Preferred are an alkyl group having 1 to 4 carbon atoms, a phenyl group, a naphthyl group, a pyridyl group, a bipyridyl group, a phenanthrolinyl group, and the like. When the heteroaryloxy group has a plurality of substituents, these may be the same or different.

In the present application, an amino group may be unsubstituted or may have a substituent. Examples of the substituent include an alkyl group, an aryl group, a heteroaryl group, an alkoxy group, an aryloxy group, and a heteroaryloxy group, and preferred are phenyl and pyridyl. When the amino group has a plurality of substituents, these may be the same or different.

In the present application, examples of the halogen atom include a fluorine atom (F), chlorine atom (Cl), a bromine atom (Br), and an iodine atom (I).

Next, the metal complex represented by the above Formula (1) to Formula (3) will be described.

In the Formula (1) to Formula (3), R^{A1} to R^{A9}, R^{C1} to R^{C8}, and R^{E1} to R^{E6} are each independently a single bond, an alkylene group, an arylene group, a heteroarylene group, or a group represented by the above Formula (4) (i.e., “—R^{P1}—P(═O)P^{P2}—R^{P3}—”). Here, from the viewpoint of structural stability, solubility in a polar solvent, ease of synthesis, electron transportability and electron injectability for an electron transporting material, and the like, in R^{A1} to R^{A9}, R^{C1} to R^{C8}, and R^{E1} to R^{E6}, an alkylene group preferably has 1 to 4 carbon atoms, an arylene group preferably has 6 to 18 carbon atoms, and a heteroarylene group preferably has 3 to 17 carbon. atoms.

From the viewpoint of electron. transportability for an electron transporting material, ease of synthesis, and the like, R^{A1} to R^{A9}, R^{C1} to R^{C8}, and R^{E1} to R^{E6} are each independently preferably a single bond, an alkylene group having 1 to 4 carbon atoms, an arylene group having 6 to 18 carbon atoms, a heteroarylene group having 3 to 17 carbon atoms, or a group represented by the Formula (4), and more preferably a single bond, an alkylene group having 1 to 4 carbon atoms, a phenylene group, a naphthylene group, a pyridylene group, a bipyridylene group, a pyrimidinylene group, or a group represented by the Formula (4). These may have a substituent. For example, an alkyl group having 1 to 4 carbon atoms or an alkoxv group having 1 to 4 carbon. atoms may be further introduced for the purpose of improving the solubility.

R^{P1} and R^{P3} in the above Formula (4) (i.e., “—R^{P1}—P(═O)R^{P2}—R^{P3}—”) are each independently a single bond, an alkylene group, an arylene group, or a heteroarylene group. R^{P1} is a group to be bonded to the basic skeleton of the metal complex represented by the Formula (1) to Formula (3), and R^{P3} is a group to be bonded to R^{B1} or the like, R^{D1} or the like, or R^{F1} or the like. R^{P2} in the Formula (4) is an alkyl group, an aryl group, or a heteroaryl group. These groups represented by R^{P1}, R^{P2}, or R^{P3} may be unsubstituted or may have a substituent.

In R^{A1} to R^{A3}, R^{A5} to R^{A9}, R^{C1} to R^{C8}, and R^{E1} to R^{E6}, R^{P1} and R^{P3} in the Formula (4) are each independently preferably a single bond, an alkylene group having 1 to 4 carbon atoms, an arylene group having 6 to 18 carbon atoms, or a heteroarylene group having 3 to 17 carbon atoms. From the viewpoint of electron transportability for an electron transporting material, R^{P1} and R^{P3} each are preferably a single bond, an arylene group having 6 to 18 carbon atoms, or a heteroarylene group having 3 to 17 carbon atoms, more preferably a single bond or an arylene group having 6 to 18 carbon atoms, further preferably a single bond or a phenylene group.

In R^{A1} to R^{A3}, R^{A5} to R^{A9}, P^{C1} to R^{C8}, and R^{E1} to R^{E6}, R^{P2} in the Formula (4) is preferably an alkyl group having 1 to 4 carbon atoms, an aryl group having 6 to 18 carbon atoms, or a heteroaryl group having 3 to 17 carbon atoms. From the viewpoint of electron transportability for an electron transporting material, R^{P2} is preferably an aryl group having 6 to 18 carbon atoms or a heteroaryl group having 3 to 17 carbon atoms, more preferably an aryl group having 6 to 18 carbon atoms, further preferably a phenyl group.

For example, in R^{A1} to R^{A3}, R^{A5} to R^{A9}, R^{C1} to R^{C8}, and R^{E1} to R^{E6}, examples of the group represented by the Formula (4) include “—C₆H₄P(═O)C₆H₅—, wherein R^{P1} is a phenylene group, R^{P2} is a phenyl group, and R^{P3} is a single bond” or “—P(═O)C₆H₅—, wherein R^{P1} and R^{P3} each are a single bond, and R^{P2} is a phenyl group”.

In R^{A4}, R^{P1} in the Formula (4) is preferably an alkylene group having 1 to 4 carbon atoms, an arylene group having 6 to 18 carbon atoms, or a heteroarylene group having 3 to 17 carbon atoms. From the viewpoint of electron transportability for an electron transporting material, R^{P1} is preferably an arylene group having 6 to 18 carbon atoms or a heteroarylene group having 3 to 17 carbon atoms, more preferably an arylene group having 6 to 18 carbon atoms, further preferably a phenylene group.

In R^{A4}, R^{P2} in the Formula (4) is preferably an alkyl group having 1 to 4 carbon atoms, an aryl group having 6 to 18 carbon atoms, or a heteroaryl group having 3 to 17 carbon atoms. From the viewpoint of durability, R^{P2} is preferably an aryl group having 6 to 18 carbon atoms or a heteroaryl group having 3 to 17 carbon atoms, more preferably an aryl group having 6 to 18 carbon atoms, further preferably a phenyl group.

In R^{A4}, R^{P3} in the Formula (4) is preferably a single bond, an alkylene group having 1 to 4 carbon atoms, an arylene group having 6 to 18 carbon atoms, or a heteroarylene group having 3 to 17 carbon atoms. From the viewpoint of electron transportability for an electron transporting material, R^{P3} is preferably a single bond, an arylene group having 6 to 18 carbon atoms, or a heteroarylene group having 3 to 17 carbon atoms, more preferably a single bond or an arylene group having 6 to 18 carbon atoms, further preferably a single bond or a phenylene group.

For example, in R^{A4}, examples of the group represented by the Formula (4) include “—C₆H₄—P(═O)C₆H₅—, wherein R^{P1} is a phenylene group, R^{P2} is a phenyl group, and R^{P3} is a single bond”.

R^{B1} to R^{B9}, R^{D1} to R^{D8}, and R^{F1} to R^{F6} in the Formula (1) to the Formula (3) are each. independently a hydrogen atom, an alkyl group, an aryl group, a heteroaryl group, an alkoxy group, an aryloxy group, a heteroaryloxy group, an amino group, a cyano group, a halogen atom, or a hydroxy group. As mentioned below, at least one selected from the group consisting of R^{B1} to R^{B9} is a phenanthrolinyl group, at least one selected from the group consisting of R^{D1} to R^{D8} is a phenanthrolinyl group, and at least one selected from the group consisting of R^{F1} to R^{F6} is a phenanthrolinyl group. Here, from the viewpoint of structural stability, solubility in a polar solvent, ease of synthesis, and the like, in R^{B1} to R^{B9}, R^{D1} to R^{D8}, and R^{F1} to R^{F6}, an alkyl group and an alkoxy group each preferably have 1 to 4 carbon atoms, an aryl group and an aryloxy group each preferably have 6 to 18 carbon atoms, and a heteroaryl group and a heteroaryloxy group each preferably have 3 to 17 carbon atoms. These may have a substituent.

For example, R^{B1} to R^{B9}, R^{D1} to R^{D8}, and R^{F1} to R^{F6} can be each independently a hydrogen atom, an alkyl group having 1 to 4 carbon atoms, an aryl group having 6 to 18 carbon atoms, a heteroaryl group having 3 to 17 carbon atoms, or an alkoy group having 1 to 4 carbon atoms. Specifically, R^{B1} to R^{B9}, R^{D1} to R^{D8}, and R^{F1} to R^{F6} can be each independently a hydrogen atom, an alkyl group having 1 to 4 carbon atoms, a phenyl group, a biphenyl group, a naphthyl group, a pyridyl group, a bipyridyl group, a pyrimidyl group, a triazinyl group, a phenanthrolinyl group, a carbolinyl group, or an alkoxy group having 1 to 4 carbon atoms. Examples of the alkyl group having 1 to 4 carbon atoms include a methyl group, an ethyl group, a propyl group, an iso-propyl group, a n-butyl group, a sec-butyl group, an iso-butyl group, and a tert-butyl, and examples of the alkoxy group having 1 to 4 carbon atoms include alkoxy groups corresponding to alkyl groups having 1 to 4 carbon atoms.

From the viewpoint of electron transportability and durability for an electron transporting material, at least one selected from the group consisting of R^{B1} to R^{B9}, at least one selected from the group consisting of R^{D1} to R^{D8}, and at least one selected from the group consisting of R^{F1} to R^{F6} may be each independently an aryl group, a heteroaryl group, an aryloxy group, or a heteroaryloxy group. From the viewpoint of adjustment of the bandgap and. electron conduction level for an electron transporting material, emission efficiency, and heat resistance, at least one selected from the group consisting of R^{B1} to R^{B9}, at least one selected from the group consisting of R^{D1} to R^{D8}, and at least one selected from the group consisting of R^{F1} to R^{F6} may be each independently an alkyl group, an alkoxy group, an amino group, a cyano group, a halogen atom, or a hydroxyl group.

For example, R^{B1} to R^{B9}, R^{D1} to R^{F1} and R^{F1} to R^{F6} can be each independently a hydrogen atom, an aryl group having 6 to 18 carbon atoms, or a heteroaryl group having 3 to 17 carbon atoms. Specifically, R^{B1} to R^{B9}, R^{D1} to R^{D8}, and R^{F1} to R^{F6} can be each independently a hydrogen atom, a phenyl group, a biphenyl group, a naphthyl group, a pyridyl group, a bipyridyl group, a pyrimidyl group, a triazinyl group, a phenanthrolinyl group, or a carbolinyl group. Preferably, R^{B1} to R^{B9}, R^{D1} to R^{D8}, and R^{F1} to R^{F6} are each independently a hydrogen atom or a phenanthrolinyl group.

The metal complex of the present invention has at least one phenanthrolinyl group, as described above. In the Formula (1), at least one selected from the group consisting of R^{B1} to R^{B9} is a phenanthrolinyl group. In the Formula (2), at least one selected from the group consisting of R^{D1} to R^{D8} is a phenanthrolinyl group. In the Formula (3), at least one selected from the group consisting of R^{F1} to R^{F6} is a phenanthrolinyl group.

In the Formula (1), 1 to 4 selected from the group consisting of R^{B1} to each are preferably phenanthrolinyl group, 1 to 3 selected therefrom each are more preferably a phenanthrolinyl group, and 1 or 2 selected therefrom are further preferably a phenanthrolinyl group. Although the phenanthrolinyl group may further have a phenanthrolinyl group as a substituent, the number of phenanthrolinyl groups in a ligand is preferably 1 to 4, more preferably 1 to 3, further preferably 1 or 2. When the number of phenanthrolinyl groups is too large, the solubility in a solvent tends to decrease, the structure tends to be unstable to thereby lead to decrease in the durability for an electron transporting material, and synthesis tends to be difficult.

In the Formula (2), 1 to 3 selected from the group consisting of R^{D1} to R^{D8} each are preferably a phenanthrolinyl group, and 1 or 2 selected therefrom are more preferably a phenanthrolinyl group. Although the phenanthrolinyl group may further have a phenanthrolinyl group as a substituent, the number of phenanthrolinyl groups in a ligand is preferably 1 to 4, more preferably 1 to 3, further preferably 1 or 2. When the number of phenanthrolinyl groups is too large, the solubility in a solvent tends to decrease, the structure tends to be unstable to thereby lead to decrease in the durability for an electron. transporting material, and synthesis tends to be difficult.

In the Formula (3), 1 to 3 selected from the group consisting of R^{F1} to R^{F6} each are preferably a phenanthrolinyl group, and 1 or 2 selected therefrom are more preferably a phenanthrolinyl group. Although the phenanthrolinyl group may further have a phenanthrolinyl group as a substituent, the number of phenanthrolinyl groups in a ligand is preferably 1 to 4, more preferably 1 to 3, further preferably 1 or 2. When the number of phenanthrolinyl groups is too large, the solubility in a solvent tends to decrease, the structure tends to be less stable to thereby lead to decrease in the durability for an electron transporting material, and synthesis tends to be difficult.

Here, the phenanthrolinyl group is preferably selected from 1,10-phenanthrolinyl groups represented by the following Formula (5a) to the following Formula (5d). When the metal complex of the present invention has two or more phenanthrolinyl groups, the phenanthrolinyl groups may be the same or different and are preferably each independently one selected from groups represented by the following Formula (5a) to the following Formula (5d). Preferred among these is the following Formula (5a) or the following Formula (5c).

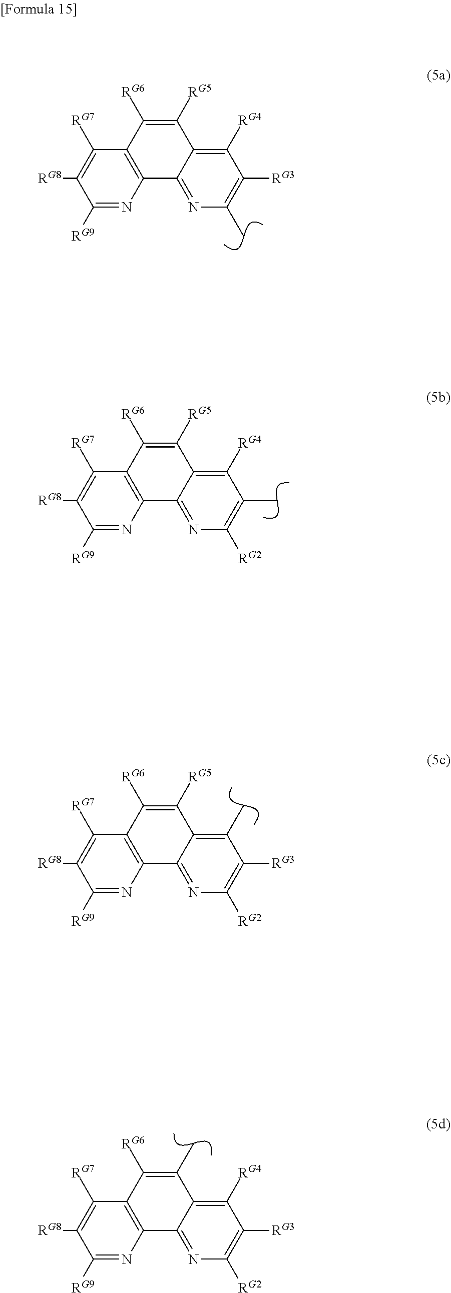

In the Formula (5a) to the Formula (5d), R^{G2} to R^{G9} are each independently a hydrogen atom, an alkyl group, an aryl group, a heteroaryl group, an alkoxy group, an aryloxy group, a heteroaryloxy group, an amino group, a cyano group, a halogen atom, a hydroxy group, or a group represented by the following Formula (6).

### [Formula 16]

—R^{P4}—P(═O)R^{P5}—R^{P6} (6)

In Formula (6), R^{P4} is a single bond, an alkylene group, an arylene group, or a heteroarylene group, and R^{P5} and R^{P6} are each independently an alkyl group, an aryl group, or a heteroaryl group. These groups represented by R^{P4}, R^{P5}, and R^{P6} may be unsubstituted or may have a substituent.

Here, in R^{G2} to R^{G9} in the Formula (5a) to the Formula (5d), from the viewpoint of structural stability, solubility in a polar solvent, ease of synthesis, and the like, an alkyl group and an alkoxy group each preferably have 1 to 4 carbon atoms, an aryl group and an aryloxy group each preferably have 6 to 18 carbon atoms, and a heteroaryl group and a heteroaryloxy group each preferably have 3 to 17 carbon atoms.

For example, R^{G2} to R^{G9} can be each independently a hydrogen atom, an alkyl group having. 1 to 4 carbon atoms, an aryl group having 6 to 18 carbon atoms, a heteroaryl group having 3 to 17 carbon atoms, an alkoxy group having 1 to 4 carbon atoms, or a group represented by the Formula (6). Specifically, R^{G2} to G^{G9} can be each independently a hydrogen atom, an alkyl group having 1 to 4 carbon atoms, a phenyl group, a biphenyl group, a naphthyl group, a pyridyl group, a bipyridyl group, a pyrimidyl group, a triazinyl group, a phenanthrolinyl group, a carbolinyl group, an alkoxy group having 1 to 4 carbon atoms, or a group represented by the Formula (6). These may have a substituent.

From the viewpoint of electron transportability and durability for an electron. transporting material, at least one selected from the group consisting of R^{G2} to R^{G9} may be an aryl group, a heteroaryl group, an aryloxy group, or a heteroaryloxy group. Introduction of a substituent, such as an alkyl group, an aryl group, a heteroaryl group, an alkoxy group, an aryloxy group, a heteroaryloxy group, an amino group, a cyano group, a halogen atom, a hydroxy group, or a group represented by the Formula (6), into R^{G9} in the Formula (5a) or R^{G2} and/or R^{G9} in the Formulas (5b) to (5d) also can improve durability for an electron transporting material. From the viewpoint of adjustment of the handgap and electron conduction level, emission efficiency, and heat resistance for an electron transporting material, at least one selected from the group consisting of R^{G2} to R^{G9} may be an alkyl group, an alkoxy group, an amino group, a cyano group, a halogen atom, a hydroxy group, or a group represented by the Formula (6).

For example, R^{G2} to R^{G9} can be each independently a hydrogen atom, an aryl group having 6 to 10 carbon atoms, a heteroaryl group having 3 to 17 carbon atoms, or a group represented by the Formula (6). R^{G2} to R^{G9} can be each independently a hydrogen atom, a phenyl group, a biphenyl group, a naphthyl group, a pyridyl group, a bipyridyl group, a pyrimidyl group, a triazinyl group, a phenanthrolinyl group, a carbolinyl group, or a group represented by the Formula (6). R^{G2} to R^{G9} also can be each independently a hydrogen atom or a group represented by the Formnla (6). R^{G2} to R^{G9} also can be each independently a hydrogen atom, an alkyl Group having 1 to 4 carbon atoms, an alkoxy group having 1 to 4 carbon atoms, or a group represented. by the Formula (6).

For the group represented. by the Formula (6), R^{P4} is preferably a single bond, an alkylene group having 1 to 4 carbon atoms, an arylene group having 6 to 18 carbon atoms, or a heteroarylene group having 3 to 17 carbon atoms. These may have a substituent. For example, R^{P4} can be a single bond, an alkylene group having 1 to 4 carbon atoms, a phenylene group, a naphthylene group, a pyridylene group, a bipyridylene group, or a pyrimidinylene group. R^{P4} also may be a single bond or a phenylene group.

R^{P5} and R^{P6} are preferably each independently an alkyl group having 1 to 4 carbon atoms, an aryl group having 6 to 18 carbon atoms, or a hetcroaryl group having 3 to 17 carbon atoms. For example, R^{P5} and R^{P6} are more preferably each independently an alkyl group having 1 to 4 carbon atoms, a phenyl group, a biphenyl group, a naphthyl group, a pyridyl group, a bipyridyl group, or a phenanthrolinyl group.

Specifically, examples of the group represented. by the Formula (6) include “—P(═O) (C₆H₅)₂, wherein R^{P4} is a single bond, and R^{P5} and P^{P6} each are a phenyl group.

From the viewpoint of structural stability, solubility in a polar solvent, ease of synthesis, and the like, in the phenanthrolinyl groups represented by the Formula (5a) to the Formula (5d), 2 to 7 selected from the group consisting of R^{G2} to R^{G9} each are preferably a hydrogen atom, 4 to 7 selected therefrom each are more preferably a hydrogen atom, and 6 or 7 selected therefrom each are more preferably a hydrogen atom. For example, R^{G3} and R^{G8} each can be a hydrogen atom. R^{G3}, R^{G4}, R^{G7}, and R^{G8} also each can be a hydrogen atom R^{G3} to R^{G8} also each can be a hydrogen atom.

In the metal complex represented by the Formula (1) to the Formula (3), M represents an alkali metal or alkaline earth metal. Examples of the alkali metal include Li, Na, K, Rb, and Cs, and examples of the alkaline earth metal include Be, Mg, Ca, Sr, and Ba.

As a metal complex for an electron transporting material mentioned below, alkali metals are more preferred. From the viewpoint of both electron injectability and solubility an alcohol, one having a larger atomic number is more preferred among these in the order of Li<Na<K<Rb<Cs, and Rb or Cs is more preferably used. As the alkaline earth. metal, Ba is more preferably used.

In the metal complex represented by. the Formula (1) to the Formula (3), Z represents an integer of 1 or 2. That is, when M is an alkali metal, Z is 1, and when M is an alkaline earth metal, Z is 2.

Next, the metal complex represented by the Formula (1) to the Formula (3) of the present invention will be described more in detail.

### (A) Metal Complex Represented by Formula (1)

Examples of the metal complex represented by Formula (1) of the present invention include complexes in which one or more selected from the group consisting of R^{B1}, R^{B3}, R^{B4}, and R^{B6} are a phenanthrolinyl group.

Examples thereof include complexes in which R^{A2}, R^{A5}, and R^{A7} to R^{A9} each are a single bond, R^{B2}, R^{B5}, and R^{B7} to R^{B9} each are a hydrogen atom, R^{A1}, R^{A3}, R^{A4}, and R^{A6} are each independently a single bond, an alkylene group, an arylene group, a heteroarylene group, or a group represented by the Formula (4), R^{B1}, R^{B3}, R^{B4}, and R^{B6} are each independently a hydrogen atom, an alkyl group, an aryl group, a heteroaryl group, an alkoxy group, an aryloxy group, a heteroaryloxy group, an amino group, a halogen atom, a cyano group, or a hydroxy group, and at least one of R^{B1}, R^{B3}, R^{B4}, and R^{B6} is a phenanthrolinyl group.

Examples thereof also include complexes in which R^{A1}, R^{A2}, and R^{A5} to R^{A9} each are a single bond, R^{B1}, R^{B2}, and R^{B5} to R^{B9} each are a hydrogen atom, R^{A3} and R^{A4} are each independently a single bond, an alkylene group, an arylene group, a heteroarylene group, or a group represented by the Formula (4),R^{B3} and R^{B4} are each independently a hydrogen atom, an alkyl group, an aryl group, a heteroaryl group, an alkoxy group, an aryloxy group, a heteroaryloxy group, an amino group, a halogen atom, a cyano group, or a hydroxy group, and at least one of R^{B3} and R^{B4} is a phenanthrolinyl group.

Additionally, examples of the metal complex represented by Formula (1) of the present invention include complexes in which one or more selected from the group consisting of R^{B3}, R^{B4}, and R^{B7} are a phenanthrolinyl group.

Examples thereof include complexes in which R^{A1}, R^{A2}, R^{A5}, R^{A6}, R^{A8}, and R^{A9} each are a single bond, R^{B1}, R^{B2}, R^{B5}, R^{B6}, R^{B9}, and R^{B9} each are a hydrogen atom, R^{A3}, R^{A4}, and R^{A7} are each independently a single bond, an arylene group having 6 to 18 carbon atoms, a heteroarylene group having 3 to 17 carbon atoms, or a group represented by General Formula (4), R^{B3}, R^{B4}, and R^{B7} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and at least one of R^{B3}, R^{B4}, and R^{B7} is a phenanthrolinyl group.

The complex may be one in which R^{A1} to R^{A9} each are a single bond, an arylene group having 6 to 18 carbon atoms, a heteroarylene group having 3 to 17 carbon atoms, or a group represented by General Formula (4), R^{B1} to R^{B3} and R^{B4} to R^{B9} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and R^{B4} is a phenanthrolinyl group.

The complex may be one in which R^{A1} to R^{A9} each are a single bond, as arylene group having 6 to 18 carbon atoms, a heteroarylene group having 3 to 17 carbon atoms, or a group represented by General Formula (4), R^{B1} to R^{B2} and R^{B4} to R^{B9} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and R^{B3} is a phenanthrolinyl group.

Specific examples are compounds represented by the following L101-M to L108-M. M represents an alkali metal or an alkaline earth metal. The following compounds are merely exemplary, and the metal complex of the present invention is not limited thereto.

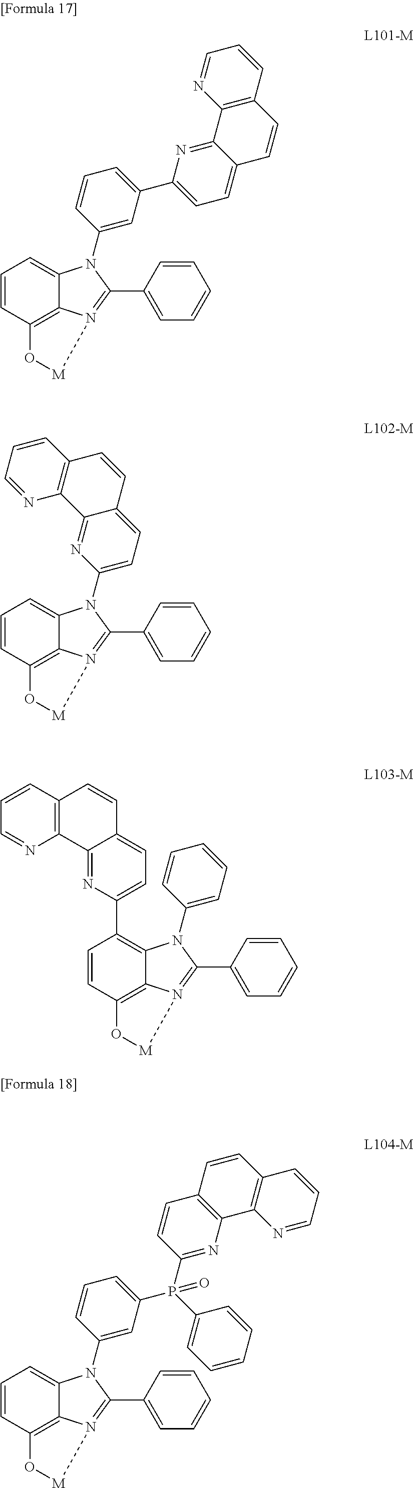

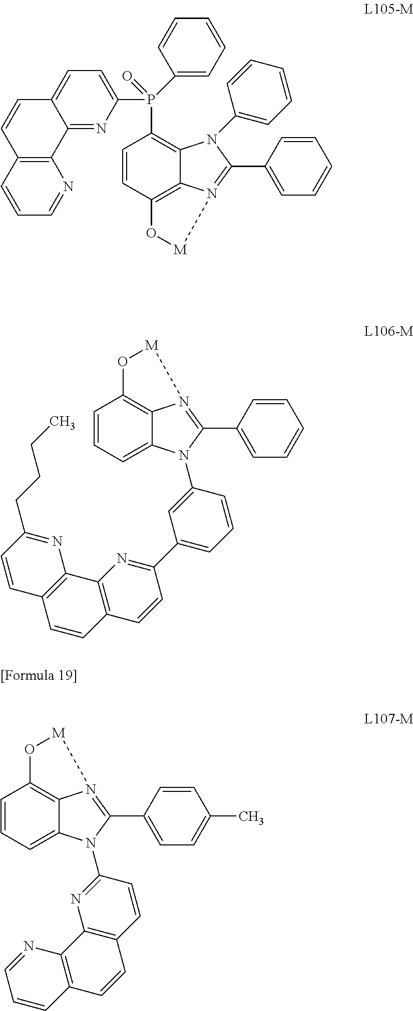

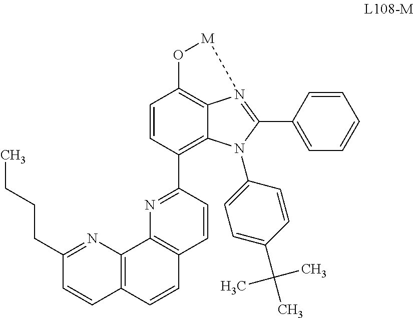

### (B) Metal Complex Represented by Formula (2)

Examples of the metal complex represented. by Formula (2) of the present invention include complexes in which one or more selected from the group consisting of R^{D1}, R^{D3}, and R^{D5} are a phenanthrolinyl group.

Examples thereof include complexes in which R^{C2}, R^{C4}, and R^{C6} to R^{C8} each are a single bond, R^{D2}, R^{D4}, and R^{D6} to R^{D8} each are a hydrogen atom, R^{C1}, R^{C3}, and R^{C5} are each independently a single bond, as alkylene group, as arylene group, a heteroarylene group, or a group represented by the Formula (4), R^{D1}, R^{D3}, and R^{D5} are each independently a hydrogen atom, an alkyl group, an aryl group, a heteroaryl group, an alkoxy group, an aryloxy group, a heteroaryloxy group, as amino group, a halogen atom, a cyano group, or a hydroxy group, and at least one of R^{D1}, R^{D3}, and R^{D5} is a phenanthrolinyl group.

Examples thereof also include complexes in which R^{C1}, R^{C2}, R^{C4}, and R^{C6} to R^{C8} each are a single bond, R^{D1}, R^{D2}, R^{D4}, and R^{D6} to R^{D8} each are a hydrogen atom, R^{C3} and R^{C5} are each independently a single bond, an alkylene group, an arylene group, a heteroarylene group, or a group represented by the Formula (4), R^{D3} and R^{D5} are each independently a hydrogen atom, an alkyl group, an aryl group, a heteroaryl group, an alkoxy group, an aryloxy group, a heteroaryloxy group, an amino group, a halogen atom, a cyano group, or a hydroxy group, and at least either one of R^{D3} and R^{D5} is a phenanthrolinyl group.

Additionally, examples of the metal complex represented by Formula (2) of the present invention include complexes in which one or more selected from the group consisting of R^{D3}, R^{D5}, and R^{D7} are a phenanthrolinyl group.

Examples thereof include complexes in which R^{C1}, R^{C2}, R^{C4}, R^{C6}, and R^{C8} each are a single bond, R^{D1}, R^{D2}, R^{D4}, R^{D6}, and R^{D8} each are a hydrogen atom, R^{C3}, R^{C5}, and RC7 are each independently a single bond, an arylene group having 6 to 18 carbon atoms, a heteroarylene group having 3 to 17 carbon atoms, or a group represented by General Formula (4), R^{D3}, R^{D5}, and R^{D7} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and at least one of R^{D3}, R^{D5}, and R^{D7} is a phenanthrolinyl group.

The complex also may be one in which R^{C1} to R^{C8} each are a single bond, an arylene group having 6 to 18 carbon atoms, a heteroarylene group having 3 to 17 carbon atoms, or a group represented by General Formula (4), R^{D1} to R^{D2} and R^{D4} to R^{B8} are each independently a hydrogen atom, an alkyl group having. 1 to 4 carbon atoms, an aryl group having 6 to 18 carbon atoms, a heteroaryl group having 3 to 17 carbon atoms, or an alkoxy group having 1 to 4 carbon atoms, and R^{D3} is a phenanthrolinyl group.

The complex also may be one in which R^{C1} to R^{C8} each are a single bond, an arylene group having 6 to 18 carbon atoms, a heteroarylene group having 3 to 17 carbon atoms, or a group represented by General Formula (4), R^{D1} to R^{D4} and R^{D6} to R^{D8} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and R^{D5} is a phenanthrolinyl group.

Specific examples are compounds represented by the following L201-M to L212-M. M represents an alkali metal or an alkaline earth metal. The following compounds are merely exemplary, and the metal complex of the present invention is not limited thereto.

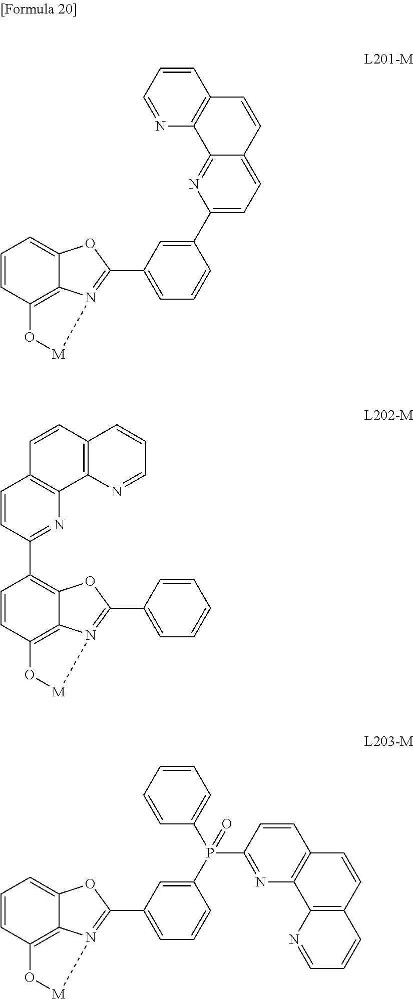

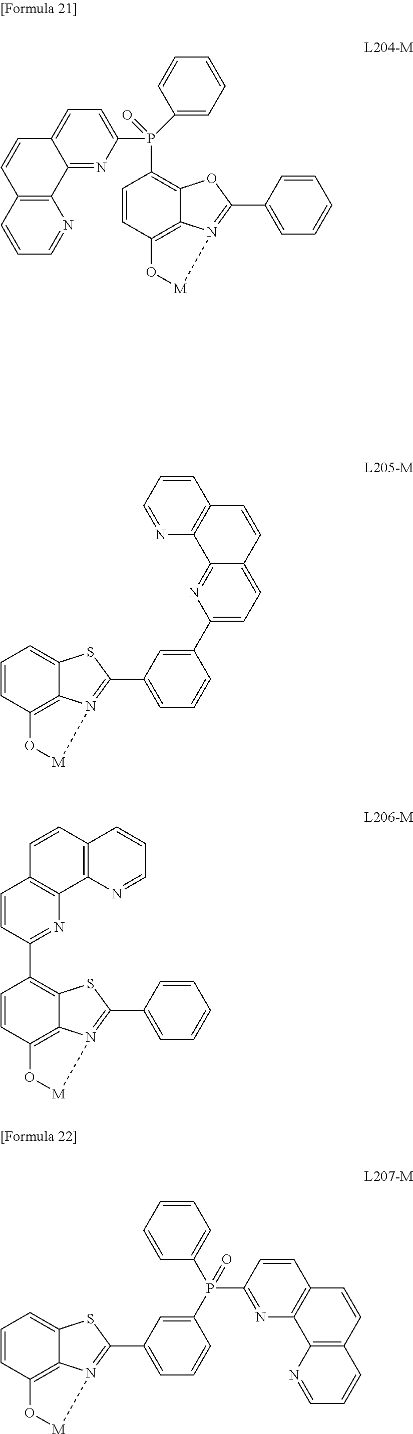

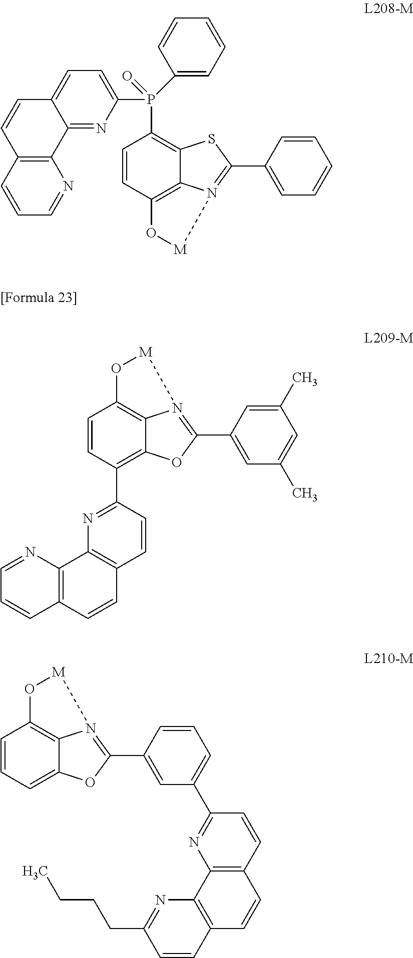

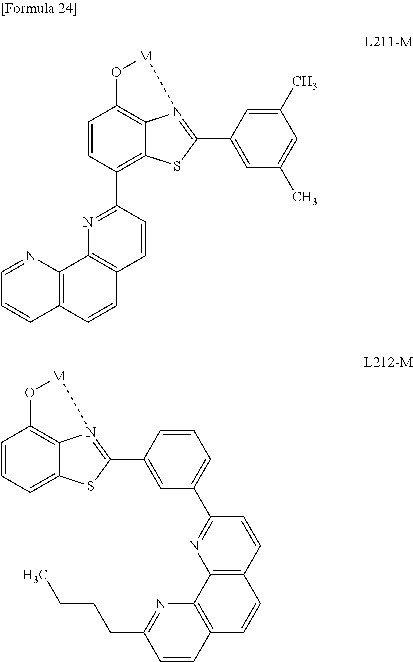

### (C) Metal Complex Represented by Formula (3)

Examples of the metal complex represented. by Formula (3) of the present invention include complexes is which one or more selected from the group consisting of R^{F1}, R^{F3}, and R^{F5} are a phenanthrolinyl group.

Examples thereof include complexes in which R^{E2}, R^{E4}, and R^{E6} are a single bond, R^{F2}, R^{F4}, and R^{F6} are a hydrogen atom, R^{E1}, R^{E3}, and R^{E5} are each independently a single bond, an alkylene group, an arylene group, a heteroarylene group, or a group represented by the Formula (4), R^{F1}, R^{F3}, and R^{F5} are each independently a hydrogen atom, an alkyl group, an aryl group, a heteroaryl group, an alkozy group, an aryloxy group, a heteroaryloxy group, an amino group, a halogen atom, a cyano group, or a hydroxy group, and at least one of R^{F1}, R^{F3}, and R^{F5} is a phenanthrolinyl group.

Examples thereof also include complexes in which R^{E1}, R^{E2}, R^{E4}, and R^{E6} each are a single bond, R^{F1}, R^{F2}, R^{F4}, and R^{F6} each are a hydrogen atom, R^{E3} and R^{E5} are each independently a sngle bond, an alkylene group, an arylene group, a heteroarylene group, or a group represented by the Formula (4), R^{F3} and R^{F5} are each independently a hydrogen atom, an alkyl group, an aryl group, a heteroaryl group, an alkoxy group, an aryloxy group, a heteroaryloxy group, an amino group, a halogen atom, a cyano group, or a hydroxy group, and at least either one of R^{F3} and R^{F5} is a phenanthrolinyl group.

The complex also may be one in which R^{E1} to R^{E6} each are a single bond, an arylene group having 6 to 18 carbon atoms, a heteroarylene group having 3 to 17 carbon atoms, or a group represented by General Formula (4), R^{F1} to R^{F2} and R^{F4} to R^{F6} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and R^{F3} is a phenanthrolinyl group.

Examples thereof also include complexes in which R^{E1} to R^{E6} each are a single bond, an arylene group having 6 to 18 carbon atoms, a heteroarylene group having 3 to 17 carbon atoms, or a group represented by General Formula (4), R^{F1} to R^{F4}, and R^{F6} are each independently a hydrogen atom, an alkyl group having 1 to 4 carbon atoms, an aryl group having 6 to 18 carbon atoms, a heteroaryl group having 3 to 17 carbon atoms, or an alkoxy group having 1 to 4 carbon atoms, and R^{F5} is a phenanthrolinyl group.

Specific examples are compounds represented by the following L301-M to L320-M. M represents an alkali metal or an alkaline earth metal. The following compounds are merely exemplary, and the metal complex of the present invention is not limited thereto.

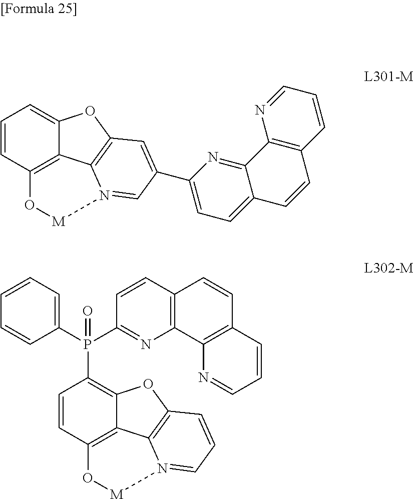

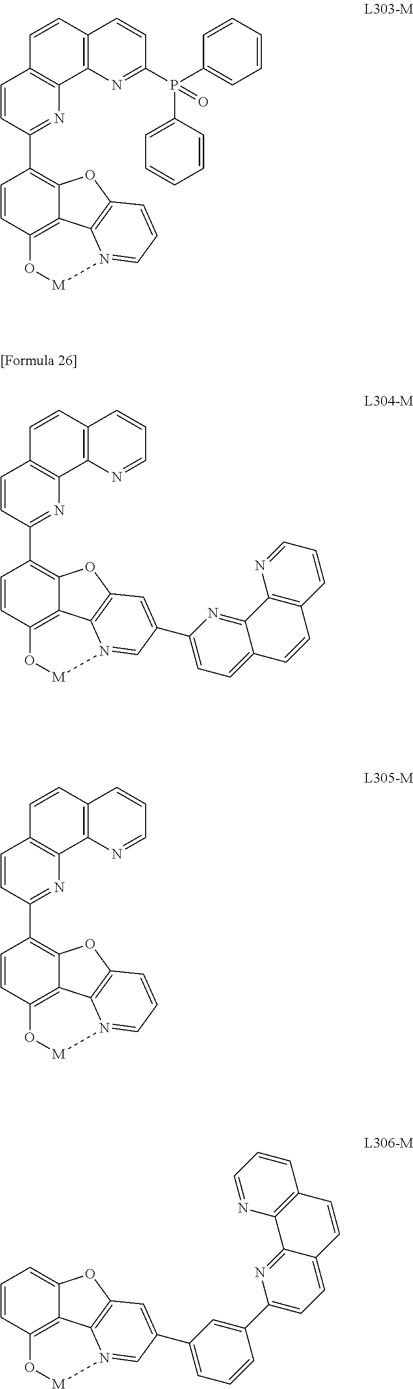

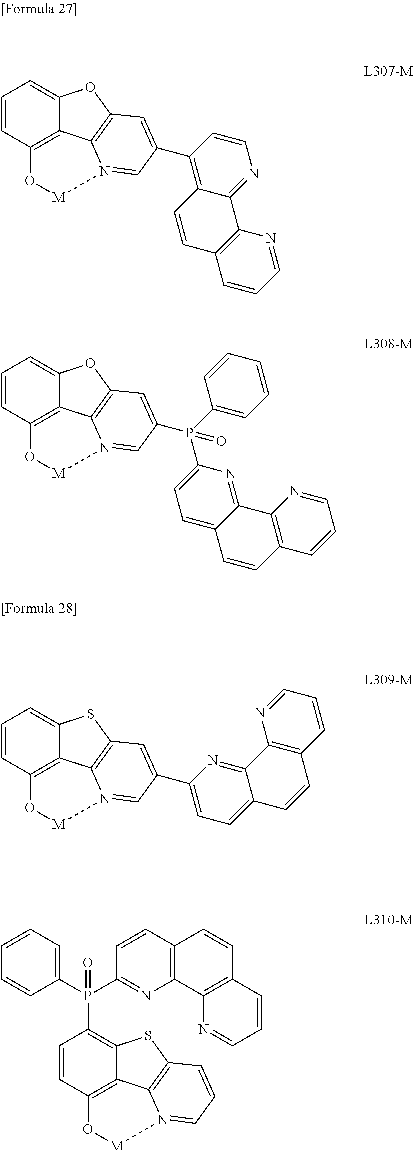

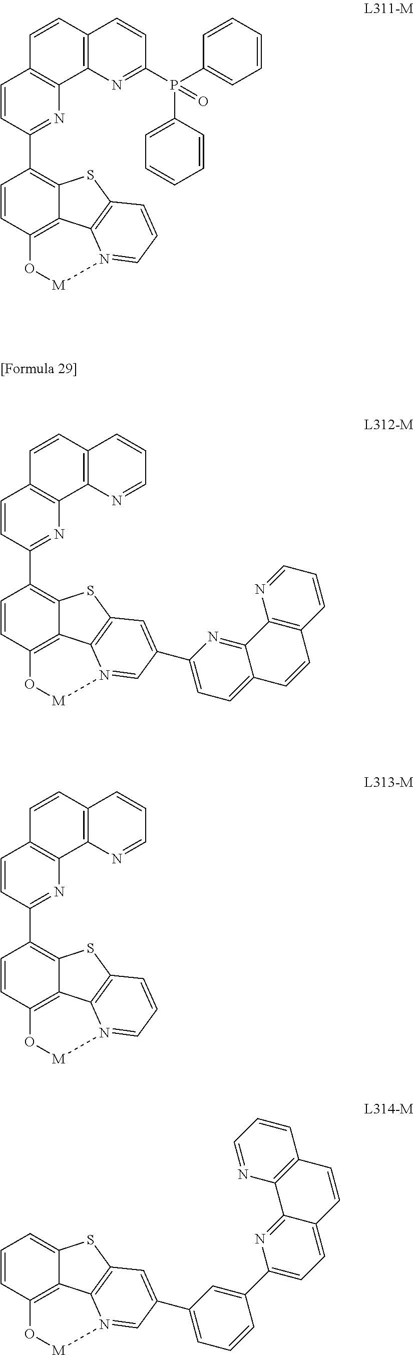

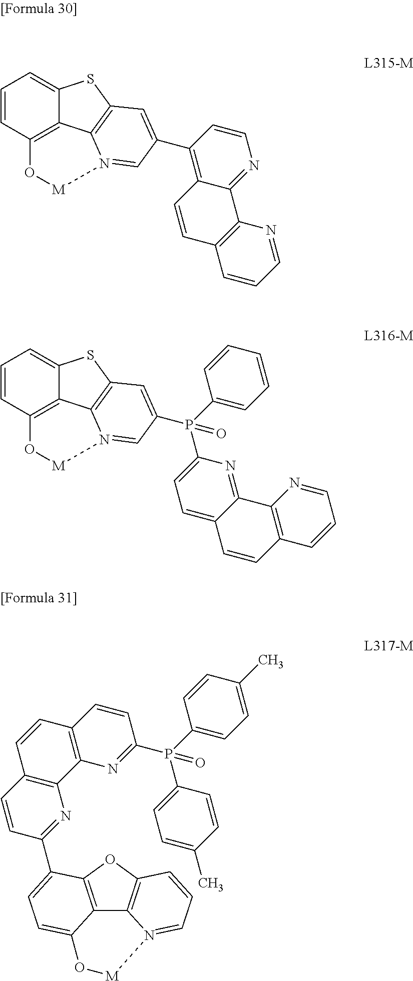

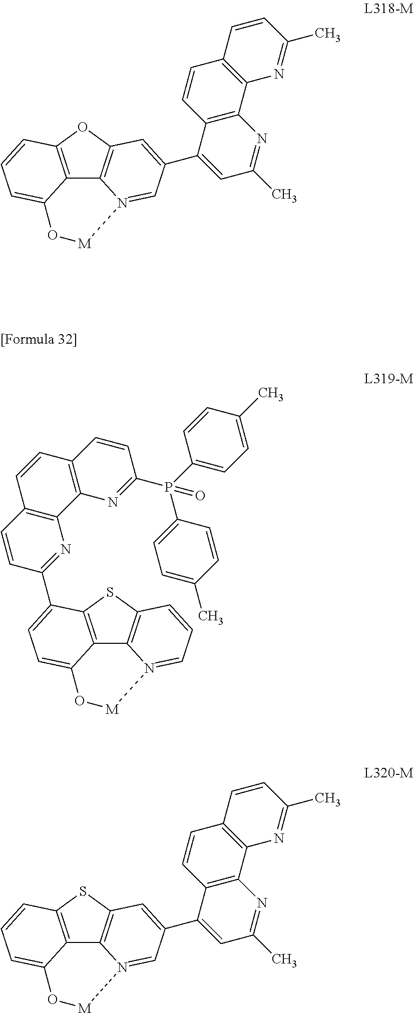

### (Production Method)

The metal complexes having a structure represented by the General Formulas (1) to (3) of the present invention can be synthesized by reacting a compound represented by the following Formula (1a) to the following Formula (3a) (ligand) with an alkali metal compound or alkaline earth metal compound as a metal ion source, for example.

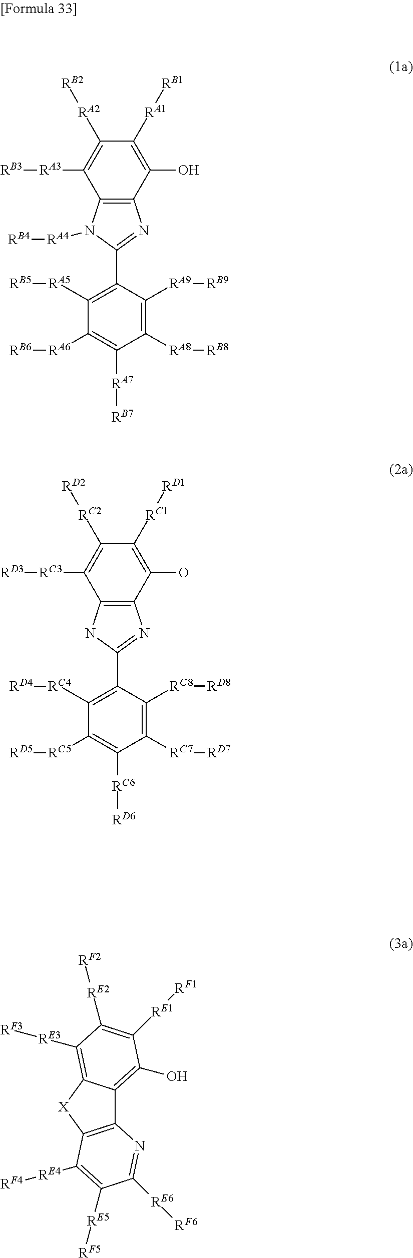

In the Formula (1a) to the Formula (3a), R^{A1} to R^{A9}, R^{C1} to R^{C8}, R^{E1} to R^{E6}, R^{B1} to R^{B9}, R^{D1} to R^{D8}, R^{F1} to R^{F6}, and X have the same meaning as in the Formula (1) to the Formula (3), and the same applies to preferred aspects.

When an alkali metal hydroxide or alkaline earth metal hydroxide is employed for example as the metal ion source, the metal complexes of the present invention will be given by the reaction of the following schemes.

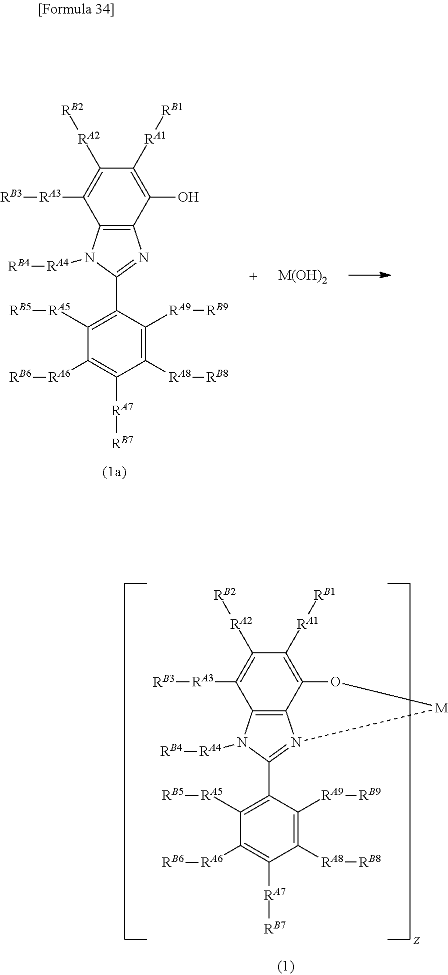

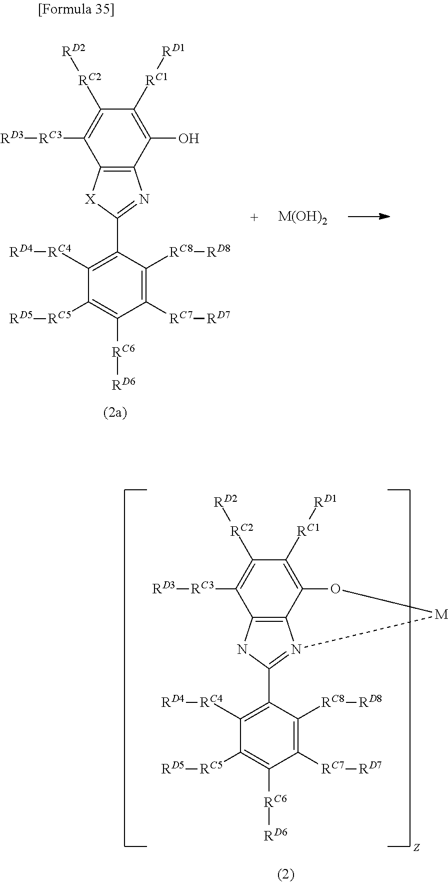

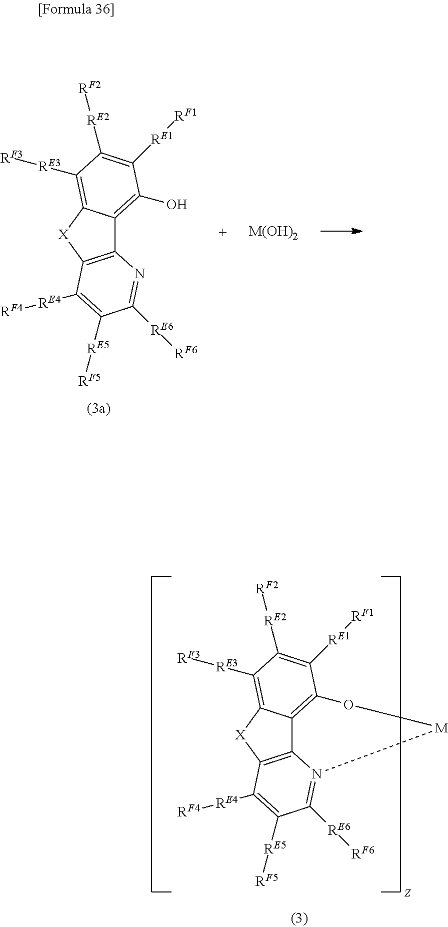

The molar ratio between the ligand and the metal ion source is adjusted as appropriate in accordance with the type of alkali metal compound or alkaline earth metal compound to be used and the valence number of the central metal of a metal complex to be synthesized. On reacting a ligand with an alkali metal compound, the reaction can be carried out so as to achieve a molar ratio of ligand: alkali metal ion in alkali metal compound=1:0.5 to 1:2 or 1:0.5 to 1:1.5. On reacting a ligand with an alkaline earth metal compound, the reaction can be carried out so as to achieve a molar ratio of ligand: alkaline earth metal ion in alkaline earth metal compound=1:0.25 to 1:1 or 1:0.25 to 1:0.8.

The reaction temperature and reaction time are only required to be adjusted as appropriate in accordance with the structure of a metal complex to be synthesized, type of metal ion source, and the like. The metal complex of the present invention can be synthesized by, for example, reacting a ligand with a metal ion source in the presence of a solvent at 20 to 30° C. for 0.5 to 25 hours. After the reaction of the ligand and the metal ion source, purjfication may be carried. out as appropriate. When a ligand and a metal ion source have sufficiently high purity, a solid to be given by removing the solvent may be used as is, without purification after the reaction, in an application such as an electron transporting material mentioned below. Although the solid to be given by removing the solvent may include an unreacted ligand or an unreacted metal ion source in addition to the metal complex of the present invention, depending on the molar ratio between the ligand and the metal ion source, such an unreacted ligand and. an unreacted metal ion can contribute to improvement in electron transportability of the electron transporting material and the like. Thus, the solid may be used with those included therein. The solid may also include a metal ion coordination-bonded to a hetero atom such as nitrogen atom of a ligand other than the ligand having an oxygen atom to which the metal ion is bonded. The solid may also include a metal ion of the metal ion source coordination-bonded to a nitrogen bond or the like of the phenanthrolinyl group constituting the ligand. That is, all the unreacted ligand and metal ion source generated on synthesis of the metal complex of the present invention do not remain free. Some of them can fall into a coordination bonding state as described above or localize in the vicinity of the metal complex.

### [2] Coordinating Compound

A coordinating compound according to a second embodiment of the present invention is a compound represented by the Formula (1a) to the Formula (3a), comprising one or more phenanthrolinyl groups and a nitrogen-containing fused ring. The coordinating compound can be used for synthesizing the metal complex according to the first embodiment of the present invention and can be a ligand constituting the metal complex.

### [3] Electron Transporting Material

An electron transporting material according to a third embodiment of the present invention (hereinafter, may be referred to as “the electron transporting material of the present invention”) includes the alkali metal complex or alkaline earth metal complex represented by the Formula (1) to the Formula (3) detailed in the first embodiment. The metal complex represented by the Formula (1) to the Formula (3), which can have a wide band gap, is suitable as an electron transporting material.

The structure of “—O-M. . . N-” in the metal complex of the present invention, when used as an electron transporting material, is considered to contribute to imparting solubility in a protic polar solvent such as an alcohol mentioned below and also to contribute to improvement is electron injectability. The phenarthrolinyl group is considered to contribute to improvement in electron transportability and durability. The metal complex of the present invention has a rigid structure in which the ligand portion coordinating to the metal M has a low degree of freedom. Thus, it is considered that an electron transporting material having more excellent durability and a longer lifetime can be given.

The electron transporting material of the present invention preferably contains a dopant because the electron injectability and electron transportability can be improved. As the dopant to be contained in the electron transporting material of the present invention, preferably employed is a dopant having a property of enabling the metal complex of the present invention to be reduced. For example, a compound including an alkali metal or an alkaline earth metal can be used.

One of suitable dopants to be contained in the electron transporting material of the present invention is a metal alkoxide. That is, in the electron transporting material of the present invention, the dopant preferably contains a metal alkoxide.

As the metal alkoxide, a prepared one can be used. Alternatively, a metal alkoxide can be prepared by adding an alkali metal or alkaline earth metal to any alcohol solvent to react the metal with the alcohol solvent.

When a prepared metal alkoxide is employed, a compound represented by the following Formula (7a) and/or (7b) is more suitably used.

### [Formula 37]

R^{H1}—O-M¹ (7a)

R^{H1}—O—M²—O—R^{H2} (7b)

In the Formulas (7a) and (7b), R^{H1} and R^{H2} each independently represent an alkyl group, M¹ represents an alkali metal, and M² represents an alkaline earth metal.

Examples of the alkyl group include linear, branched, or cyclic alkyl groups having 1 to 10 carbon atoms, more preferably 1 to 7 carbon atoms. Specific examples thereof include a methyl group, an ethyl group, a propyl group, an iso-propyl group, a n-butyl group, a sec-butyl group, an iso-butyl group, a tert-butyl group, a n-pentyl group, a 1-methylbutyl group, a 1-ethylpropyl group, a 2-methylbutyl group, a 3-methylbutyl group, a 1,1-dimethylpropyl group, a 1-methyl-2-methylpropyl group, a 2,2-dimethylpropyl group, a n-hexyl group, a 2-methylpentyl group, a 1-methyl-3-methylbutyl group, a 2-ethylbutyl group, a n-heptyl group, a 1-methylhexyl group, a 1-ethylpentyl group, a n-octyl group, a 1-methylheptyl group, a 2-ethylhexyl group, a n-nonyl group, a 3,5,5-trimethylhexyl group, and a n-decyl group. Suitably used among these are a methyl group, an ethyl group, a n-propyl group, an iso-propyl group, a n-butyl group, a sec-butyl group, an iso-butyl group, a tert-butyl group, a n-pentyl group, a n-hexyl group, and the like.

These may be used singly or as a mixture of two or more thereof at any ratio.

A specific example of M¹ is an alkali metal of Li, Na, K, Rb, or Cs, and a specific example of M² is an alkaline earth metal of Be, Mg, Ca, Sr, or Ba. Li is suitably used among these, from the viewpoint of film forming properties and electron transportability.

When an alkali metal or alkaline earth metal is added to an alcohol solvent (alcohol) to prepare a metal alkoxide, the alkali metal or alkaline earth metal is added to the alcohol solvent under an inert gas atmosphere so as to achieve a predetermined concentration, and dissolved under stirring. During dissolution, cooling or heating is carried out as required. When a monohydric alcohol is taken as an example, a reaction represented by the following Reaction Formula (8a) or Reaction Formula (8b) proceeds to thereby prepare a solution in which the metal alkozide is dissolved.

### [Formula 38]

R^{I}—OH+M¹→R^{I}—O-M¹+1/2H₂ (8a)

2R^{I}—OH+M²→R^{I}—O-M²-O—R^{I}+H₂ (8b)

The alcohol is a generic term for compounds having a hydroxyl group (OH group). In the Reaction Formula (8a) or the Reaction Formula (8b), RI corresponds to the portion of the corresponding alcohol solvent excluding the hydroxyl group, M¹ represents an alkali metal, and M² represents an alkaline earth metal.

As the solvent for use in preparation of the metal alkoxide, solvents to be used for a liquid material mentioned below can be used as well. Preferred among these are monohydric alcohols.

Specific examples of the metal alkoxide include sodium methoxide, sodium ethoxide, sodium tert-butoxide, potassium ethoxide, potassium tert-butoxide, lithium n-butoxide, lithium tert-butoxide, and cesium n-heptoxide.

Examples of a suitable dopant to be contained in the electron transporting material of the present invention include one or more selected from the group consisting of a quinolinolate complex, a pyridylphenolate complex, a bipyridylphenolate complex, and an isoquinolinylphenolate complex each having an alkali metal and/or an alkaline earth metal. In the electron transporting material of the present invention, the dopant preferably contains one or more selected from the group consisting of an alkali metal complex of quinolinolate, an alkali metal complex of pyridylphenolate, an alkali metal complex of bipyridylphenolate, and an alkali metal complex of isoquinolinylphenolate.

Specific examples of the quinolinolate complex or phenolate complex include lithium 8-guinolinolate, sodium 8-quinolinolate, cesium 8-quinolinolate, lithium 2-(2-pyridyl)phenolate, sodium 2-(2-pyridyl)phenolate, lithium 2-(2,2′-bipyridin-6-yl)phenolate, and lithium 2-(1-isoquinolinyl)phenolate.

Suitable examples of the dopant to be contained in the electron transporting material of the present invention include an alkali metal hydroxide, an alkali metal salt, an alkaline earth metal hydroxide, and an alkaline earth metal salt. That is, in. the electron transporting material of the present invention, the dopant preferably contains one or more selected from the group consisting of an alkali metal hydroxide, an alkali metal halide, an alkali metal carbonate, an alkali metal hydrogen carbonate, an organic acid salt of an alkali metal having 1 to 9 carbon atoms, an alkaline earth metal hydroxide, an alkaline earth metal halide, an alkaline earth metal carbonate, an alkaline earth metal hydrogen carbonate, and an organic acid salt of an alkaline earth metal having 1 to 9 carbon atoms.

Incorporation of the inorganic compound or organic acid salt enables the electron transportability to be improved and the durability to be improved. The inorganic compound or organic acid salt is likely to dissociate metal ions, resulting in a liquid material for production of organic electroluminescent elements further excellent in higher efficiency and durability and further excellent in productivity.

Specific examples of the inorganic compound or organic acid salt include lithium hydroxide, sodium hydroxide, cesium hydroxide, rubidium, hydroxide, lithium chloride, sodium chloride, potassium chloride, rubidium chloride, cesium chloride, lithium bromide, sodium bromide, potassium bromide, rubidium bromide, cesium bromide, lithium iodide, sodium iodide, potassium iodide, rubidium iodide, cesium iodide, lithium carbonate, sodium carbonate, potassium carbonate, rubidium carbonate, cesium carbonate, lithium hydrogen carbonate, sodium hydrogen carbonate, potassium hydrogen carbonate, rubidium hydrogen carbonate, cesium hydrogen carbonate, lithium acetate, sodium acetate, potassium acetate, rubidium acetate, cesium acetate, lithium formate, sodium formate, potassium formate, rubidium formate, and cesium formate.

As the dopant included in the electron transporting material of the present invention, one of the compounds may be used singly, or any two or more of the compounds may be used in combination. For example, as the dopant included in the electron transporting material of the present invention, the above-described metal alkoxides, complex-based dopants such as an alkali metal complex of guinolinolate, alkali metal hydroxides, alkali metal salts, alkaline earth metal hydroxides, and alkaline earth metal salts may be used singly or may be used in combination.

The proportion of the dopant included in the electron transporting material of the present invention is adjusted in accordance as appropriate with the type and the like of dopant. The proportion of the dopant included in the electron transporting material of the present invention can be 0.1 to 50% by weight, more preferably 1% by weight to 40% by weight, based on the metal complex of the present invention.

The electron transporting material of the present invention may further contain a ligand constituting the metal complex of the present invention, in addition to the metal complex of the present invention. For example, as described above, a solid given by the reaction between a ligand and a metal ion source can be used, as is without purification, as an electron transporting material, and the ligand or metal ion source unreacted may be included in the solid.

### [4]Liquid Material

The invention according to a fourth embodiment of the present invention relates to a liquid material including the electron transporting material according to the third embodiment of the present invention and a solvent (hereinafter, may be referred to as “the liquid material of the present invention).

In the liquid material of the present invention, the solvent is preferably unlikely to cause the organic light emitting layer to be swelled or dissolved. Thereby, when the liquid material is used for production of an organic electroluminescent element, it is possible to prevent alteration or degradation of the organic light emitting layer thin film and excessively thinning of the film thickness. As a result, a liquid material for production of organic electroluminescent elements can be obtained that is further excellent in higher efficiency and durability and further excellent in productivity.

In the liquid material of the present invention, the solvent is preferably a protic polar solvent. Since a lot of emissive materials and hole transport materials are unlikely to be dissolved in a protic polar solvent, use of a protic polar solvent can prevent decrease in the efficiency. As a result, there is given a liquid material further excellent in productivity for use in production of organic electroluminescent elements further excellent in higher efficiency and durability. In the liquid material of the present invention, the solvent is preferably mainly based on an alcoholic solvent. Examples of the proportion of the alcoholic solvent in the solvent of the liquid material include 50% by weight or more, 80% by weight or more, 90% by weight or more, 95% by weight or more, and 100% by weight.

The alcoholic solvent to be used is an alcohol having 1 to 12 carbon atoms, preferably an alcohol having 1 to 10 carbon atoms, more preferably a monohydric or dihydric alcohol having 1 to 7 carbon atoms. Suitably used among these is a monohydric alcohol.

Specific examples of such alcoholic solvents include methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 3,5,5-trimethyl-1-hexanol, 1-decanol, 1-undecanol, 1-dodecanol, allyl alcohol, propargyl alcohol, benzyl alcohol, cyclohexanol, 1-methylcyclohexanol, 2-methylcyc1ohexanol, 3-methvlcyclohexanol, 4-methylcyclohexanol, α-terpineol, abietinol, fusel oil, 1,2-ethanediol, 1,2-propanediol, 1,3-propanediol, 1,2-butanediol, 1,3-butanediol, 1,4-butanediol, 2,3-hutariediol, 1,5-pentanediol, 2-butene-1,4-diol, 2-methyl-2,4-pentanedol, 2-ethyl-1,3-hexanediol, glycerin, 2-ethyl-2-(hydroxymethyl)-1,3-propanediol, 1,2,6-hexanetriol, and

2-methoxyethanol, 2-ethoxyethanol, 2-(methoxyethoxy)ethanol, 2-isopropoxyethanol, 2-butoxyethanol, 2-(isopentyloxy)ethanol, 2-(hexyloxy)ethanol, 2-phenoxyethanol, 2-(benzyloxv)ethanol, furfuryl alcohol, tetrahydrofurfuryl alcohol, diethylene glycol, diethylene glycol monomethyl ether, diethylene glycol monoethyl ether, diethylene glycol monobutyl ether, triethylene glycol, triethylene glycol monomethyl ether, tetraethylene glycol, polyethylene glycol, 1-methoxy-2-propanol, 1-ethoxy-2-propanol, dipropylene glycol, dipropylene glycol monomethyl ether, tripropylene glycol monomethyl ether, diacetone alcohol, 2-chloroethanol, 1-chloro-2-propanol, 3-chloro-1,2-propanediol, 1,3-dichloro-2-propanol, 2,2,2-trifluoroethanol, 3-hydroxypropiononitrile, acetone cyanohydrin, 2-aminoethanol, 2-(dimethylamino)ethanol, 2-(diethylamino)ethanol, diethanolamine, N-butyl diethanolamine, triethanolamine, triisopropanolamine, 2,2′-thiodiethanol, and also

tetrafluoropropanol, peritafluoropropanol, 2,2,2-trifluoroethanol, 2-(perfluorobutyl)ethanol, 3,3,4,4,5,5,6,6,6-nonafluoro-1-hexanol, 2-(perfluorobutyl)ethyl alcohol, 3,3,4,4,5,5,6,6,6-nonafluorohexanol, 1,1,2,2-tetrahydroperfluorohexyl alcohol, 1H, 1H, 2H, 2H-nonafluoro-1-hexanol, 1H, 1H, 2H, 2H-nonafluoro-1-hexanol, 1H,1H-2H,2H-nonafluorohexanol, 1H,1H,2H,2H-perfluorohexan-1-ol, 1H,1H,2H,2H-perfluorohexanol, 3,3,4,4,5,5,6,6,6-nonafluoro-1-hexanol, 2-(perfluorohexyl)ethanol, 3,3,4,4,5,5,6,6,7,7,8,8,8-tridecafluoro-1-octanol, 2-(perfluorohexyl)ethyl alcohol, 3,3,4,4,5,3,6,6,7,7,8,8,8-tridecafluorooctanol, 1,1,2,2-tetrahydroperf1uorooctanol, 1,1,2,2-tetrahydrotridecafluorooctanol, 1H,1H,2H,2H-perflufluoro-1-octanol, 1H,1H,2H,2H-perfluorooctan-1-ol, 1H,1H,2H,2H-perfluorooctanol, 1H,1H,2H,2H-tridecafluoro-n-octanol, 1H,1H,2H,2H-tridecafluorooctanol, 2-(tridecafluorohexyl)ethanol, 3,3,4,4,5,5,6,6,7,7,8,8,8-tridecafluoro-1-ootanol, and perfluorohexylethanol.

More suitably used among these are 1-propanol, 1-butanol, 2-butanol, 1-pentanol, 2-methyl-1-butanoi, 1-hexanol, 1-heptanol, 1-octanol, 2-ethyl-1-hezanol, cyclohexanol, 1-methylcyclohexanol, 2-methylcyclohexanol, 1,2-butanediol, 1,3-butanediol, 1,4-butanediol, 2,3-butanediol, 2-metahoxyethanol, 2-ethoxyethanol, and 2-(methoxyethoxy)ethanol. These may be used singly or as a mixture of two or more thereof at any ratio.

Alcohols having the number of carbon atoms as described above have high dissolvability for the metal complex of the present invention, a metal alkoxide, and the like, and as a result, a liquid material for production of organic electroluminescent elements can be given, the liquid material being further excellent in higher efficiency and durability and further excellent in productivity.

The liquid material of the present invention is desired to contain a metal complex having a structure represented by the Formula (1) to the Formula (3) in an amount of 0.01 to 10% by weight, preferably 0.1 to 5% by weight. When the contert of the metal complex is less than 0.01% by weight, a film thickness required for an organic electroluminescent element may not be formed in contrast, when the contert of the metal complex exceeds 10% by weight, it is difficult to dissolve the metal complex in a solvent.

Although the liquid material of the present invention can be prepared by simultaneously mixing the metal complex of the present invention and the metal alkoxide, a salt of a metal ion, and the like, the liquid material is preferably prepared by mixing a first solution including the metal complex of the present invention and a second solution including the metal alkoxide, salt of a metal ion, and the like.

### [5] Organic Electroluminescent Element

Next, described will be an organic electroluminescent element as a fifth embodiment, which is formed using the electron transporting material of the present invention (third embodiment).

The organic electroluminescent element of the present invention can have an electron transport layer including the electron transporting material of the present invention. That is, the organic electrolumnescent element of the present invention can be an organic electroluminescent element that has an anode, a cathode, and organic compound layers provided between the anode and the cathode and including at least a hole transport layer, a light emitting layer, and an electron transport layer, the electron transport layer including the electron transporting material of the present invention.

A method for producing the organic electroluminescent element of the present invention has a step of constructing the electron transport layer of the organic electroluminescent element by a wet process using the liquid material of the present invention. This production method enables the organic electroluminescent element of the present invention to be produced particularly suitably.

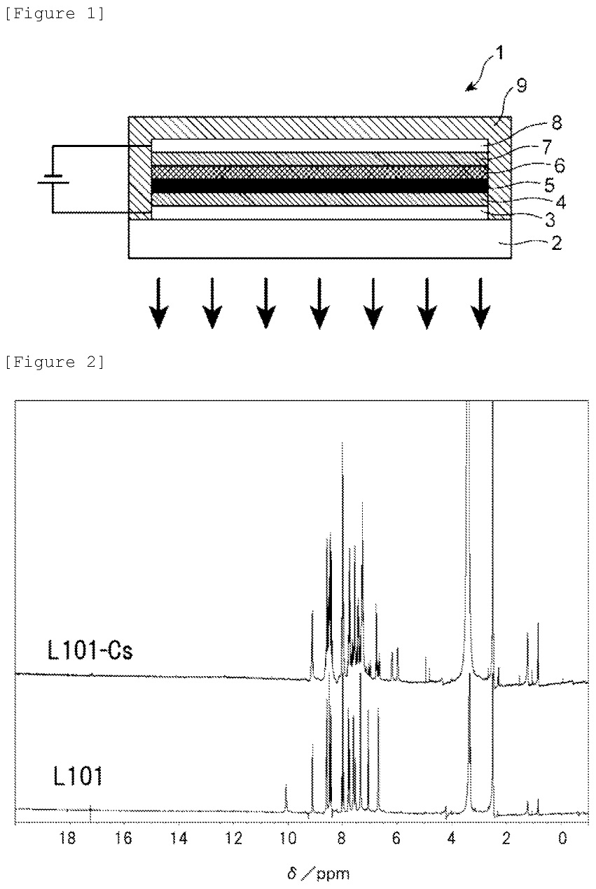

An organic electroluminescent element 1 of the present invention, as illustrated in FIG. 1, is an organic electroluminescent element that has an anode 3, a cathode 8, and a plurality of organic compound layers stacked so as to be interposed between the anode 3 and the cathode 8 (a hole injection layer 4, a hole transport layer 5, a light emitting layer 6, and an electron transport layer 7 in this order from the anode 3 side). The anode 3 is provided on a transparent substrate 2 and entirely sealed by a sealing member 9. The light emitting layer 6 is formed of an organic compound insoluble in an alcoholic solvent. The electron transport layer 7, which is formed such that the surface thereof at the side on which the light emitting layer 6 is opposed to the cathode 8 is contact with the light emitting layer 6, includes one or more electron transporting materials of the present invention soluble in an alcoholic solvent.

The substrate 2 will be a support of the organic electroluminescent element 1. The organic electroluminescent element 1 according to the present embodiment is configured to extract light from the side of the substrate 2 (bottom emission type), and thus, the substrate 2 and the anode 3 each are constituted by a substantially transparent (colorless transparent, colored transparent, or translucent) material. Examples of constituent materials of the substrate 2 include resin materials such as polyethylene terephthalate, polyethylene naphthalate, polypropylene, cycloolefin polymer, polyamide, polyethersulfone, polymethyl methacrylate, polycarbonate, and polyarylate, and glass materials such as quartz glass and soda glass. These may be used singly or in combination of two or more thereof.

The average thickness of the substrate 2 is not particularly limited, and is preferably about 0.1 to 30 mm, more preferably about 0.1 to 10 mm. In the case in which the organic electroluminescent element 1 is configured to extract light from the side opposite to the substrate 2 (top emission type), either of a transparent substrate or an opaque substrate may be used as the substrate 2. Examples of the opaque substrate include substrates constituted by a ceramic material such as alumina, metal substrates, such as stainless steel, including an oxide film (insulating film) formed on the surface thereof, and substrates constituted by a resin material.

The anode 3 is an electrode to inject holes into the hole injection layer 4 mentioned below. The constituent material of this anode 3 to be used is preferably a material having a high work function and excellent in conductivity. Examples of the constituent material of the anode 3 include oxides such as ITO (indium tin oxide), IZO (indium zirconium oxide), In₃O₃, SnO₂, Sb-containing SnO₂, and Al-containing ZnO, Au, Pt, Ag, Cu, or alloys containing these. These may be used singly or in combination of two or more thereof. The average thickness of the anode 3 is not particularly limited, and is preferably about 10 to 200 nm, more preferably about 50 to 150 nm.

Meanwilie, the cathode 8 is an electrode to inject electrons into the electron transport layer 7, provided on the opposite side to the light emitting layer 6, which is in contact with the electron transport layer 7. The constituent material of this cathode 8 to be used is preferably a material having a low work function. Examples of the constituent material of the cathode 8 include Li, Mg, Ca, Sr, La, Ce, Er, Eu, Sc, Y, Yb, Ag, Cu, Al, Cs, Rb, or alloys including these. These may be used singly or in combination of two or more thereof (e.g., a stacked body including a plurality of layers and the like).

Particularly when an alloy is used as the constituent material of the cathode 8, preferably used is an alloy including a stable metal element such as Ag, Al, or Cu, specifically an alloy such as MgAg, AlLi, or CuLi. Use of such an alloy as the constituent material of the cathode 8 enables the electron injection efficiency and stability of the cathode 8 to be improved. The average thickness of the cathode 8 is not particularly limited, and is preferably about 50 to 10000 nm, more preferably about 80 to 500 nm.

In the case of the top emission type, a material having a low work function or an alloy containing the material is formed into a layer having a thickness of about 5 to 20 nm so as to have transmission properties, and a conductive material having high transmission properties such as ITO is further formed thereon into a layer having a thickness of about 100 to 500 nm. The organic electroluminescent element 1 according to the present embodiment is of a bottom emission type, and thus the cathode 8 is not particularly regujred to have light transmission properties.

On the anode 3, the hole injection layer 4 and the hole transport layer 5 are provided. The hole injection layer 4 has a function of receiving holes injected by the anode 3 and transporting the holes to the hole transport layer 5. The hole transport layer 5 has a function of transporting the holes injected by the hole injection layer 4 to the light emitting layer 6. Examples of the hole injection materials and hole transport materials constituting the hole injection layer 4 and the hole transport layer 5, respectively, include a metallophthalocyanine compound or a metal-free phthalocyanine compound such as phthalocyanine, copper phthalocyanine (CuPc), or iron phthalocyanine, polyarylamine, a fuluorene-arylamine copolymer, a fluorine-bithiophene copolymer, poly(N-vinylcarbazole), polyvinylpyrene, polyvinylanthracene, polythiophene, polyalkylthiophene, polyhezylthiophene, poly(p-phenylenevinylene), polythinylenevinylene, pyrene-formaldehyde resin, ethylcarbazole-formaldehyde resin, or derivatives thereof. These may be used singly or in combination of two or more thereof.

The hole injection materials and hole transport materials may be used in admixture with other compounds. Examples of the mixture containing polvthiophene include poly(3,4-ethylenedioxythiophene/styrenesulfonic acid) (PEDOT/PSS). For the hole injection layer 4 and the hole transport layer 5, one or more materials are appropriately selected or combined and employed, from the viewpoint of optimization of the hole injection efficiency and hole transfer efficiency, prevention of reabsorption of light emitted from the light emitting layer 6, heat resistance, and the like depending on the type of material used for the anode 3 and the light emitting layer 6.

For example, for the hole injection layer 4, preferably employed is a material that has a hole conduction level (Ev) slightly different from the work function of the material employed for the anode 3 and has no absorption band in the visible region in order to prevent the reabsorption of the emitted light. For the hole transport layer 5, preferably employed is a material that forms neither exciplex nor charge transfer complex with the constituent material of the light emitting layer 6 and that has singlet excitation energy larger than the exciton energy of the light emitting layer 6, large band gap energy, and a shallow electron conduction level (Ec) in order to prevent move of excitons generated in the light emitting layer 6 and injection of electrons from the light emitting layer 6. Examples of the material suitably employed for the hole injection layer 4 and the hole transport layer 5, when ITO is employed for the anode 3, include poly(3,4-ethylenedioxythiophene/styrenesulfonic acid) (PEDOT/PSS) and poly(N-vinylcarbazole) (PVK), respectively.

In the case where the light emitting layer 6, which is formed. on the hole transport layer 5, is formed by a wet process, a material that is insoluble (neither swells nor dissolves) in the solvent of the liquid material for formation of the light emitting layer is selected for the hole transport material constituting the hole transport layer 5. In the case where the electron transport layer 7 is formed by a wet process, the solvent of the liquid material to be employed for formation of the electron transport layer 7 may cause the hole transport material to swell or dissolve therein. Thus, preferably employed is a material that is insoluble in the solvent of the liquid material for formation of the electron transport layer.

The average thickness of the hole injection layer 4 is not particularly limited, and is preferably about 10 to 150 nm, more preferably about 20 to 100 nm. The average thickness of the hole transport layer 5 is not particularly limited, and is preferably about 10 to 150 nm, more preferably about 15 to 50 nm.

The light emitting layer 6 is provided on the hole transport layer 5, that is, adjacent to the surface on the opposite side to the hole injection layer 4. To the light emitting layer 6, electrons are supplied (injected) via the electron transport layer 7 from the cathode 8, and holes are supplied (injected) from the hole transport layer 5. Then, inside the light emitting layer 6, holes recombine with electrons. Excitons are generated with energy released on the recombination, and when the excitons return to their ground state, energy (fluorescence or phosphorescence) is released (emission).

Examples of the constituent material of the light emitting layer 6 include benzene compounds such as 1,3,5-tris[(3-phenyl-6-tri-fluoromethyl)quinoxalin-2-yl]benzene (TPQ1) and 1,3,5-tris[{3-(4-tert-butylphenyl)-6-trisfluoromethyl}quinoxalin-2-yl]benzene (TPQ2), low-molecular-weight materials such as tris(8-quinolinolato)aluminum(III) (Alq3) and fac-tris(2-phenylpyridine)iridium (Ir(ppy)3), low-molecular-weight or polymeric materials such as oxadiazole material, triazole material, and carbazole material, and polymeric materials such as polyfluorene material, polyphenylene vinylene material, polypyrrole material, polyacetylene material, and polyaniline material. These may be used singly or in combination of two or more thereof.

The light emitting layer 6 may be constituted by a single material, or a plurality of materials may be combined in accordance with the luminescent color and the like. Also may be employed a two-component system including a guest material responsible for light emission and a host material responsible for transport of electrons and holes. When a host-guest two-component system is employed, in the light emitting layer 6, the concentration of the guest material is generally 0.1 to 1% by weight with respect to the host material.

In the case where the electron transport layer 7, which is formed on the light emitting layer 6, is formed by a wet process, a material insoluble (neither swells nor dissolves) in the solvent of the liquid material for formation of the electron transport layer is selected for the constituent material of the light emitting layer 6. The electron transport layer including the electron transporting material of the present invention can be constructed using a protic polar solvent (in particular, alcohol). Thus, the light emitting layer 6 is preferably a layer insoluble in a protic polar solvent, more preferably a layer insoluble in alcohol.

The average thickness of the light emitting layer 6 is not particularly limited, and is preferably about 10 to 150 nm, more preferably about 20 to 100 nm.

The electron transport layer 7 is provided between the light emitting layer 6 and the cathode 8. This electron transport layer 7 has a function of transporting electrons injected from the cathode 8 to the light emitting layer 6. As a constituent material of the electron transport layer 7, the electron transporting material according to the third embodiment of the present invention is used.

The average thickness of the electron transport layer is not particularly limited, and is preferably about 1 to 100 nm, more preferably about 1 to 50 nm, further preferably about 5 to 50 nm.

In the organic electroluminescent element 1, the cathode 8 is provided on the electron transport layer 7, that is, adjacent to the surface on the opposite side to the light emitting layer 6. Incorporation of the electron transport layer 7, for which the electron transporting material of the present invention is employed, enables the emission efficiency of the light emitting layer to be improved and the degree of freedom of optical design to be enhanced, even when the cathode 8 is formed directly on the electron transport layer 7 without provision of an electron injection layer in which an unstable compound such as NaF or LiF is used.

Next, the sealing member 9, which is provided so as to cover the organic electroluminescent element 1 (the anode 3, the hole injection layer 4, the hole transport layer 5, the emissive layer 6, the electron transport layer 7, and the cathode 8), has a function of hermetically sealing the layers to block oxygen and moisture. When the sealing member 9 is provided, effects can be achieved such as improvement in the reliability and prevention of alteration or degradation (improvement in the durability) of the organic electroluminescent element 1.

Examples of the constituent material of the sealing member 9 include Al, Au, Cr, Nb, Ta, Ti, or alloys including these, silicon oxide, and various resin materials. In the case in which a material having electrical conductivity is employed as the constituent material of the sealing member 9, an insulation film is preferably provided as required between the sealing member 9 and the organic electroluminescent element 1 in order to prevent a short circuit. The sealing member 9 may be formed in a flat shape and disposed to face the substrate 2. Then, a sealing material of a thermosetting resin or the like may be used to seal between the sealing member 9 and the substrate 2.

The organic electroluminescent element of the present invention is not limited to the organic electroluminescent element 1.

In the organic electroluminescent element 1, the hole injection layer 4 and the hole transport layer 5 are formed as two separate layers between the anode 3 and the light emitting layer 6. However, as required, the layers may be formed as a single hole transport layer that injects holes from the anode 3 and transports holes to the light emitting layer 6 or may have a structure in which three or more layers having the same or different compositions with one another are stacked. The light emitting layer is a single layer but may have a structure in which a plurality of layers having the same or different compositions are stacked. For example, the light emitting layer may have a structure in which a plurality of light emitting layers each having a different composition in accordance with a color desired to be emitted or the like. The electron transport layer also may have the same or different compositions with each other.

The organic electroluminescent element of the present invention may have further a layer, other than the hole injection layer, the hole transport layer, the light emitting layer, and the electron transport layer, between the anode and the cathode, or may have a structure in which an electron injection layer made of NaF, LiF, or the like is provided between the cathode 8 and the electron transport layer 7.

The organic electroluminescent element 1 can be formed by, for example, a production method of constructing an organic compound layer by a wet process, as follows.

First, a substrate 2 is provided, and an anode 3 is formed on this substrate 2. The anode 3 may be formed by, for example, chemical vapor deposition (CVD) such as plasma CVD, thermal CVD, or laser CVD; dry plating such as vacuum deposition, sputtering, or ion plating; wet plating such as electrolytic plating, immersion plating, or electroless plating; thermal spray; sol-gel method; MOD process; or bonding of metal foil or the like.

Then, a hole injection layer 4 and a hole transport layer 5 are sequentially formed on the anode 3.

The hole injection layer 4 and the hole transport layer 5 may be formed by, for example, by supplying a liquid material for forming a hole injection layer prepared by dissolving or dispersing the hole injection material in a solvent or dispersion medium on the anode 3 followed by drying (removing the solvent or dispersion medium), and then supplying a liquid material for forming a hole transport layer prepared by dissolving or dispersing the hole transport material in a solvent or dispersion medium on the hole injection layer 4 followed by drying. Examples of the method of supplying the liquid material for forming a hole injection layer or the liquid material for forming a hole transport layer include coating methods such as a spin coating method, a casting method, a microgravure coating method, a gravure coating method, a bar coating method, a roll coating method, a wire bar coating method, a dip coating method, a spray coating method, a screen printing method, a flexoprinting method, an offset printing method, and an inkjet printing method. Use of such a coating method may relatively factate formation of the hole injection layer 4 and the hole transport layer 5.

Examples of solvents and dispersion media used for preparing the liquid material for forming a hole injection layer and the liquid material for forming a hole transport layer include inorganic solvents such as nitric acid, sulfuric acid, ammonia, hydrogen peroxide, water, carbon disulfide, carbon tetrachloride, and ethylene carbonate; ketone solvents such as methylethyl ketone (MEK), acetone, diethyl ketone, methylisobutyl ketone (MIBK), methylisopropyl ketone (MIPK), and cyclohexanone; alcoholic solvents such as methanol, ethanol, isopropanol, ethylene glycol, diethylene glycol (DEG), and glycerin; ether solvents such as diethyl ether, diisopropyl ether, 1,2-dimethoxyethane (DME), 1,4-dioxane, tetrahydrofuran (THF), tetrahydropyran (THP), anisole, diethylene glycol dimethyl ether (diglyme), and diethylene glycol ethyl ether (carbitol); cellosolve solvents such as methyl cellosolve, ethyl cellosolve, and phenyl cellosolve; aliphatic hydrocarbon solvents such as hexane, pentane, heptane, and cyclohexane; aromatic hydrocarbon solvents such as toluene, xylene, and benzene; aromatic heterocyclic compound solvents such as pyridine, pyradine, furan, pyrrole, thiophene, and methylpyrrolidone; amide solvents such as N,N-dimethylformamide (DMF) and N,N-dimethylacetoamide (DMA); halogen compound solvents such as chlorobenzene, dichloromethane, chloroform, and 1,2-dichlroethane; ester solvents such as ethyl acetate, methyl acetate, and ethyl formate; sulfux compound solvents such as dimethylsulfoxide (DMSO) and sulfolane; nitrile solvents such as acetonitrile, propionitrile, and acrylonitrile; various organic solvents including organic acid solvents such as formic acid, acetic acid, trichloroacetic acid, and trifluoroacetic acid; or mixture solvents including these.

Drying may be carried out, for example, by standing at atmospheric pressure or under reduced pressure, heating, blowing by inert gas, or the like.

In advance of the step, the top surface of the anode 3 may be subjected to oxygen plasma treatment. This enables the top surface of the anode 3 to be provided with affinity, to liquid, organic matter attached on the top surface of the anode 3 to be removed (washed), the work function of the anode 3 in the vicinity of the top surface to be adjusted, and the like.

Preferred conditions for the oxygen plasma treatment here include, for example, a plasma power of about 100 to 800 W, a flow rate of oxygen gas of about 50 to 100 mL/min, a transport speed of the member to be treated (the anode 3) of about 0.5 to 10 mm/sec, and a temperature of the substrate 2 of about 70 to 90° C.

Then, a light emitting layer 6 is formed on the hole transport layer 5 (on the side of one surface of the anode 3).

The light emitting layer 6 may be formed by, for example, supplying a liquid material for forming a light emitting layer prepared by dissolving or dispersing the constituent material of the light emitting layer 6 in a solvent or dispersion medium on the hole transport layer 5 followed by drying (removing the solvent or dispersion medium). The methods of supplying and of drying the liquid material for forming a light emitting layer are the same as those described in the formation of the hole injection layer 4 and hole transport layer 5.

As the solvent or dispersion medium to be employed for preparation of the liquid material for forming the light emitting layer, solvents the same as those described in formation of the hole injection layer 4 and hole transport layer 5 may be used. In accordance with the formed hole transport layer 5, a solvent which does not dissolve the hole transport layer 5 is selected.

Then, an electron transport layer 7 is formed on the light emitting layer 6 by the following steps, for example.

### (a) First Step

First, a liquid material for for an electron transport layer that includes a metal complex represented by the Formulas (1) to (3) and, as required, a dopant such as a metal alkoxide is prepared.

The solvent to be employed for preparation of the liquid material for forming an electron transport layer is preferably a solvent in which. the constituent material of the light emitting layer 6 is unlikely to swell or dissolve, more preferably a solvent in which the constituent material is insoluble. This can prevent alteration or degradation of the emissive material and reduction is the film thickness due to dissolution of the light emitting layer 6. As a result, decrease in the emission efficiency of the organic electroluminescent element 1 can be prevented. The liquid material for forming an electron transport layer may cause the constituent material of the hole transport layer 5 to swell or dissolve. Thus, the solvent is preferably a solvent in which the constituent material of the light emitting layer 5 is unlikely to swell or dissolve, more preferably a solvent in which the constituent material is insoluble. Many of materials constituting the hole transport layer 5 or the light emitting layer 6 are unlikely to dissolve in a protic polar solvent, particularly alcohol. Thus, the alcoholic solvents mentioned above, preferably, alcohols having 1 to 10 carbon atoms are suitably used as the solvent. This enables decrease in the emission efficiency to be prevented and the organic electroluminescent element 1 to be produced with favorable productivity.

### (b) Second Step

Next, the liquid material prepared is supplied on the light emitting layer 6 and then dried (removing the solvent). This enables the electron transport layer 7 to be given, which contains the metal complex. represented by the Formula (1) to the Formula (3). The methods of supplying and of drying the liquid material for forming an electron transport layer are the same as those described in the formation of the hole injection layer 4 and hole transport layer 5.

Although the first step and the second step may be sequentially carred out, the liquid material for forming an electron transport layer may be prepared in advance without sequentially carrying out the first step and the second step. The electron transport layer 7 may be constructed by supplying the liquid material for forming an electron transport layer prepared in advance on the light emitting layer 6 followed by drying (removing the solvent).

Then, a cathode 8 is formed on the electron transport layer 7.

The cathode 8 may be formed by, for example, a vacuum deposition method, a sputtering method, bonding of metal foil, or application of metal fine particle ink followed by sintering.

Finally, the sealing member 9 is placed over the organic electroluminescent element 1 such that the organic electroluminescent element 1 is covered with the sealing member 9, and bonded to the substrate 2. Through the steps described above, the organic electroluminescent element 1 is given.

According to the production method, formation of the organic compound layers (the hole injection layer 4, the hole transport layer 5, the light emitting layer 6, and the electron transport layer 7) and formation of the cathode 8, also, if metal fine particle ink is used, do not require large-scale equipment such as vacuum equipment. Thus, the time and cost required for the production of the organic electroluminescent element 1 may be reduced. Use of an inkjet method (droplet discharging method) facilitates production of large elements and printing in multiple colors.

Although the method for producing the organic electroluminescent element 1 has been described as production of the hole injection layer 4, the hole transport layer 5, and the light emitting layer 6 by a liquid phase process, in the method for producing an organic electroluminescent element of the present invention, some or all of these layers may be formed by a gas phase process such as a vacuum deposition method, in accordance with the type of hole injection material, hole transport material, and emissive material to be employed.

The organic electroluminescent element of the present invention may be used as a light source or the like. A plurality of the organic electroluminescent element of the present invention may be arranged in a matrix to constitute a display unit.

The drive system of such a display unit is not particularly limited, and may be either an active matrix system or a passive matrix system.

The electric energy supply for the organic electroluminescent element of the present invention is mainly direct current, but pulse current or alternative current also can be used. Although values of the current and voltage are not particularly limited, in consideration of the power consumption and lifetime of the element, the maximum luminance should be achieved with energy as low as possible.

The “matrix” constituting the display unit refers to pixels for display arranged in a grid, displaying characters and images by means of sets of pixels. The shape and size of the pixels are determined in accordance with the applications. For example, for displaying images and characters on a personal computer, a monitor, or a television, square pixels having a side of 300 μm or less are usually employed. In the case of a large-scale display such as a display panel, pixels having a side in the order of a few mm are employed. For monochrome display, it only required that pixels of the same color are arranged, whereas, for color display, red, green and blue pixels are to be arranged for display. Typical examples of the matrix in this case includes a delta type and a stripe type. The driving system of this matrix may be either a passive matrix system or an active matrix system. Although the former advantageously has a simple structure, the latter active matrix system is more excellent in some cases, in consideration of the operating characteristics. Thus, the systems are required to be chosen properly in accordance with the application.

The organic electroluminescent element may be a segment type display unit. In the “segment type”, a pattern of a predetermined shape is formed so as to display predetermined information, and a predetermined region is caused to emit light. Examples thereof include time or temperature displays in digital clocks or thermometers, operating status displays of audio devices, electromagnetic cookers, and the like, and instrument panel displays of automobiles. Both the matrix display and the segment type display may coexist on the same panel.

The organic electroluminescent element of the present invention may be a backlight for enhancing the visibility of non-self-luminous display units, which backlight is used in a liquid crystal display unit, a clock, an audio device, an automobile panel, a display panel, a sign board, or the like. The backlight for use particularly in liquid crystal display units, especial in personal computers, in which reduction in the thickness poses a problem, can be made thinner and lighter than conventional ones formed of a fluorescent lamp or light guiding panel.

### EXAMPLES

Compounds were confirmed by thin layer chromatography and FAB MS or ASAP-TOF-MS. FAB MS was measured using JMS700 manufactured by JEOL Ltd. ASAP-TOF-MS was measured using LCT Premire XE manufactured by Waters Corporation.

NMR (400 MHz) of some ligands and complexes were measured using JNM-LA400 manufactured by JEOL Ltd. by use of DMSO-d6 as the deuterated solvent.

As silica gel C300 used for column chromatography, Wakosil C300 (C300) manufactured by Wako Pure Chemical industries, Ltd. and Chromatorex NH₂ (NH₂) manufactured by Fuji Silysia Chemical, Ltd. were employed.

### [1] Synthesis of Metal Complex

### [A] Metal Complex Represented by Formula (1)

### [A-1] Synthesis of L101-M Complex (M=Cs, Rb)

### [A-1-1] Synthesis of Ligand L101

### (1-1-1) Synthesis of Intermediate: Synthesis of N-(3-Chlorophenyl)-3-Methoxy-2-Nitrosoaniline

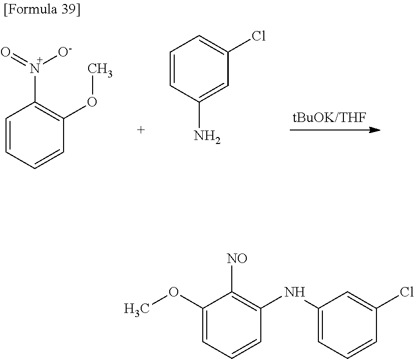

To a KOtBu (10 g, 180 mmol)-THF (80 mL) solution cooled to −70° C., 3-chloroaniline (3.16 mL, 30 mmol) was added dropwise, and the mixture was stirred for 30 minutes. A 2-nitroanisole (3.1 mL, 30 mmol)-THF (20 mL) solution was added dropwise, and the mixture was stirred at −50° C. After two hours, a saturated ammonium chloride aqueous solution was added thereto to stop the reaction. After completion of the reaction, the mixture was subjected to extraction with dichloromethane. The organic layer was dried over magnesium sulfate and then concentrated under reduced pressure. The residue was purified by column chromatography (C300, heptane:dichloromethane) to give N-(3-chlorophenyl)-3-methoxy-2-nitrosoaniline (6.28 g, 79.8%).

ASAP−TOF−MS m/z=263 ([M+1]⁺)

(1-1-2) Synthesis of Intermediate: 1-(3-chlorophenyl)-4-methoxy-2-phenyl-1H-benzimidazole

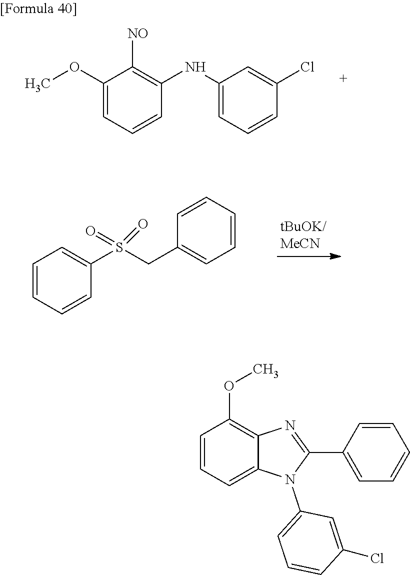

N-(3-Chlorophenyl)-3-methoxy-2-nitrosoaniline (3.15 g, 14.4 mmol) obtained in (1-1-1), benzyl phenyl sulfone (3.35 g, 14.4 mmol), and KOtBu (13.5 g, 120 mmol) were added to 144 mL of acetonitrile, and the mixture was stirred at room temperature for 16 hours. After completion of the reaction, the reaction solution was poured into water, and the mixture was concentrated under reduced pressure. Subsequently, the mixture was subjected to extraction with dichloromethane. The organic layer was washed with water, then dried over magnesium sulfate, and concentrated under reduced pressure. The residue was purified. by column chromatography (C300, heptane:dichloromethane) to give 1-(3-chlorophenyl)-4-methoxy-2-phenyl-1H-benzimidazole (1.68 g, 41.8%).

(1-1-3) Synthesis of intermediate: 1-[3-(4,4,5,5-tetramethyl-1,3,2-dioxaborolan-2-yl)phenyl]-4-methoxy-2-phenyl-1H-benzimidazole

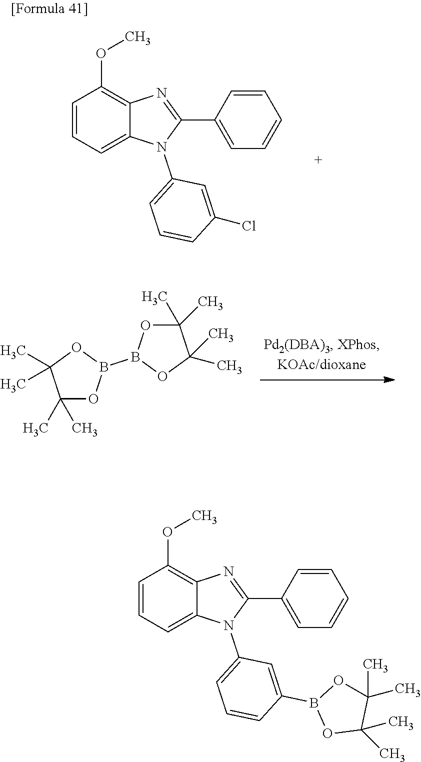

1-(3-Chlorophenyl)-4-methoxy-2-phenyl-1H-benzimidazole (1.68 g, 5.02 mmol) obtained in (1-1-2), bis(pinacolato)diboron (1.39 g, 5.48 mmol), tris(dibenzylideneacetone)dipalladium (0.17 g, 0.19 mmol), XPhos (0.22 p, 0.46 mmol), and potassium acetate (1.47 g, 15 mmol) were added to 18.8 of dioxane, and the mixture was degassed and then stirred at 80° C. for 16 hours. After completion of the reaction, the mixture was concentrated under reduced pressure, water was added thereto, and the mixture was subjected to extraction with dichloromethane. The organic layer was dried over magnesium sulfate and. concentrated under reduced pressure. The residue was purified by column chromatography (C300, heptane:dichloromethane) to give 1-[3-(4,4,5,5-tetramethyl-1,3,2-dioxaborolan-2-yl)phenyl]-4-methoxy-2-phenyl-1H-benzimidazole (1.65 g, 77.1%).

(1-1-4) Synthesis of intermediate: 1-[3-(1,10-phenanthrolin-2-yl )phenyl]-4-methoxy-2-phenyl-1H-benzimidazole

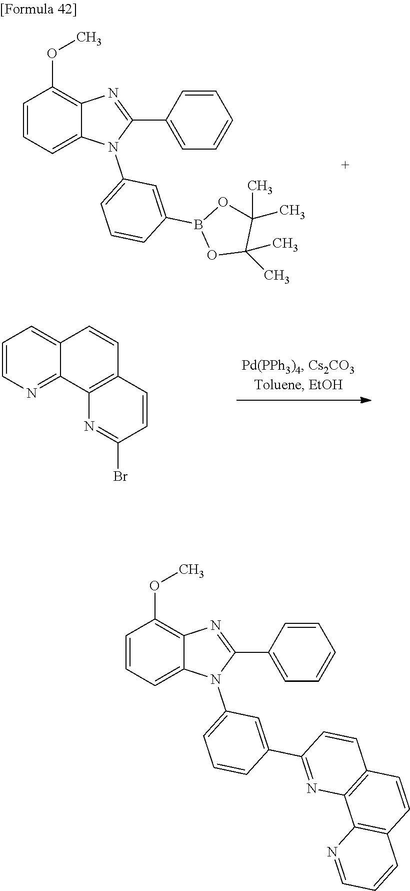 [SMILES: COc1cccc2c1nc(-c1ccccc1)n2-c1cccc(B2OC(C)(C)C(C)(C)O2)c1 ; Brc1ccc2ccc3cccnc3c2n1 ; COc1cccc2c1nc(-c1ccccc1)n2-c1cccc(-c2ccc3ccc4cccnc4c3n2)c1]

1-[3-(4,4,5,5-Tetramethyl-1,3,2-dioxaboralen-2-yl)phenyl]-4-methoxy-2-phenyl-1H-benzimidazole (1.8 g, 4.22 mmol) given by conducting (1-1-3) several times, 2-bromo-1,10-phenanthroline (1.09 g, 4.22 mmol), tetrakis(triphenylphosphine)palladium (0.26 g, 0.23 mmol), and cesium carbonate (4.56 g, 14 mmol) were added to 27 mL of toluene, 4.5 mL of water, and 2.8 mL of ethanol, and the mixture was stirred at 100° C. for 16 hours. After completion of the reaction, the water was added thereto, and the mixture was subjected. to extraction with dichloromethane. The organic layer was dried over magnesium sulfate and then concentrated under reduced pressure. The concentrate was purified by column chromatography (NH₂, heptane:dichloromethane) to give 1-[3-(1, 10-phenanthrolin-2-yl)phenyl]-4-methoxy-2-phenyl-1H-benzimidazoie (1.12 q, 55%).

(1-1-5) Synthesis of Ligand L101: 1-[3-(1, 10-phenanthrolin-2-yl)phenyl]-2-phenyl-1H-benzmidazo1-4-ol

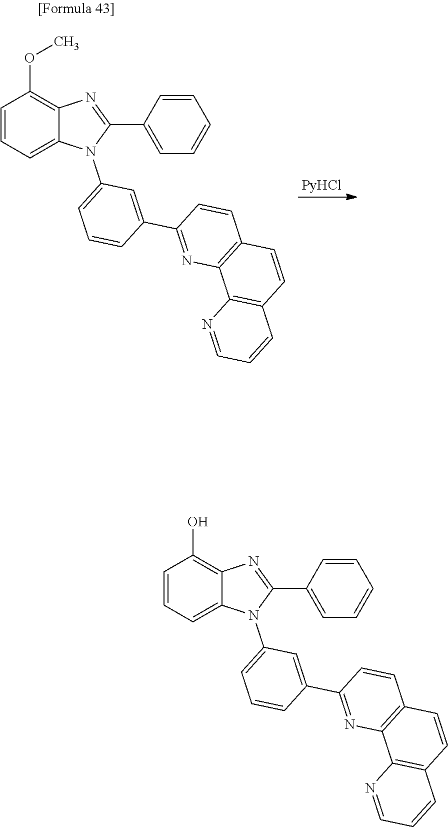 [SMILES: COc1cccc2c1nc(-c1ccccc1)n2-c1cccc(-c2ccc3ccc4cccnc4c3n2)c1 ; Oc1cccc2c1nc(-c1ccccc1)n2-c1cccc(-c2ccc3ccc4cccnc4c3n2)c1]

To 1-[3-(1, 10-phenanthrolin-2-yl)phenyl]-4-methoxy-2-phenyl-1H-benzimidazole (1.12 g, 2.34 mmol) obtained in (1-1-4), pyridine hydrochloride (6.3 g, 54.6 mmol) was added, and the mixture was stirred at 200° C. for 16 hours. After completion of the reaction, water was added thereto, and the insoluble was filtered off to give 1-[3-(1, 10-phenanthrolin-2-yl)phenyl]-2-phenyl-1H-benzmidazol-4-ol (L101) (1.00 g, 92.6%).

### [A-1-2] Synthesis of Complex: Synthesis of L101-Cs

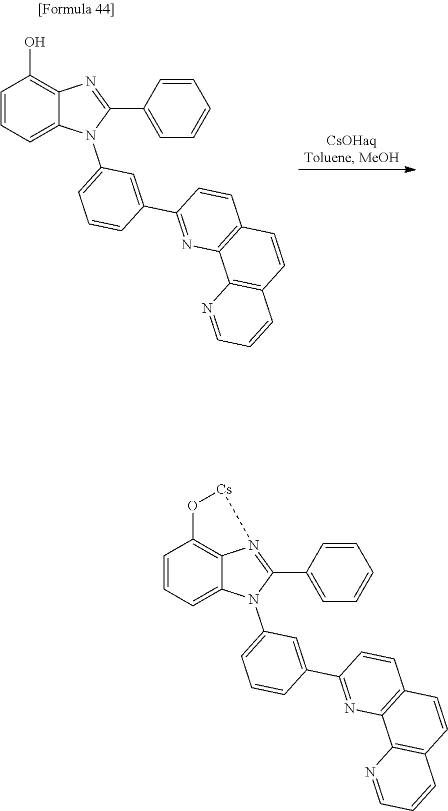

To 8 mL of L101 (0.2 g, 0.431 mmol) obtained in [A-1-1]—toluene, a 50% cesium hydroxide aqueous solution (0.075 ml, 0.431 mmol)—methanol (3 mL) solution was added dropwise, and the mixture was stirred at room temperature for 16 hours. After completion of the reaction, the mixture was concentrated under reduced pressure, heptane was added thereto, and then the mixture was dried to solid to give L101-Cs (0.23 g, 89.6%).

FAB−MS m/z=597 ([M+1]⁺)

The NMR of the complex obtained is also shown in FIG. 2, together with the NMR of L101.

### [A-1-3] Synthesis of Complex: Synthesis of L101-Rb

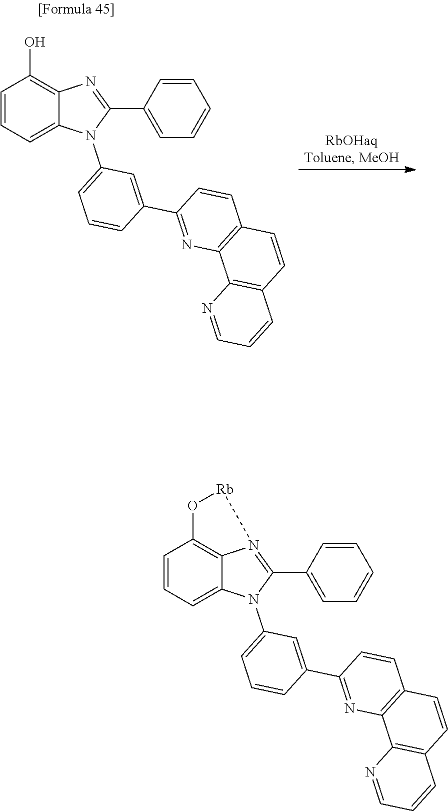

To 8 mL of L101 (0.2 g, 0.431 mmol) obtained in [A-1-1]—toluene, a 50% rubidium hydroxide aqueous solution (0.051 m1, 0.431 mmol)—methanol (3 mL) solution was added dropwise, and the mixture was stirred at room temperature for 16 hours. After completion of the reaction, the mixture was concentrated under reduced pressure, heptane was added thereto, and then mixture was dried to solid to give L101-Rb (0.2 g, 84.5%).

FAB−MS m/z=548 ([M+1]⁺)

### [A-2] Synthesis of L102-M Complex (M=Cs)

### [A-2-1] Synthesis of Ligand L102

(1-2-1) Synthesis of Intermediate: Synthesis of 1-(1, 10-Phenanthrolin-2-yl)Amino-3-Methoxy-2-Nitrobenzene

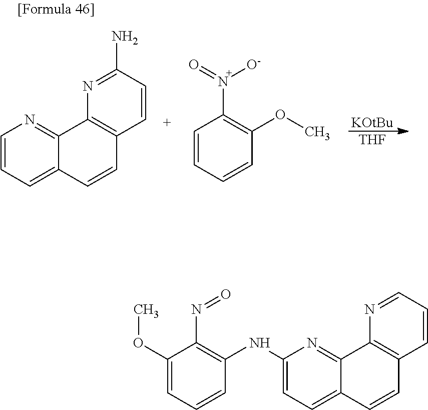

To a KOtBu (5 g, 44.6 mmol)—THF (40 mL) solution cooled to −70° C., 2-amino-1,10-phenanthroline (3 g, 15.4 mmol) was added dropwise, and the mixture was stirred for 30 minutes. A 2-nitroanisole (1.84 m1, 15 mmol)—THF (10 mL) solution was added thereto, and the mixture was stirred at −50° C. After two hours, a saturated ammonium chloride aqueous solution was added thereto to stop the reaction. After completion of the reaction, the mixture was subjected to extraction with dichloromethane. The organic layer was dried over magnesium sulfate and then concentrated under reduced. pressure. The residue was purified by column chromatography (NH₂, heptane:dichloromethane) to give 1-(1, 10-phenanthrolin-2-yl)amino-3-methoxy-2-nitrobenzene (4.47 g, 90.3%).

(1-2-2) Synthesis of intermediate: synthesis of 4-methoxy-2-phenyl-1-(1, 10-phenanthrolin-2-yl)-1H-benzimidazole

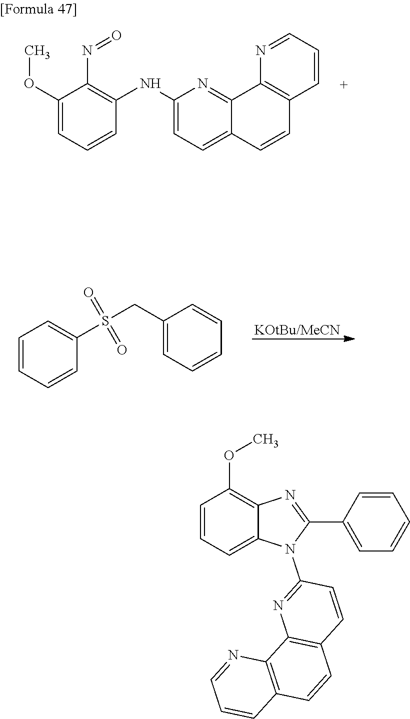 [SMILES: COc1cccc(Nc2ccc3ccc4cccnc4c3n2)c1N=O ; O=S(=O)(Cc1ccccc1)c1ccccc1 ; "KOtBu/MeCN" ; COc1cccc2c1nc(-c1ccccc1)n2-c1ccc2ccc3cccnc3c2n1]

1-(1, 10-Phenanthrolin-2-yl)amino-3-methoxy-2-nitrobenzene 3.15 g, 14.4 mmol) obtained in (1-2-1), benzyl phenyl sulfone (3.35 g, 14.4 mmol), and KOtbu (13.5 g, 120 mmol) were added to 144 mL of acetonitrile, and the mixture was stirred at room temperature for 16 hours. After completion of the reaction, the reaction solution was poured into water., and the mixture was concentrated under reduced pressure. Subsequently, the mixture was subjected to extraction. with dichloromethane. The organic layer was washed with water, then dried over magnesium sulfate, and concentrated under reduced. pressure. The residue was purified by column chromatography (NH₂, heptane:dichloromethane) to give 4-methoxy-2-phenyl-1-(1, 10-phenanthrolin-2-y1)-1H-benzimidazole (2.42 g, 41.8%).

(1-2-3) Synthesis of Ligand L102: synthesis of 4-hydroxy-2-phenyl-1-(1, 10-phenanthrolin-2-yl)-1H-benzimidazole

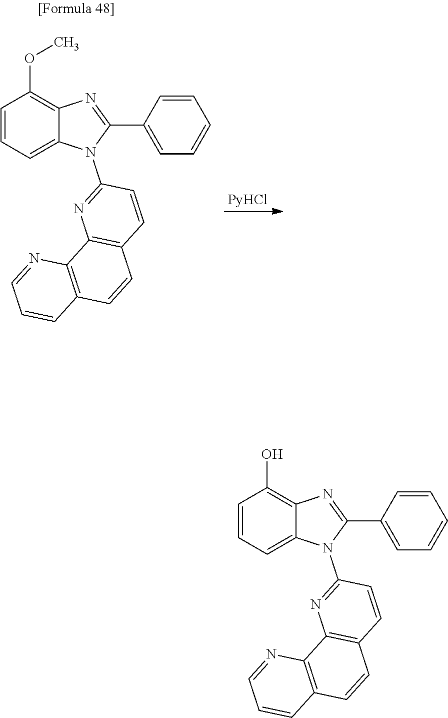 [SMILES: COc1cccc2c1nc(-c1ccccc1)n2-c1ccc2ccc3cccnc3c2n1 ; Oc1cccc2c1nc(-c1ccccc1)n2-c1ccc2ccc3cccnc3c2n1]

To 4-methoxy-2-phenyl-1-(1, 10-phenanthrolin-2-yl)-1H-benzimidazole (1.12 g, 2.34 mmol) obtained in (1-2-2), pyridine hydrochloride (6.3 g, 54.6 mmol) was added, and the mixture was stirred at 200° C. for 16 hours. After completion of the reaction, water was added thereto, and the insoluble was filtered off to give 4-hydroxy-2-phenyl-1-(1, 10-phenanthrolin-2-yl)-1H-benzimidazole (L102) (1.00 g, 76.6%).

[A-2-2] Synthesis of Complex: Synthesis of L102-Cs complex

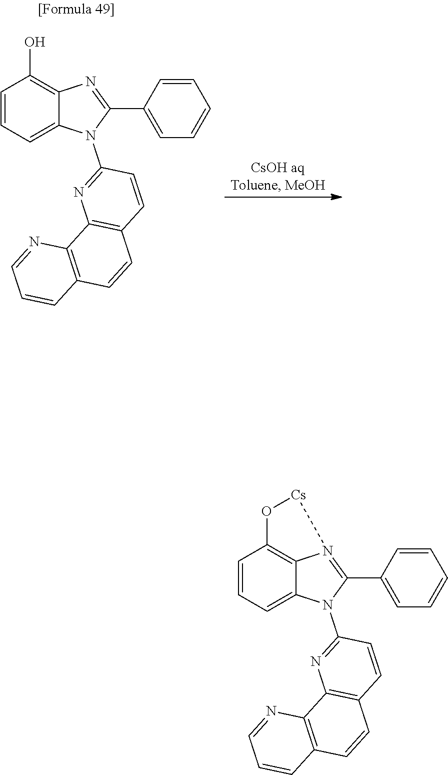

To a L102 (0.100 g, 0.257 mmol) obtained in [A-2-1]—toluene (4.8 mL) suspension, a 50% cesium hydroxide aqueous solution (0.045 mL, 0.257 mmol)—methanol (1.92 mL) solution was added dropwise, and the mixture was stirred at room temperature for one hour. After completion of the reaction, the mixture was concentrated under reduced pressure, heptane was added thereto, and then the mixture was dried to solid to give L102-Cs (0.110 g, 82.7%).

### [A-2-3] Synthesis of Composition Containing L102-Cs (1a)

A dried solid (0.104 g, 89.0%) was given by the same operation as in [A-2-2] except that the amount of the 50% cesium hydroxide aqueous solution was changed from 0.045 mL (0.257 mmol) to 0.022 mL (0.128 mmol) such that L102 would be in excess relative to the metal ion source (50% cesium hydroxide aqueous solution). The dried solid given was taken as a composition containing L102-Cs (1a).

### [A-2-4] Synthesis of Composition Containing 1102-Cs (1b)

A dried solid (0.119 g, 76.2%) was gives by the same operation as in [A-2-2] except that the amount of the 50% cesium hydroxide aqueous solution was changed from 0.045 mL (0.257 mmol) to 0.058 (0.333 mmol) such. that the metal ion source (50% cesium hydroxide aqueous solution) would be in excess relative to L102. The dried solid given was taken as a composition containing L102-Cs (1b).

### [A-3] Synthesis of L103-M Complex (M=Cs, Li)

### [A-3-1] Synthesis of Ligand L103

(1-3-1) Synthesis of Intermediate: 3-anilino-4-chloro-2-Nitrosoanisole

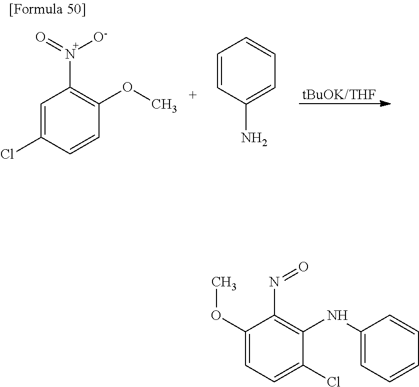

To a KOtBu (30 g, 267 mmol)—THF (240 mL) solution cooled to −70° C., aniline (6.12 mL, 90 mmol) was added dropwise, and the mixture was stirred for 30 minutes. A 4-chloro-2-nitroanisole (16.9 g, 90 mmol)—THF (60 mL) solution was added dropwise, and the mixture was stirred at −50° C. After two hours, a saturated ammonium chloride aqueous solution was added to stop the reaction. After completion of the reaction, the mixture was subjected to extraction with dichloromethane. The organic layer was dried over magnesium sulfate and then concentrated. under reduced pressure. The residue was purified by column chromatography (C300, heptane:dichloromethane) to give N-3-anilino-4-chloro-2-nitrosoanisole (12.7 g, 53.6%).

(1-3-2) Synthesis of Intermediate: Synthesis of 7-chloro-4-Methoxy-1,2-Diphenyl-1H-Benzimidazole

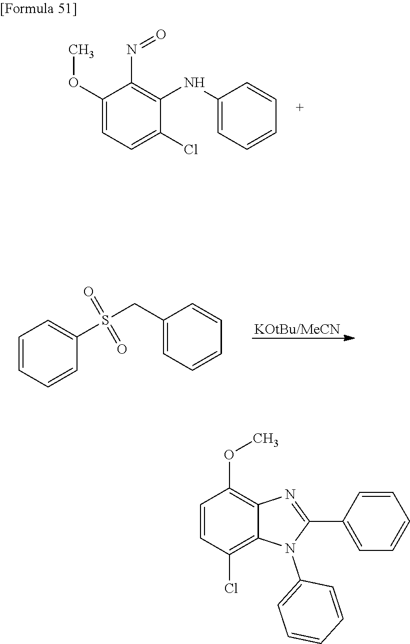 [SMILES: COc1ccc(Cl)c(Nc2ccccc2)c1N=O ; O=S(=O)(Cc1ccccc1)c1ccccc1 ; "KOtBu/MeCN" ; COc1ccc(Cl)c2c1nc(-c1ccccc1)n2-c1ccccc1]

3-Anilino-4-chloro-2-nitrosoanisoie (4.0 g, 15.2 mmol) obtained in (1-3-1), benzyl phenyl sulfone (4.35 g, 18.7 mmol), and KOtBu (17.5 g, 156 mmol) were added to 187 mL of acetonitrile, and the mixture was stirred at room temperature for 16 hours. After completion of the reaction, the reaction solution was poured into water, and the mixture was concentrated under reduced pressure. Subsequently, the mixture was subjected to extraction with dichloromethane. The organic layer was washed with water, then dried over magnesium sulfate, and concentrated under reduced pressure. The residue was purified by column chromatography (C300, heptane:dichloromethane) to give 7-chloro-4-methoxy-1,2-diphenyl-1H-benzimidazole (1.37 g, 27.0%).

(1-3-3) Synthesis of intermediate: synthesis of 1,2-diphenyl-4-methoxy-7-(4,4,5,5-tetramethyl-1,3,2-dioxaborolan-2-yl)-1H-benzimidazole

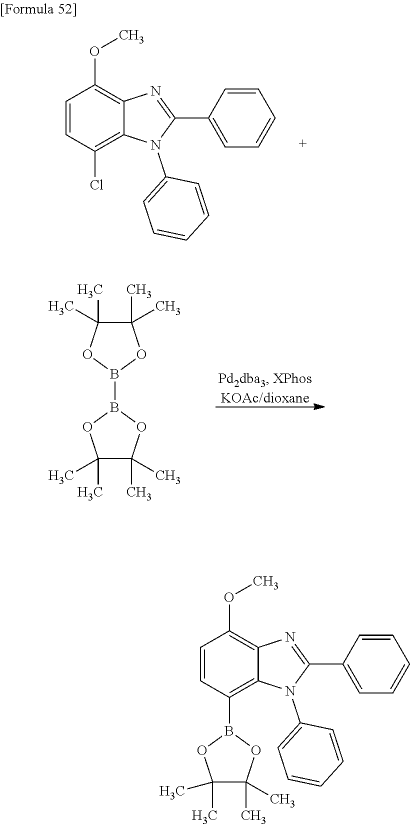

7-Chloro-4-methoxv-1,2-diphenyl-1H-benzimidazole (2.81 g, 8.4 mmol) obtained in (1-3-2), bis(pinacolato)diboron (2.32 g, 9.12 mmol), tris(dibenzylideneacetone)dipalladium (0.28 g, 0.31 mmol), XPhos (0.36 g, 0.76 mmol), and potassium acetate (2.45 g, 25 mol) were added to 29.7 mL of dioxane, and the mixture was degassed and stirred at 80° C. for 16 hours. After completion of the reaction, the mixture was concentrated under reduced pressure, water was added thereto, and the mixture was subjected to extraction with dichloromethane. The organic layer was dried over magnesium sulfate and concentrated under reduced pressure. The residue was purified by column chromatography (C300, heptane:dichloromethane) to give 1,2-diphenyl-4-methoxy-7-(4,4,5,5-tetramethyl-1,3,2-dioxaborolan-2-yl)-1H-benzimidazole (2.65 g, 74.1%).

(1-3-4) Synthesis of intermediate: synthesis of 1,2-diphenyl-4-methozy-7-(1, 10-phenanthrolin-2-y1)-1H-benzimidazole

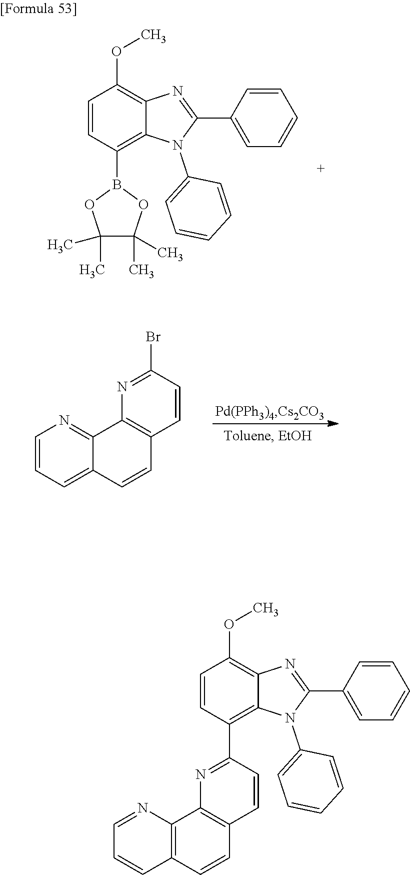

1,2-Diphenyl-4-methoxy-7-(4,4,5,5-tetramethyl-1,3,2-dioxaborolan-2-yl)-1H-benzimidazole (1.27 g, 2.98 mmol) given by conducting (1-1-3) several times, 2-bromo-1, 10-phenanthroline (0.77 is, 2.98 mmol), tetrakis(triphenylphosphine)paliadium (0.17 g, 0.15 mmol), and cesium carbonate (3.01 g, 9.24 mmol) were added to 16 mL of toluene, 3.0 mL of water, and 1.9 mL of ethanol, and the mixture was stirred at 100° C. for 16 hours. After completion of the reaction, the water was added thereto, and the mixture was subjected to extraction with dichloromethane. The organic layer was dried over magnesium sulfate and then concentrated under reduced pressure. The concentrate was purified by column chromatography (NH₂, heptane:dichloromethane) to give 1,2-diphenyl-4-methoxy-7-(1, 10-phenanthrolin-2-yl)-1H-benzimidazole (0.64 g, 45%).

(1-3-5) Synthesis of ligand: synthesis of 1,2-diphenyl-4-hydroxy-7-(1, 10-phenanthrolin-2-yl)-1H-benzimidazole

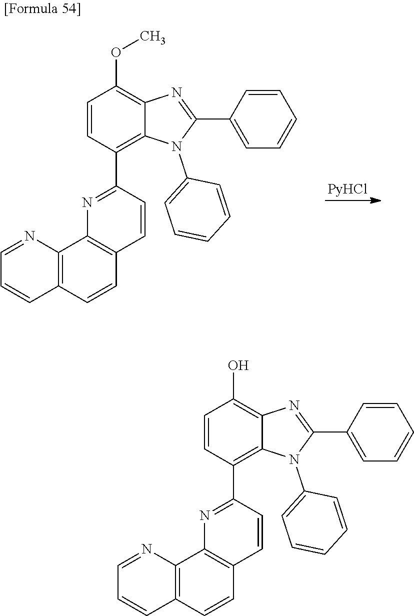 [SMILES: COc1ccc(-c2ccc3ccc4cccnc4c3n2)c2c1nc(-c1ccccc1)n2-c1ccccc1 ; "PyHCl" ; Oc1ccc(-c2ccc3ccc4cccnc4c3n2)c2c1nc(-c1ccccc1)n2-c1ccccc1]

To 1,2-diphenyl-4-methoxy-7-(1,10-phenanthrolin-2-y1)-1H-benzimidazole (0.49 g, 1.0 mmol) obtained in (1-3-4), pyridine hydrochloride (2.77 g, 24.0 mmol) was added, and the mixture was stirred at 200° C. for 16 hours. After completion of the reaction, water was added thereto, and the insoluble was filtered off to give 1,2-diphenyl-4-hydroxy-7-(1,10-phenanthrolin-2-yl)-1H-benzimidazole (L103) (0.38 g, 82.6%).

### [A-3-2] Synthesis of L103-Cs

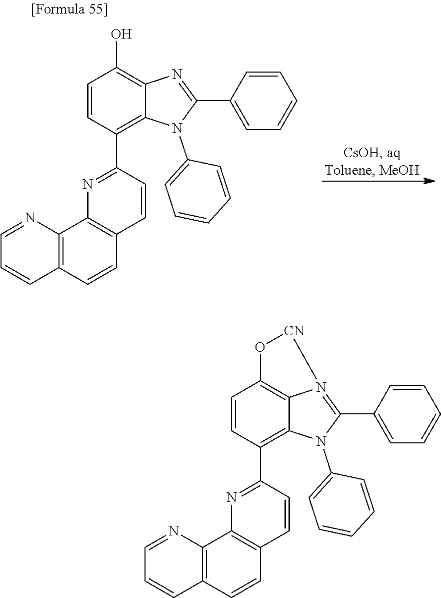

To a L103 (0.075 g, 0.161 mmol) obtained in [A-3-1]—toluene (2.15 ml) suspension, a 50% cesium hydroxide aqueous solution (0.026 mL, 0.161 mmol)—methanol (0.86 mL) solution was added dropwise, and the mixture was stirred at room temperature for one hour. After completion of the reaction, the mixture was concentrated under reduced pressure, heptane was added thereto, and then the mixture was dried. to solid to give L103-Cs (0.078 g, 81.2%).

### [A-3-3] Synthesis of L103-Li

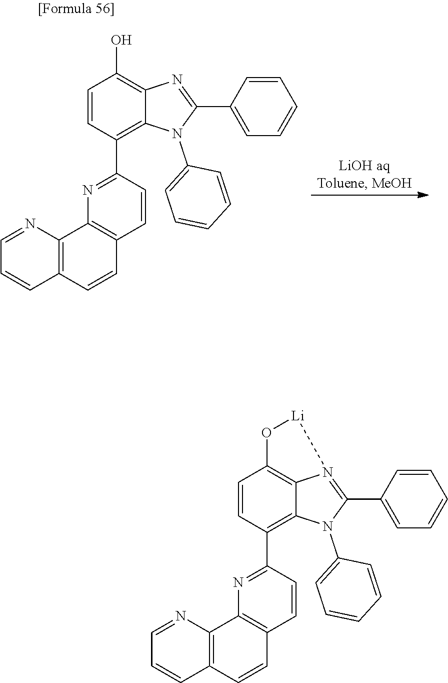

L103-Li (0.063 g, 82.9%) was given by the same operation as in [A-3-2] except that the 50% cesium hydroxide aqueous solution was replaced by a 4 mo1/L lithium hydroxide aqueous solution (0.040 mL, 0.160 mmol).

[A-3-4] Synthesis of composition containing L103-Cs (2a)

A dried solid (0.070 g, 76.2%) was given by the same operation as in [A-3-2] except that the amount of the 50% cesium hydroxide aqueous solution from 0.028 mL (0.161 mmol) to 0.022 mL (0.128 mmol) such that L103 would be in excess relative to the metal ion source (50% cesium hydroxide aqueous solution). The dried solid given was taken as a composition containing L103-Cs (2a).

[A-3-5] Synthesis of composition containing L103-Cs (2b)

A dried solid (0.105 g, 72.8%) was given by the same operation as in [A-3-2] except that the amount of the 50% cesium hydroxide aqueous solution was changed from 0.028 mL (0.161 mmol) to 0.056 mL (0.322 mmol) such that the metal ion source (50% cesium hydroxide aqueous solution) would be in excess relative to L103. The dried solid given was taken as a composition containing L103-Cs (2b).

### [C] Metal Complex Represented by Formula (3)

[C-1] Synthesis of L301-M complex (M=Cs)

### [C-1-1] Synthesis of Ligand L301

(3-1-1) Synthesis of intermediate: synthesis of 3-amino-5-chloro-2-(2,6-dimethoxyphenyl)pyridine

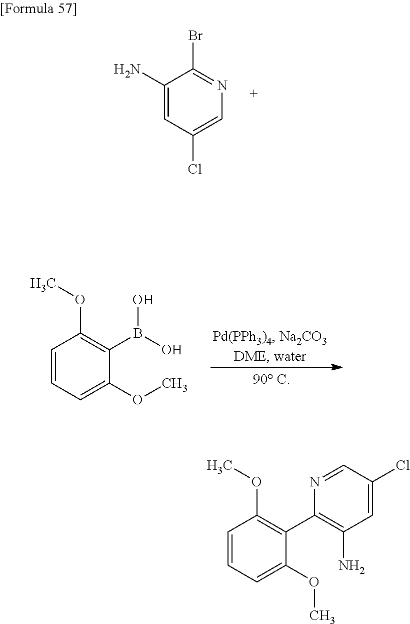 [SMILES: Nc1cc(Cl)cnc1Br ; COc1cccc(OC)c1B(O)O ; "Pd(PPh3)4, Na2CO3DME, water" ; COc1cccc(OC)c1-c1ncc(Cl)cc1N]

3-Amino-2-bromo-5-chloropyridine (4.52 g, 21.8 mmol), 2,6-dimethoxyphenylboronic acid (4.77 g, 26.2 mmol), sodium carbonate (4.62 g, 43.6 mmol), and tetrakis(triphenylphosphine)palladium (1.26 g, 10.9 mmol) were added to 80 of 1,2-dimethoxyethane and 40 mL of water, and the mixture was stirred at 90° C. for 3 hours. After completion of the reaction, the water was added thereto, and the mixture was subjected to extraction with dichloromethane. The organic layer was dried over magnesium sulfate and then concentrated under reduced pressure. The residue given was purified by column chromatography (C300, dichloromethane) to give 3-amino-5-chloro-2-(2,6-dimethoxyphenyl)pyridine (5.31 g, 92%).

(3-1-2) Synthesis of intermediate: synthesis of 3-chloro-9-methoxybenzofuro[3,2-b]pyridine

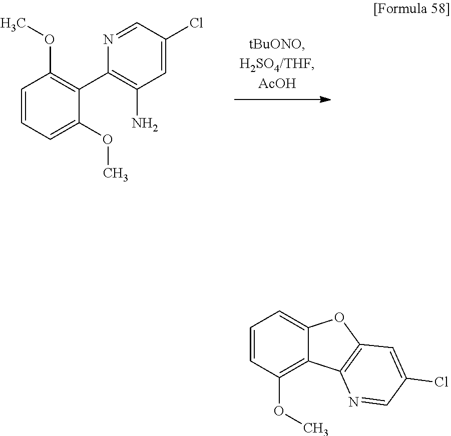 [SMILES: COc1cccc(OC)c1-c1ncc(Cl)cc1N ; COc1cccc2oc3cc(Cl)cnc3c12]

3-Amino-5-chloro-2-(2,6-dimethoxyphenyl)pyridne (4.5 g, 17 mmol) obtained in (3-1-1), sulfuric acid (0.9 mL, 16.9 mmol), and 30.2 of THF were placed in a flask and cooled to −10° C. To this solution, tert-butyl nitrite (3.6 mL, 30 mmmol) was added dropwise, and the mixture was stirred for 3 hours then stirred at room temperature for 16 hours. After completion of the reaction, the reaction solution was concentrated. Water was added thereto, the mixture was subjected to extraction with ethyl acetate, and the extract was washed with a saturated sodium hydrogen carbonate aqueous solution and saturated saline. The organic layer was dried over magnesium sulfate and then concentrated under reduced pressure. The residue was purified by column chromatography (C300, heptane:dichloromethane) to give 3-chloro-9-methoxybenzofuro[3,2-b]pyridine (2.77 g, 70%).

(3-1-3) Synthesis of intermediate: synthesis of 3-(4,4,5,5-tetramethyl-1,3,2-dioxaborolan-2-yl)-9-methoxybenzofuro[3,2-b]pyridine

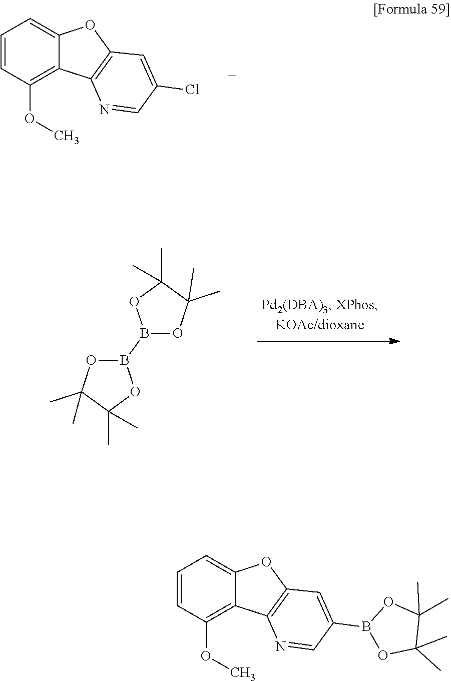

3-Chloro-9-methoxybenzofdro[3,2-b]pyridine (2.38 g, 10.2 mmol) obtained in (3-1-2), bis(pinacolato)diboron (2.88 g, 11.3 mmol),

tris(dibenzylideneacetone)dipalladium (0.35 g, 0.38 mmol), XPhos (0.443 g, 0.93 mmol), and potassium acetate (3 g, 30.6 mmol) were added to 38.4 mL of dioxane, and the mixture was stirred at 80° C. for 16 hours. After completion of the reaction, the water was added thereto, and the mixture was subjected to extraction with dichloromethane. The organic layer was dried over magnesium sulfate and then concentrated under reduced pressure. The residue was purified by column chromatography (C300, heptane:dichloromethane) to give 3-(4,4,5,5-tetramethyl-1,3,2-dioxaboralen-2-yl)-9-methoxybenzofuro[3,2-b]pyridine (2.88 g, 87.5%).

(3-1-4) Synthesis of intermediate: 3-(1,10-phenanthrolin-2-yl)-9-methoxybenzofuro[3,2-b]pyridine

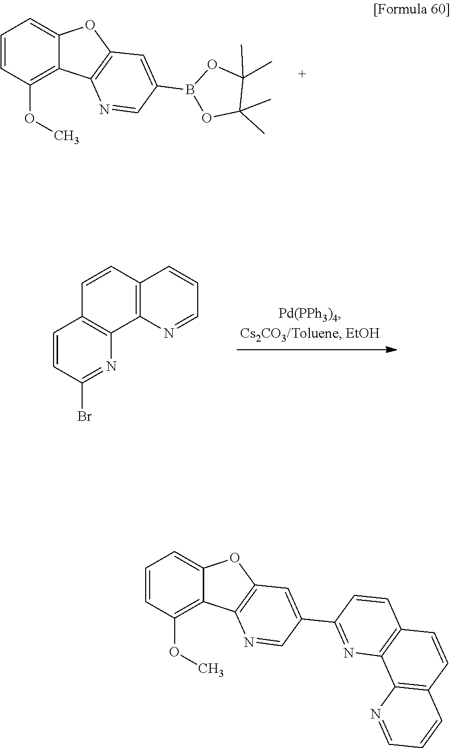 [SMILES: COc1cccc2oc3cc(B4OC(C)(C)C(C)(C)O4)cnc3c12 ; Brc1ccc2ccc3cccnc3c2n1 ; COc1cccc2oc3cc(-c4ccc5ccc6cccnc6c5n4)cnc3c12]

To 3-(4,4,5,5-tetramethyl-1,3,2-dioxaboralen-2-yl)-9-methozybenzofuro[3,2-b]pyridine (2.80 g, 8.61 mmol) obtained in (3-1-3), 2-bromo-1,10-phenanthroline (2.33 g, 9 mmol), tetrakis(triphenylphosphine)palladium (0.52 g, 0.45 mmol), and cesium carbonate (9.09 g, 27.9 mmol), 54 mL of toluene, 9 mL of water, and 5.6 mL of ethanol were added, and the mixture was stirred at 100° C. for 16 hours. After completion of the reaction, the water was added thereto, and the mixture was subjected to extraction with dichloromethane. The organic layer was dried over magnesium sulfate and then concentrated under reduced pressure. The residue was purified by column chromatography (NH₂, heptane:dichloromethane) to give 3-(1,10-phenanthrolin-2-yl)-9-methoxybenzofuro[3,2-b]pyridine (1.92 g, 58.4%).

(3-1-5) Synthesis of ligand: synthesis of 3-(1,10-phenanthrolin-2-yl)-benzofuro[3,2-b]pyridine-9-ol

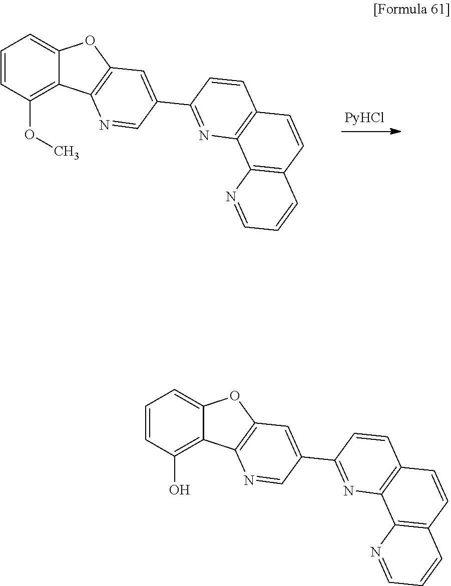 [SMILES: COc1cccc2oc3cc(-c4ccc5ccc6cccnc6c5n4)cnc3c12 ; Oc1cccc2oc3cc(-c4ccc5ccc6cccnc6c5n4)cnc3c12]

To 3-(1,10-phenanthrolin-2-yl)-9-methoxybenzofuro[3,2-b]pyridine (1.81 g, 4.8 mmol) obtained in (3-1-4), pyridine hydrochloride (12.9 g, 112 mmol) was added, and the mixture was stirred at 200° C. for 16 hours. After completion of the reaction, water was added thereto, and the precipitate was filtered off to give 3-(1,10-phenanthrolin-2-yl)-benzofuro[3,2-b]pyridine-9-ol (L301) (1.15 g, 65.8%).

[C-1-2] Synthesis of complex: Synthesis of L301-Cs complex

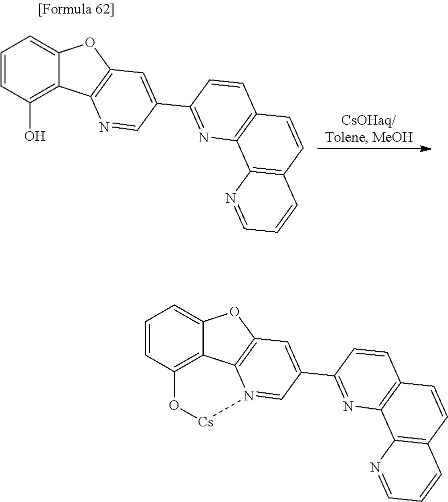

To a Ligand L301 (0.5 g, 1.38 mmol) obtained in [C-1-1]—toluene (17.6 mL) suspension, a 50% cesium hydroxide aqueous solution (0.24 mL, 1.38 mmol)—methanol (7 mL) solution was added dropwise, and the mixture was stirred at room temperature for 16 hours. The reaction solution was concentrated under reduced pressure, heptane was added thereto, and then the mixture was dried to solid to give L301-Cs (0.43 g, 63.2%).

FAB−MS: m/z=496 ([M+1]⁺)

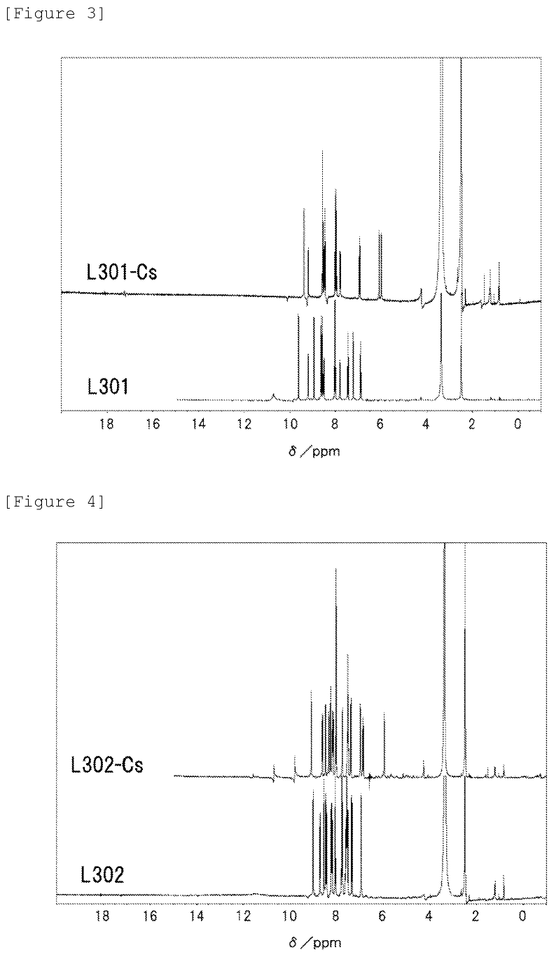

The NMR of the complex obtained is also shown in FIG. 3, together with the NMR of L301.

[C-2] Synthesis of L302-M complex (M=Cs, Rb, Li)

[C-2-1] Synthesis of Ligand L302

(3-2-1) Synthesis of intermediate: synthesis of 3-amino-2-(2,6-dimethoxyphenyl)pyridine

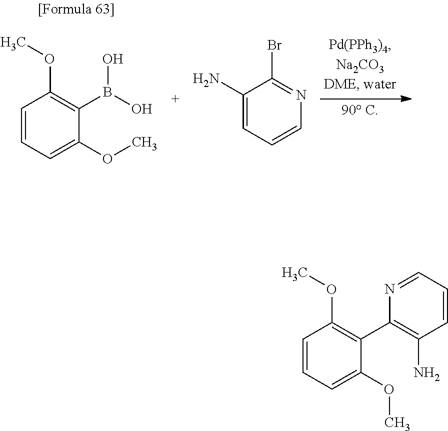 [SMILES: COc1cccc(OC)c1B(O)O ; Nc1cccnc1Br ; COc1cccc(OC)c1-c1ncccc1N]

To 3-amino-2-bromopyridine (5 g, 28.9 mmol), 2,6-dimethozyphenylboronic acid (6.26 g, 34.4 mmol), sodium carbonate (6.07 g, 57.3 mmol), and tetrakis(triphenylphosphine)palladium (1.65 g, 1.43 mmol), 105 mL of 1,2-dimethoxyethane and 52.5 ml of water were added, and the mixture was degassed and stirred at 90° C. for 3 hours. After completion of the reaction, the water was added thereto, and the mixture was subjected to extraction with dichloromethane. After concentration, the concentrate was recrystallized with heptane and dichloromethane to give 3-amino-2-(2,6-dimethoxyphenyl)pyridine (6.25 g, 93%).

(3-2-2) Synthesis of intermediate: synthesis of 9-methoxybenzofuro[3,2-b]byridine

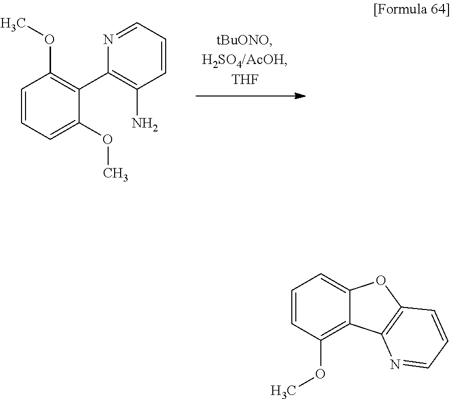 [SMILES: COc1cccc(OC)c1-c1ncccc1N ; COc1cccc2oc3cccnc3c12]

3-Amino-2-(2,6-dimethozyphenyl)pyridine (5.88 g, 25.5 mmol) obtained in (3-2-1) and sulfuric acid (1.34 mL, 25.5 mmol) were added to 46 mL of THF and 138 mL of acetic acid, and the mixture was stirred at −15° C. tert-Butyl nitrite (5.4 mL, 45 mmol) was further added thereto. The mixture was stirred at −15° C. for 3 hours and further at room temperature for 16 hours. After completion of the reaction, the mixture was concentrated under reduced pressure, and the concentrate was poured into water. Subsequently, the mixture was subjected to extraction with ethyl acetate. The organic layer was washed with a sodium hydrogen carbonate aqueous solution and saline, dried over magnesium sulfate, and concentrated under reduced pressure. The residue was purified by column chromatography (NH₂, heptane:dichloromethane), and the purified product was recrystallized with heptane to give 9-methoxybenzofuro [3,2-b]pyridine (3.7 g, 73.8%).

(3-2-3) Synthesis of intermediate: synthesis of 6-bromo-9-methozybenzofuro[3,2-b]pyridine

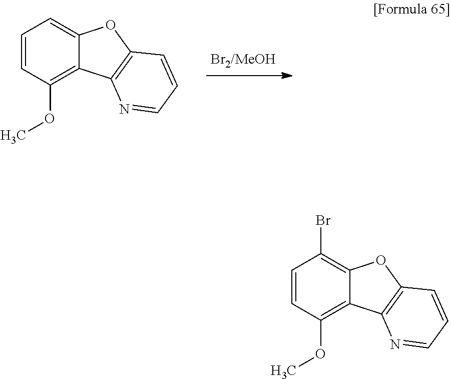 [SMILES: COc1cccc2oc3cccnc3c12 ; COc1ccc(Br)c2oc3cccnc3c12]

9-Methoxybenzofuro[3,2-b]pyridine (3.75 g, 18.8 mmol) given by conducting (3-2-2) several times was dissolved in 145 mL of methanol. The solution was cooled to 0° C., bromine (3.24 q, 20.3 mmol) was added thereto, and the mixture was stirred at room temperature for 2 hours. After completion of the reaction, the mixture was concentrated under reduced pressure, and the concentrate was poured into a 5 wt % sodium thiosulfate −5 wt % sodium hydrogen carbonate aqueous solution. Subsequently, the mixture was subjected to extraction with ethyl acetate, and the organic layer was dried over magnesium sulfate and concentrated under reduced pressure. The residue was recrystallized from heptane-ethanol to give 6-bromo-9-methoxybenzofuro[3,2-b]pyridine (9.63 g, 65.3%).

(3-2-4) Synthesis of intermediate: 9-methoxy-6-phenylphosphinoylbenzofuro[3,2-b]pyridine

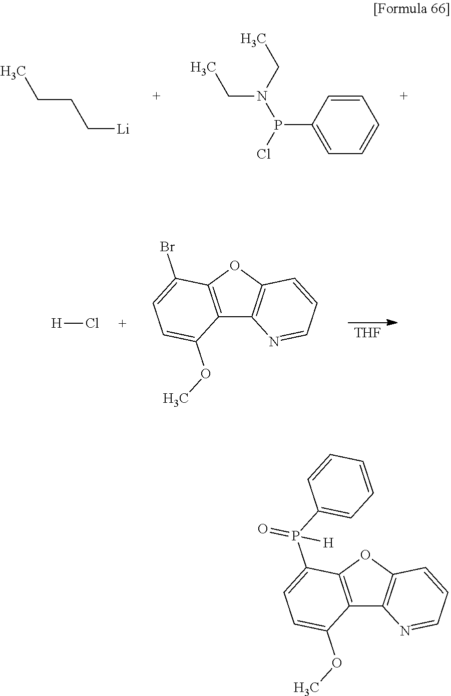

A solution of 6-bromo-9-methoxybenzofuro[3,2-b]pyridine (5.01 g, 18 mmol) obtained in (3-2-3) in 180 mL of THF was cooled to −80° C. 2.6 M n-Butyllithium (9.69 mL, 25.2 mmol) was added dropwise, and the mixture was stirred for 2 hours.

Diethylamino(chloro)phenylphosphine (5.43 g, 25.2 moml) was further added thereto, and the mixture was stirred for 30 minutes and further at room temperature.

Thereafter, the mixture was cooled to 80°C., hydrochloric acid was added thereto, and the mixture was stirred at room temperature. After completion of the reaction, the mixture was concentrated under reduced pressure, and the concentrate was neutralized with a sodium hydrogen carbonate aqueous solution. Subsequently, the mixture was subjected to extraction with dichloromethane. The organic layer was dried over magnesium sulfate and then concentrated under reduced pressure. The residue was purified by column chromatography to give 9-methoxy-6-phenylphosphinoylbenzofuro[3,2-b]pyrdine (4.25 g, 72.8%).

(3-2-5) Synthesis of intermediate: synthesis of 9-methoxy-6-(phenanthrolyl(phenyl)phosphinoyl)benzofuro[3, 2-b]pyridine

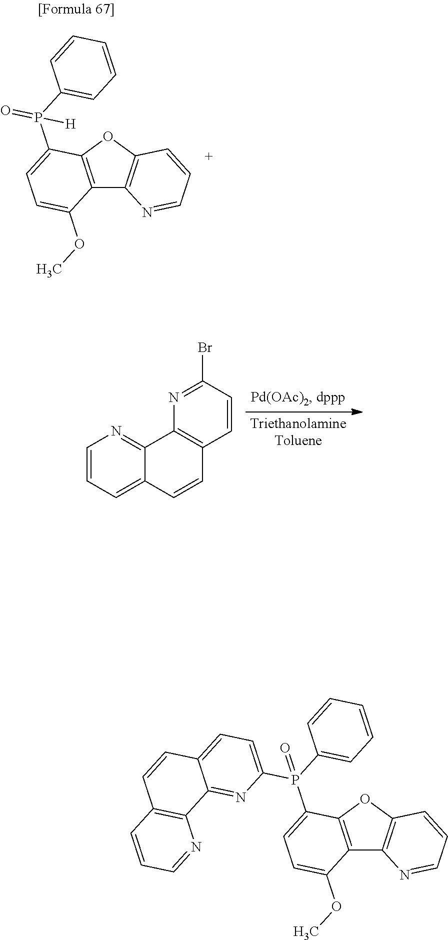 [SMILES: COc1ccc([PH](=O)c2ccccc2)c2oc3cccnc3c12 ; Brc1ccc2ccc3cccnc3c2n1 ; COc1ccc(P(=O)(c2ccccc2)c2ccc3ccc4cccnc4c3n2)c2oc3cccnc3c12]

9-Methoxy-6-phenylphosphinoylbenzofuro[3,2-b]pyridine (3.01 g, 12 mmol) obtained in (3-2-4), 2-bromophenanthroline (3.73 g, 14.4 mmol), palladium acetate (54 mg, 0.24 mmol), 1,3-bis(diphenylphosphino)propane (198 mg, 0.48 mmol), and 24 mL of triethanolamine were added to 48 mL of toluene, and the mixture was stirred at 140° C. for 30 hours. After completion of the reaction, the mixture was concentrated under reduced pressure, the residue given was poured into water, and the mixture was subjected to extraction with dichloromethane. The organic layer was washed with water, then dried over magnesium sulfate, and concentrated under reduced pressure. The residue was purified by column chromatography (NH₂, heptane:dichloromethane) to give 9-methoxy-6-(phenanthrolyl(phenyl)phosphinyl)benzofuro[3,2-b]pyridine (3.01 g, 50%).

(3-2-6) Synthesis of ligand: synthesis of 9-hydroxy-6-(phenanthrolyl(phenyl)phosphinoyl)benzofuro[3,2-b]pyridine (L302)

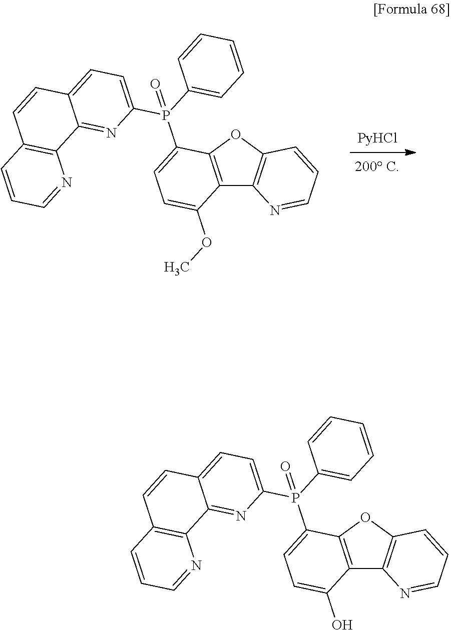 [SMILES: COc1ccc(P(=O)(c2ccccc2)c2ccc3ccc4cccnc4c3n2)c2oc3cccnc3c12 ; O=P(c1ccccc1)(c1ccc2ccc3cccnc3c2n1)c1ccc(O)c2c1oc1cccnc12]

9-Methoxy-6-phenanthrolyl(phenyl)phosphinoylbenzofuro[3,2-b]pyridine (1.50 g, 3.0 mmol) obtained in (3-2-5) was added to 8.12 g of pyridine hydrochloride, and the mixture was stirred at 200° C. for one hour. After completion of the reaction, the water was added thereto, and the insoluble was filtered off. The insoluble given was dissolved in dichloromethane. The solution was washed with a saturated NaHCO₃ aqueous solution, a saturated ammonium chloride aqueous solution, and water in the order mentioned, dried over magnesium sulfate, and then concentrated under reduced pressure. The residue was recrystallized from heptane-ethanol to give 9-hydroxy-6-(phenanthrolyl(phenyl)phosphinoyl)benzofuro[3,2-b]pyridine (L302) (904 mg, 61.7%).

[C-2-2] Synthesis of complex: Synthesis of L302-Cs complex

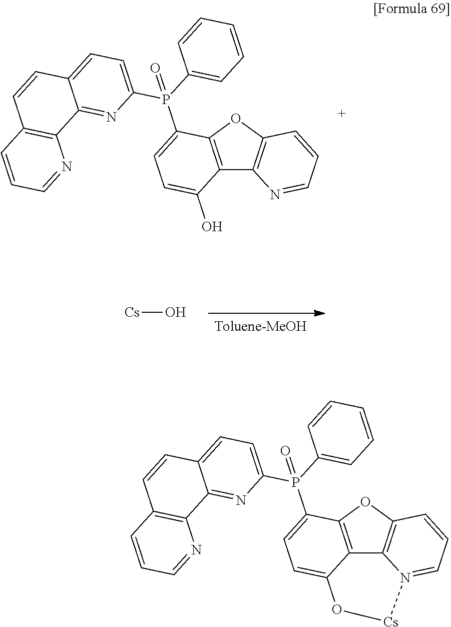

To a Ligand L302 given by conducting [C-2-1] several times (0.98 g, 0.2 mmol)-toluene (2.5 mL) solution, a 50% cesium hydroxide aqueous solution (0.035 mL, 0.2 mmol)-methanol (1 mL) solution was added dropwise, and the mixture was stirred at room temperature for one hour. The reaction solution was concentrated under reduced pressure, heptane was added thereto, and then the mixture was dried to solid to give L302-Cs (71 mg, 57.5%).

FAB−MS: m/z=620 ([M+1]⁺)

The NMR of the complex obtained is also shown in FIG. 4, together with the NMR. of L302.

[C-2-3] Synthesis of complex: Synthesis of L302-Rb complex

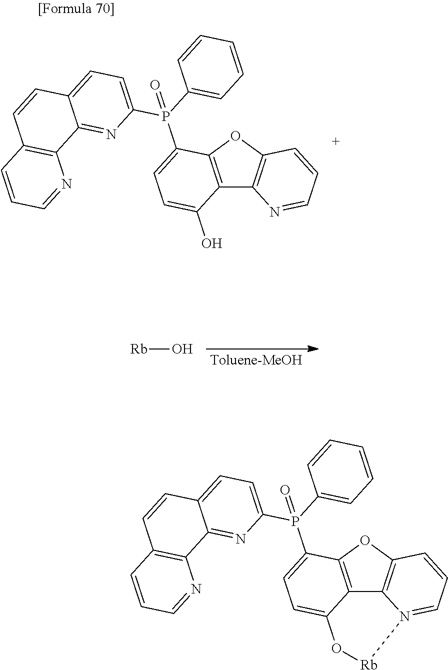

To a Ligand L302 given by conducting [C-2-1] several times (0.98 g, 0.2 mmol)-toluene (2.5 mL) solution, a 50% rubidium hydroxide aqueous solution (0.041 mL, 0.2 mmol)-methanol (1 mL) solution was added dropwise, and the mixture was stirred at room temperature for one hour. The reaction solution was concentrated under reduced pressure, heptane was added thereto, and then the mixture was dried to solid to give L302-Rb (87 mg, 70.5%).

FAB−MS: m/z=573 ([M+1]⁺)

[C-2-4] Synthesis of complex: Synthesis of L302-Li complex

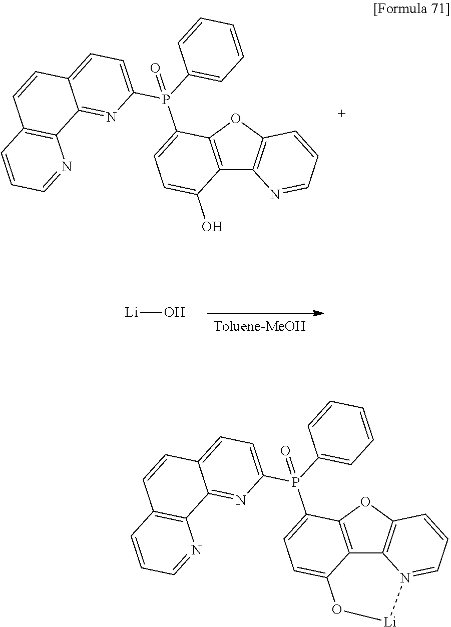

To a Ligand L302 (0.146 g, 0.3 mmol) obtained in [C-2-1]—toluene (3.75 mL) solution, a 4 mol/L lithium hydroxide aqueous solution (0.075 mL, 0.3 mmol)—methanol (1.5 mL) solution was added dropwise, and the mixture was stirred at room temperature for one hour. The reaction solution was concentrated under reduced pressure, heptane was added thereto, and then the mixture was dried to solid to give L302-Li (136 mg, 92.0%).

[C-3] Synthesis of L303-14 complex (M=Cs, Rb)

[C-3-1] Synthesis of Ligand L303

(3-3-1) Synthesis of intermediate: synthesis of 6-(4,4,5,5-tetramethyl-1,3,2-dioxaborolan-2-yl)9-methoxybenzofuro[3,2-b]pyridine

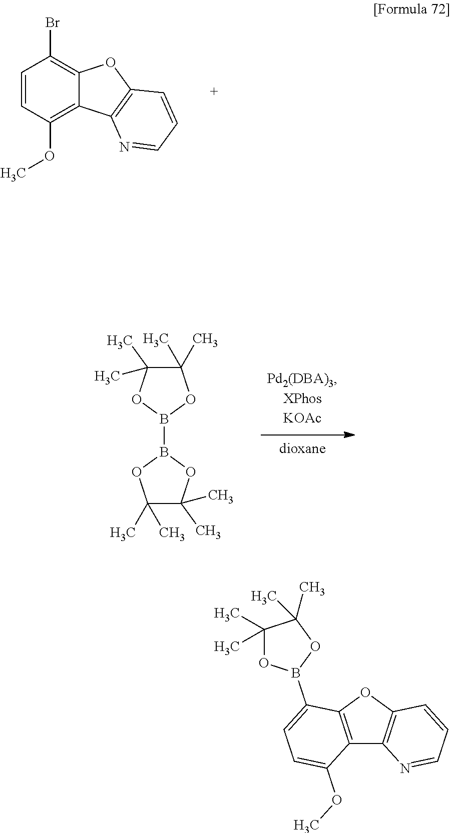

6-Bromo-9-methoxybenzofuro[3,2-b]pyridine (5.0 g, 18 mmol) obtained in the same manner as in (3-2-3) of [C-2], bis(pinacolato)diboron (7.26 g, 28.6 mmol), tris(dibenzylideneacetone)dipalladium (0.577 g, 0.63 mmol), XPhos (0.377 g, 0.79 mmol), and potassium acetate (5.57 g, 56.8 mmol) were added to 66 mL of dioxane, and the mixture was degassed and stirred at 80° C. for 16 hours. After completion of the reaction, the mixture was concentrated under reduced pressure, and the concentrate was subjected to extraction with dichloromethane. The organic layer was dried over magnesium sulfate and then concentrated under reduced pressure. The residue was purified by column chromatography (C300, heptane:dichloromethane). The purified product was recrystallized with methanol to give 6-(4,4,5,5-tetramethyl-1,3,2-dioxaborolan-2-yl)9-methoxybenzofuro[3,2-b]pyridine (3.11 g, 53%).

(3-3-2) Synthesis of intermediate: synthesis of 6-(9-chloro-1,10-phenanthrolin-2-yl)-9-methoxybenzofuro[3,2-b]pyridine

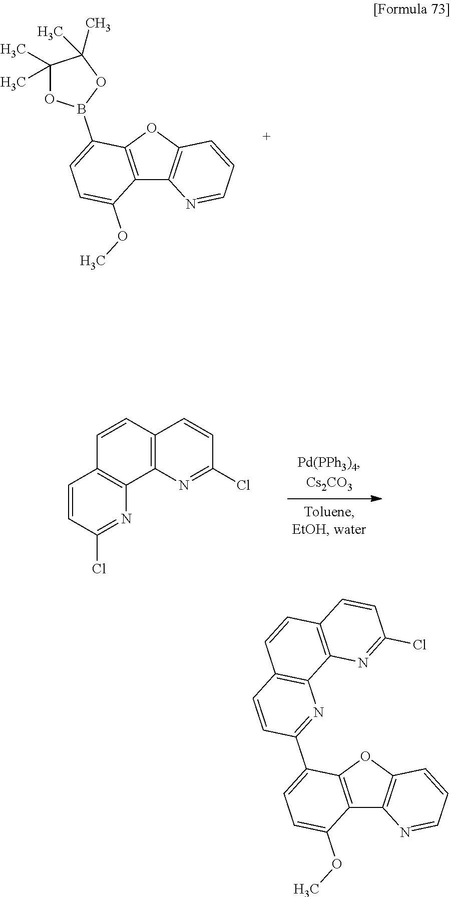 [SMILES: COc1ccc(B2OC(C)(C)C(C)(C)O2)c2oc3cccnc3c12 ; Clc1ccc2ccc3ccc(Cl)nc3c2n1 ; COc1ccc(-c2ccc3ccc4ccc(Cl)nc4c3n2)c2oc3cccnc3c12]

6-(4,4,5,5-tetramethyl-1,3,2-dioxaborolan-2-yl)9-methoxybenzofuro[3,2-b]pyridine given by conducting (3-3-1) several times (3.2 g, 9.84 mmol), 2,9-dichloro-1,10-phenanthroline (2.46 g, 9.82 mmol), tetrakis(tripenylphosphine)palladium (0.382 g, 0.504 mmol), and cesium carbonate (10.26 g, 31.6 mmol) were added to toluene (60.8 mL), ethanol (5.6 mL), and water (8.96 mL), and the mixture was degassed and stirred at 100° C. for 16 hours. After completion of the reaction, the mixture was concentrated under reduced pressure, water was added thereto, and the mixture was subjected to extraction with dichloromethane. The organic layer was dried over magnesium sulfate and then concentrated under reduced pressure. The residue was recrystallized with heptane-dichloromethane to give 6-(9-chloro-1,10-phenanthrolin-2-yl)-9-methoxybenzofuro[3,2-b]pyridine (1.43 g, 71.0%).

(3-3-3) Synthesis of intermediate: synthesis of 6-(9-diphenylphosphinoyl-1,10-phenanthrolin-2-yl)-9-methoxybenzofuro[3,2-b]pyridine

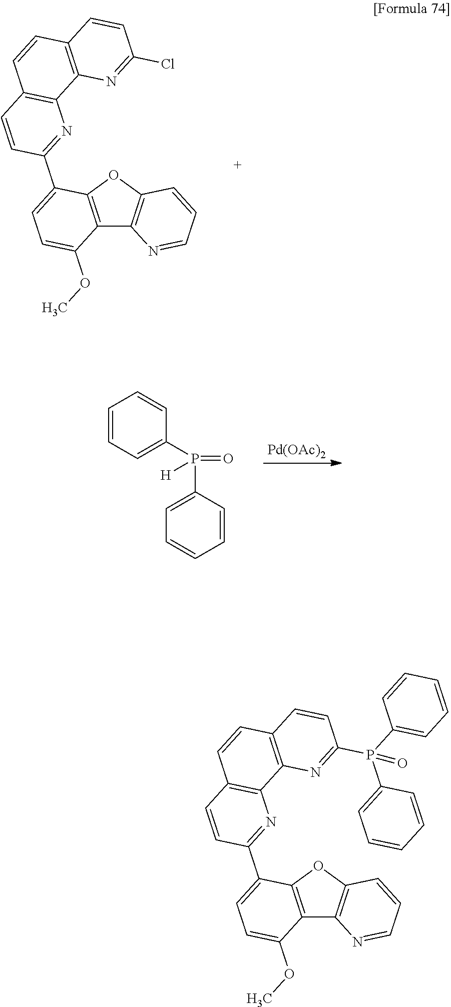 [SMILES: COc1ccc(-c2ccc3ccc4ccc(Cl)nc4c3n2)c2oc3cccnc3c12 ; O=[PH](c1ccccc1)c1ccccc1 ; COc1ccc(-c2ccc3ccc4ccc(P(=O)(c5ccccc5)c5ccccc5)nc4c3n2)c2oc3cccnc3c12 ; "Pd(OAc)2"]

Synthesis was conducted with reference to Patent Literature: Japanese Patent Laid-Open No. 2017-120845 to give 6-(9-diphenylphosphino-1, 10-phenanthrolin-2-yl)-9-methoxybenzofuro[3,2-b]pyridine (1.7 g, 60.7%).

(3-3-4) Synthesis of Ligand L103: synthesis of 9-hydroxy-6-(9-diphenylphosphino-1,10-phenanthrolin-2-yl)-benzofuro[3,2-b]pyridine

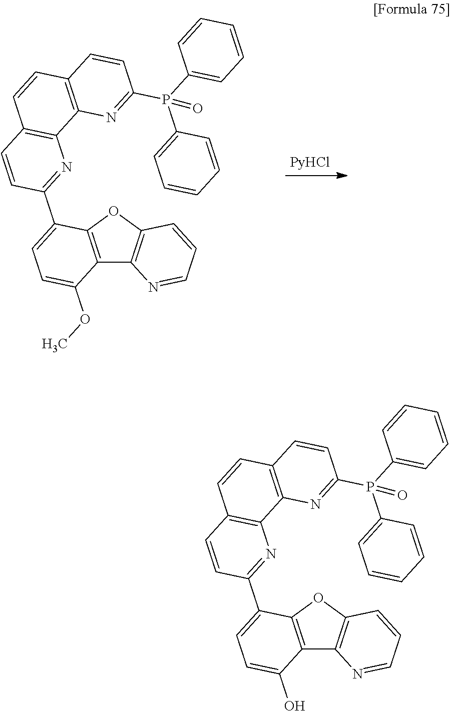 [SMILES: COc1ccc(-c2ccc3ccc4ccc(P(=O)(c5ccccc5)c5ccccc5)nc4c3n2)c2oc3cccnc3c12 ; "PyHCl" ; O=P(c1ccccc1)(c1ccccc1)c1ccc2ccc3ccc(-c4ccc(O)c5c4oc4cccnc45)nc3c2n1]

To 6-(9-diphenylphosphino-1,10-phenanthrolin-2-yl)-9-methozybenzofuro[3,2-b]pyridine (0.46 g, 0.8 mmol) obtained in (3-3-3), pyridine hydrochloride (2.4 g, 21 mmol) was added, and the mixture was stirred at 200° C. for 16 hours. After completion of the reaction, water was added thereto, and the precipitate formed was filtered off. The precipitate was further washed with methanol to give 9-hydroxy-6-(9-diphenylphosphino-1,10-phenanthrolin-2-yl)-benzofuro[3,2-b]pyridine (L303) (0.30 g, 68%).

[C-3-2] Synthesis of complex: synthesis of L303-Cs

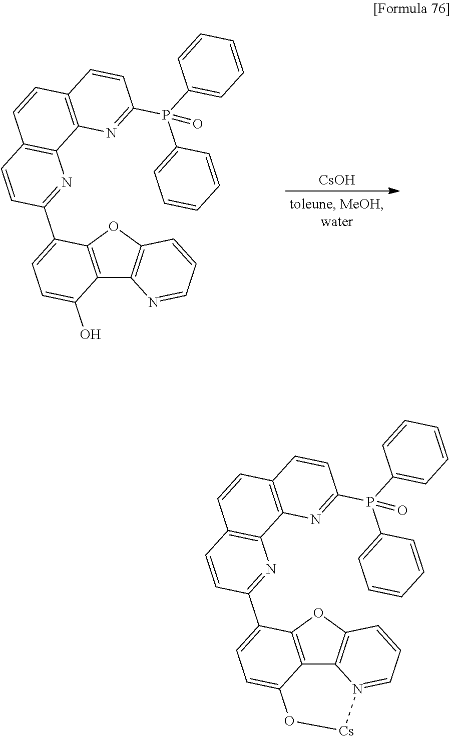

To a Ligand L303 (0.10 g, 0.177 mmol) obtained in [C-3-1]—toluene (4 mL) solution, a 50% cesium hydroxide aqueous solution (0.031 mL, 0.177 mmol)—methanol (1.5 mL) solution was added dropwise, and the mixture was stirred at room temperature for 16 hours. The reaction solution was concentrated under reduced pressure, heptane was added thereto, and then the mixture was dried to solid to give L103-Cs (0.09 g, 72%).

FAB−MS: m/z=696 ([M+1]⁺)

[C-3-3] Synthesis of complex: synthesis of L303-Rb

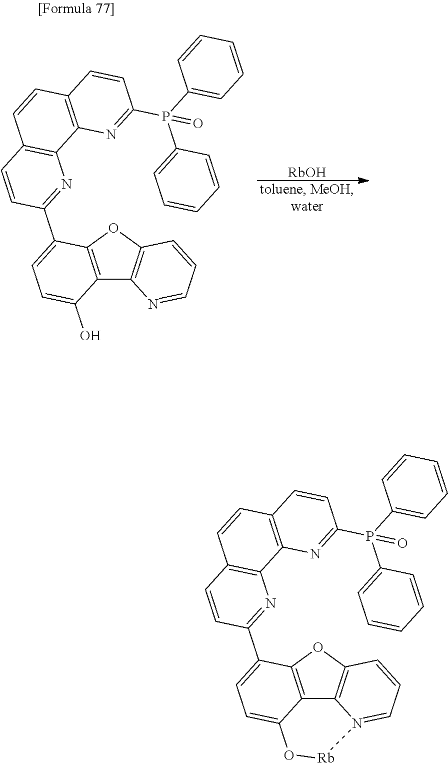

To a Ligand L303 (0.10 g, 0.177 mmol) obtained in [C-3-1]—toluene (2.7 mL) solution, a 50% rubidium hydroxide aqueous solution (0.021 mL, 0.177 mmol)—methanol (1 mL) solution was added dropwise, and the mixture was stirred at room temperature for 16 hours. The reaction solution was concentrated under reduced pressure, heptane was added thereto, and then the mixture was dried to solid to give L303-Rb (0.06 g, 56.5%).

FAB−MS: m/z=649 ([M+1]⁺)

### [2] Production and Evaluation of Organic Electroluminescent Element

### (1) PRODUCTION OF LIQUID MATERIAL

The metal complexes obtained in [1] were each dissolved in a protic polar solvent shown in Table 1 to produce a liquid material for constructing the electron transport layer of an organic electroluminescent element.

For example, Metal complex L101-Cs was dissolved in 1-heptanol to prepare alcohol solutions of 7.5 g/L to 15 g/L.

The results of dissolution test for each solvent are shown in Table 1. In Table 1, a case where an undissolved remnant was present was evaluated as “X”, a case where a slight undissolved remnant was present was evaluated as “Δ”, and a case of complete dissolution was evaluated as “ο”.

TABLE 1

| Material | Solvent | 15 g/L | 7.5 g/L | 5 g/L |
|---|---|---|---|---|
| L101-Cs | 1-Heptanol | x | ○ | — |
| L101-Rb | 1-Heptanol | x | ○ | — |
| L102-Cs | 1-Heptanol | x | ○ | — |
| Composition containing | 2-Methoxyethanol | x | ○ | — |
| L102-Cs (1a) | | | | |
| Composition containing | 1-Heptanol | ○ | ○ | — |
| L102-Cs (1b) | | | | |
| L103-Cs | 1-Heptanol | x | ○ | — |
| L103-Li | 1-Heptanol | x | ○ | — |
| Composition containing | 1-Heptanol | x | Δ | ○ |
| L103-Cs (2a) | | | | |
| Composition containing | 1-Heptanol | ○ | ○ | — |
| L103-Cs (2b) | | | | |
| L301-Cs | 1-Heptanol | x | Δ | ○ |
| L301-Cs | 2-Methoxyethanol | ○ | ○ | — |
| L302-Cs | 1-Heptanol | ○ | ○ | — |
| L302-Rb | 1-Heptanol | ○ | ○ | — |
| L302-Li | 1-Heptanol | ○ | ○ | — |
| L303-Cs | 1-Heptanol | x | ○ | — |
| LS03-Rb | 1-Heptanol | x | ○ | — |

### (2) PRODUCTION OF ORGANIC ELECTROLUMINESCENT ELEMENT

### (2-1) Element Confguration

Produced were an organic electroluminescent element of the element configuration. shown in FIG. 1 (Element (A)) and an organic electroluminescent element having an element configuration exactly same as that shown in FIG. 1 except that an electron inlection layer is provided between the cathode and the electron transport layer (element (B)). The film thickness of each layer is as follows.

Element (A): anode/hole injection layer/hole transport layer/light emitting layer/electron transport layer/ cathode

Anode: ITO (150 nm)

Hole injection layer: PEDOT: PSS (35 nm)

Hole transport layer: triphenylamine polymer (20 cm)

Light emitting layer: F8BT (manufactured by Aldrich, CAS: 210347-52-7) (60 nm)

Electron transport layer: electron transporting material shown in Table 2 (20 nm)

Cathode: AL (100 nm)

Element (B): anode/hole injection layer/hole transport layer/light emitting layer/electron transport layer/electron injection layer/cathode

Anode: ITO (150 nm)

Hole injection layer: PEDOT: PSS (35 nm)

Hole transport layer: triphenylamine polymer (20 nm)

Light emitting layer: F8BT (manufactured by Aldrich, CAS: 210347-52-7) (60 nm)

Electron transport layer: electron transporting material shown in Table 2 (20 nm)

Electron injection layer: LiF (0.5 nm)

Cathode: AL (100 nm)

### (2-2) Materials Used

Used was an ITO substrate manufactured by Techno Print Co., Ltd. (film thickness: 150 nm). As 2-propanol for use in washing of the substrate, used was 2-propanol for electronics industry manufactured by Wako Pure Chemical Industries, Ltd.

### Hole Injection Layer

As the liquid material for forming a hole injection layer, PEDOT:PSS (AI4083 manufactured by Heraeus) was used as it was without dilution.

### Hole Transport Layer

As the liquid material for forming a hole transport layer, used was a solution of triphenylamine polymer (5 g/L) in toluene including 1 phr dicumyl peroxide added. The toluene used was manufactured by Wake Pure Chemical Industries, Ltd.

The compounds used are shown below.

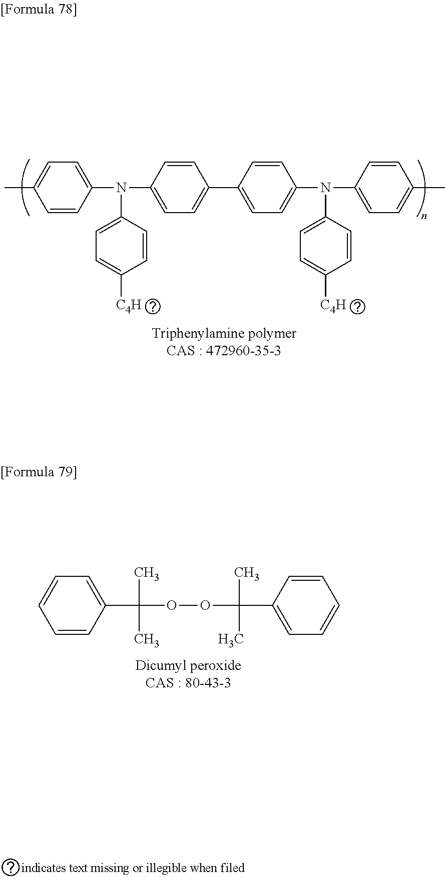

### Light Emitting Layer

For formation of the light emitting layer, a solution of F8BT (10 g/L) in toluene was used. The toluene used was manufactured by Wako Pure Chemical Industries, Ltd.

The compounds used are shown below.

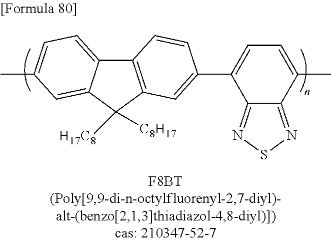

### Electron Transport Layer

For formation of the electron transport layer, used were liquid materials for forming an electron transport layer shown in Table 2. The solvents used were manufactured by Wako Pure Chemical Industries, Ltd.

Liquid materials for forming an electron transport layer each were prepared by dissolving a metal complex shown in Table 2 at a concentration of 7.5 g/L in a solvent shown in Table 2.

In order to make elements including a metal alkoxide added for the purpose of achieving a further driving voltage and a longer lifetime, also prepared were liquid materials for forming an electron transport layer including a metal alkoxide as a dopant. The liquid materials for forming an electron transport layer including a metal alkoxide added each were prepared by adding a metal alkoxide solution to the solution of a metal complex. The solution of a metal complex was prepared by dissolving a metal complex shown in Table 2 at a concentration of 7.5 g/L in a solvent shown in Table 2. The metal alkoxide solution was, in the case of lithium-n-butoxide (LiOnBu), prepared by dissolving a reagent manufactured by Kojundo hemical Lab. Co., Ltd. at a concentration of 5 g/L in a solvent shown in Table 2 in a glove box. Next, the 7.5 g/L metal complex solution and the 5 g/L metal alkoxide solution were mixed such that the content of the dopant (metal alkoxide) reached 10% by weight with respect to the metal complex, and then the mixed solution was subjected to film formation.

A comparative liquid material for forming an electron transport layer was prepared in the same manner as described above except that LiBPP was used instead of the metal complex.

The compound used is shown below.

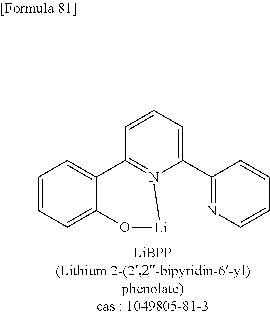

### (2-3) Production of Element

For pretreating the ITO substrate, the substrate was washed by boiling in 2-propanol for 5 minutes, immediately thereafter placed in a UV/O₃ treatment apparatus, and O₃-treated with UV irradiation for 15 minutes.

The hole injection layer and hole transport layer, light emitting layer, and electron transport layer were formed using a spin coater manufactured by IDEN and then dried under an N₂ atmosphere.

For deposition of the cathode (Al, purity: 99.999%) and the electron injection layer (LiF), employed was a high vacuum deposition apparatus having a chamber thickness of 1×10⁻⁴ Pa. The deposition rate for LiF was 0.1 Å/s, and that for Al was 5 Å/s. After film formation of the cathode completed, the element was immediately transferred into a glove box purged with nitrogen and sealed with a glass cap coated with a desiccant.

### (3) EVALUATION OF ORGANIC ELECTROLUMINESCENT ELEMENT

For the voltage-current-luminance characteristic of the organic EL element made, a voltage from 0 V to 10 V was applied, and the current value was measured every 0.1 V using a DC voltage current source/monitor (6241A and 7351A manufactured by ADCMT).

The lifetime of the organic EL element made was measured using a lifetime evaluation measuring apparatus (manufactured by Kyushu Keisokki Co., Ltd.). The element was placed in the thermostatic chamber at a constant temperature of 25° C., and the changes in the luminance voltage associated with constant current driving were measured. In this case, the acceleration factor for evaluation of the element was 1.758. Comparison was made based on the values of half-life, at which the luminance dropped to the half of the initial luminance, obtained in accordance with the driving time converted in terms of 100 cd/m².

T=(L₀/L₁)^{1.758}×T₁

wherein L₀: initial luminance [cd/m²], L: converted luminance [cd/m²], T₁: measured luminance half-life, and T: converted luminance half-life

The relative lifetime was based on the lifetime of Example 3 [material complex (L301-Cs)+dopant (LiOBu)+electron injection layer](100).

### (4) EXAMPLES AND COMPARATIVE EXAMPLES

### 1) Example 1

In (2) Production of Organic Electroluminescent Element described above, using L101-Cs as the metal complex of the electron transporting material and. LiOBu as the dopant, an element including no electron injection layer (A) or an element including an electron injection layer (B) was produced. The physical property, values of driving voltage (V), current efficiency (η_{c}), and relative lifetime of the elements given were also shown in Table 2. The presence or absence of the electron injection. layer was also described in Table 2.

### 2) Examples 2 to 7, Comparative Example 1

An element was produced in the same manner as in Example 1 except that the liquid material for forming an electron transport layer was replaced by one shown in Table 2 in Example 1. The physical property values of driving voltage (V), current efficiency (η_{c}), and relative lifetime of the elements given were also shown in Table 2.

### (5) EVALUATION AND DISCUSSION

First, it can be seen that elements each including the compound of each of the present Examples (compounds having, as the basic skeleton, a nitrogen-containing fused ring formed by fusion of a phenolate and rings including a N-containing hetero ring) (Examples 1 to 7) have a lower driving voltage, higher current efficiency, and a longer lifetime than those of the similar compound LiBPP of Comparative Example 1 (having a compound having a structure in which phenolate is linked to a pyridine ring). Although the reason for this is not clear, it is conceived that this is because the compounds of the present Examples, each of which has a structure in which a phenolate is fused with a pyridine ring or an imidazole ring, have improved film forming properties and electron transportability, relative to the compound of Comparative Example 1. LiBPP of Comparative Example 1 has a total of 3 carbocycles and heterocycles, whereas the compounds of the present Examples each have a total of 6 or more carbocycles and heterocycles. This is also conceived to contribute to improvement in the film forming properties and electron transportability.

L302-Cs used in Examples 4 and 5 has a structure of a phosphine oxide, in which the C—P bond is reported to be chemically unstabilised in an anionic state. However, it can be seen that the elements of Examples 4 and 5 have achieved a longer lifetime than that of Comparative Example 1. Although the reason for this is not clear, it is conceived that this is because, in spite of the structure having a phosphine oxide, having a complex structure as shown in the present invention has allowed the electron transportability to be improved.

Also, it can be seen from the comparison between Example 4 and Example 5 that addition of a metal alkoxide allows a lower driving voltage and a longer lifetime to be achieved.

TABLE 2

| | Liquld material for forming an electron transport layer | | | | Electron | | |
|---|---|---|---|---|---|---|---|
| | Electron transporting materlal | | | injection | Driving | Current | Relative |
| | Metal complex | Dopant | Solvent | layer | voltage | efficiency | lifetime |
| Example | L101-Cs | LiOBu | 1-Heptanol | — | 4.7 | 3.2 | 72 |
| 1 | | | | ◯ | 4.6 | 3 | 74 |
| Example | L101-Rb | LiOBu | 1-Heptanol | — | 5 | 3.5 | 74 |
| 2 | | | | ◯ | 5 | 3.4 | 73 |
| Example | L301-Cs | LiOBu | 2-Methoxyethanol | — | 4.4 | 3.4 | 90 |
| 3 | | | | ◯ | 4.3 | 3.2 | 100 |
| Example | L303-Cs | — | 1-Heptanol | — | 6.9 | 2.8 | 28 |
| 4 | | | | ◯ | 5.7 | 3.4 | 18 |
| Example | L302-Cs | LiOBu | 1-Heptanol | — | 5.3 | 3.4 | 45 |
| 5 | | | | ◯ | 4.9 | 2.6 | 80 |
| Example | L302-Li | LiOBu | 1-Heptanol | — | 6.2 | 2.1 | 11 |
| 6 | | | | ◯ | 6 | 1.9 | 11 |
| Example | L303-Rb | LiOBu | 1-Heptanol | — | 4.5 | 2.9 | 16 |
| 7 | | | | ◯ | 4.5 | 2.9 | 16 |
| Comparative | K iBPP | LiOBu | 2-Methoxyethanol | — | 7.1 | 1.5 | 8 |
| Example 1 | | | | ◯ | 6.7 | 1 | 7 |

Also with compositions containing L102-Cs (1a) and (1b) and compositions containing L103-Cs (2a) and (2b), liquid materials for forming as electron transport layer were also prepared using 2-methoxyethanol or 1-heptanol. The liquid materials were used to produce organic electroluminescent elements. Light emission from the elements produced was able to be confirmed.

### INDUSTRIAL APPLICABILITY

The metal complex having a novel ligand of the present invention can achieve both high durability and electron transportability and can be suitably used as an electron transporting material for organic electrolaminescent elements.

### REFERENCE SIGNS LIST

1 Organic electroluminescent element
2 Substrate
3 Anode
4 Hole injection layer
5 Hole transport layer
6 Light emitting layer
7 Electron transport layer
8 Cathode
9 Sealing member

1. A metal complex represented by the following Formula (1) to the following Formula (3), comprising at least one phenanthrolinyl group and a nitrogen-containing fused ring: 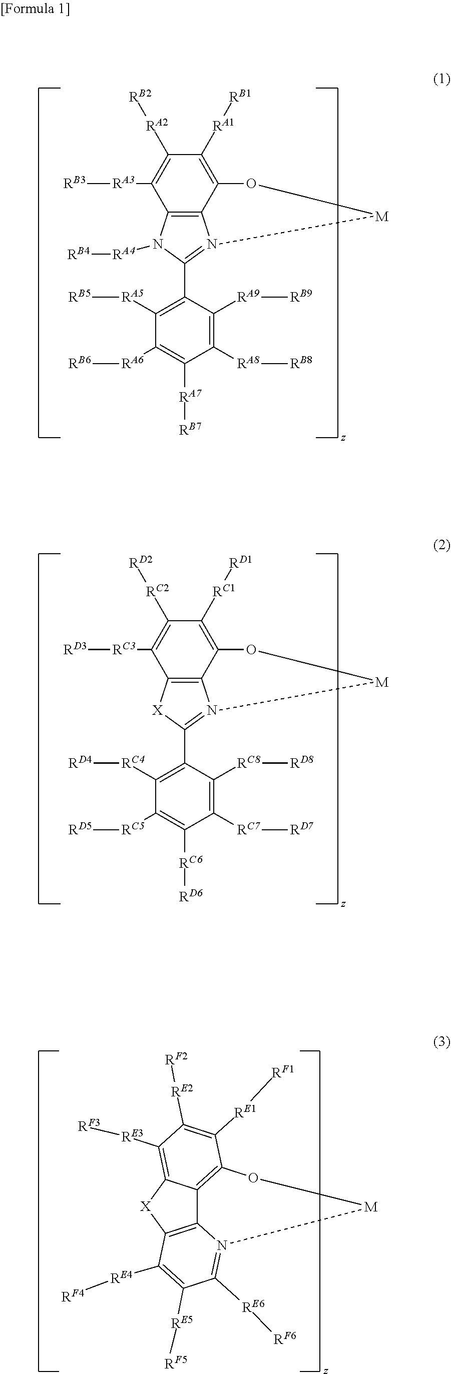
wherein, in Formula (1) to Formula (3),
R^{A1} to R^{A9}, R^{C1} to R^{C8}, and R^{E1} to R^{E6} are each independently a single bond, an alkylen group, an arylene group, a heteroarylene group, or a group represented by the following Formula (4):
[Formula 2]
—R^{P1}—P(═O)R^{P2}—R^{P3}— (4)
wherein, in Formula (4), R^{P1} and R^{P3} are each independently a single bond, an alkylene group, an arylene group, or a heteroarylene group, and R^{P2} is an alkyl group, an aryl group, or a heteroaryl group,
R^{B1} to R^{B9}, R^{D1} to R^{D8}, and R^{F1} to R^{F6} are each independently a hydrogen atom, an alkyl group, an aryl group, a heteroaryl group, an alkoxy group, an aryloxy group, a heteroaryloxy group, an amino group, a cyano group, a halogen atom, or a hydroxy group,
one or more selected from the group consisting of R^{B1} to R^{B9} are a phenanthrolinyl group, one or more selected from the group consisting of R^{D1} to R^{D8} are a phenanthrolinyl group, and one or more selected from the group consisting of R^{F1} to R^{F6} are a phenanthrolinyl group,
M is an alkali metal or an alkaline earth metal,
Z is 1 or 2, and
X is O or S.

2. The metal complex according to claim 1, wherein the phenanthrolinyl group is selected from the group consisting of the following Formula (5a) to the following Formula (5d): 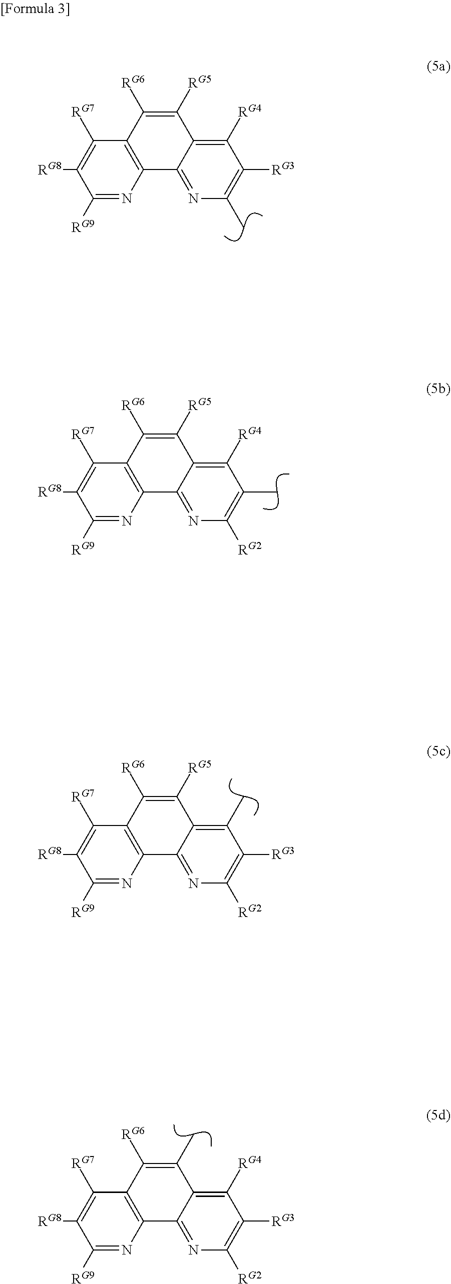
wherein, in Formula (5a) to Formula (5d), R^{G2} to R^{G9} are each independently a hydrogen atom, an alkyl group, an aryl group, a heteroaryl group, an alkoxy group, an aryloxy group, a heteroaryloxy group, an amino group, a cyano group, a halogen atom, a hydroxy group, or a group represented by the following General Formula (6):
[Formula 4]
—R^{P4}—P(═O)R^{P5}—R^{P6} (6)
wherein, in Formula (6), R^{P4} is a single bond, an alkylene group, an arylene group, or a heteroarylene group, and R^{P5} and R^{P6} are each independently an alkyl group, an aryl group, or a heteroaryl group.

3. The metal complex according to claim 1, wherein the R^{A1} to R^{A9}, the R^{C1} to R^{C8}, and the R^{E1} to R^{E6} are each independently a single bond, an alkylene group having 1 to 4 carbon atoms, a phenylene group, a naphthylene group, a pyridylene group, a bipyridylene group, a pyrimidinylene group, or a group represented by the above Formula (4).

4. The metal complex according to claim 1, wherein the R^{B1} to R^{B9}, the R^{D1} to R^{D8}, and the R^{F1} to R^{F6} are each independently a hydrogen atom or a phenanthrolinyl group.

5. The metal complex according to claim 1, wherein the metal complex is any selected from the group consisting of compounds represented by the following L101-M to L108-M, L201-M to L-212-M, and L301-M to L320-M:

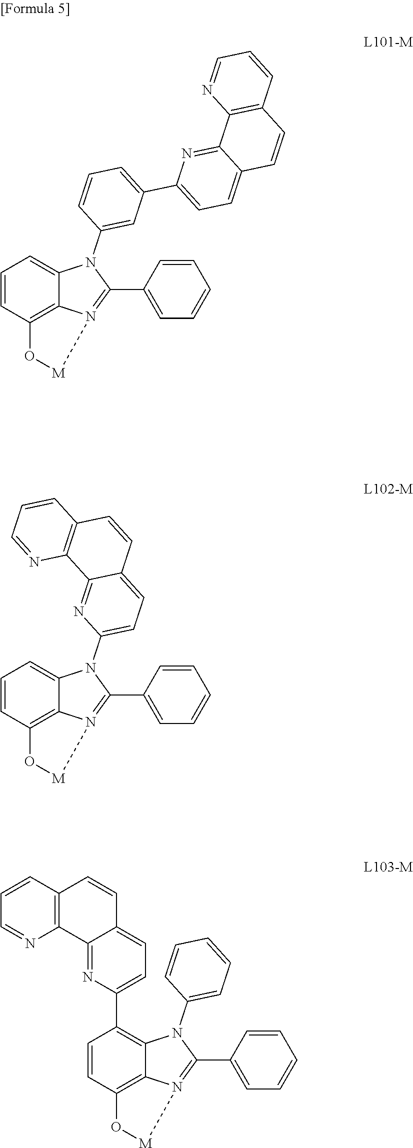

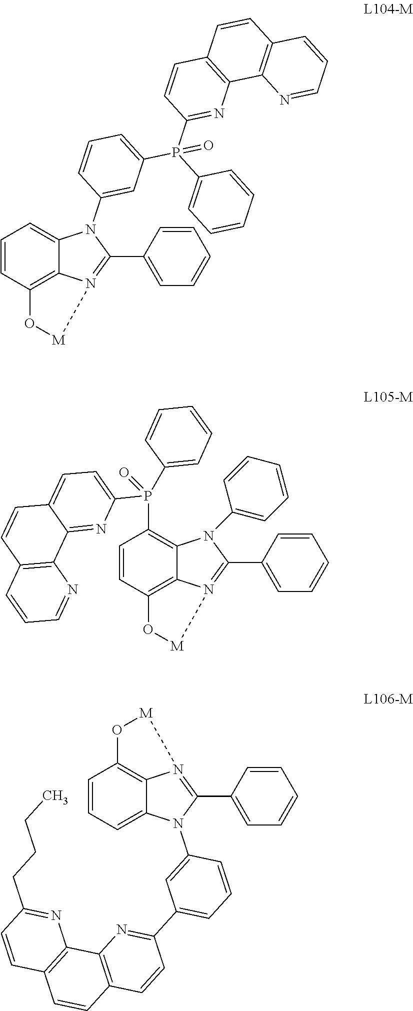

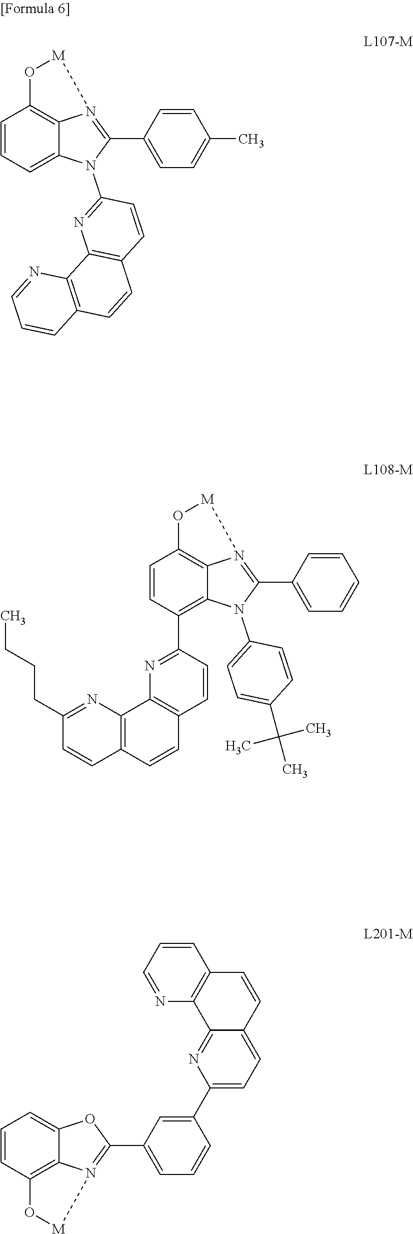

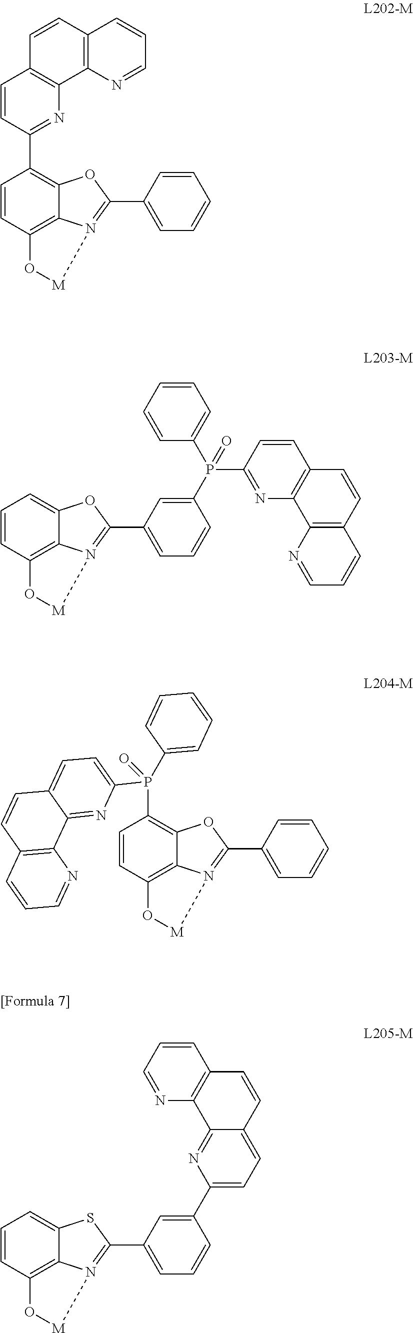

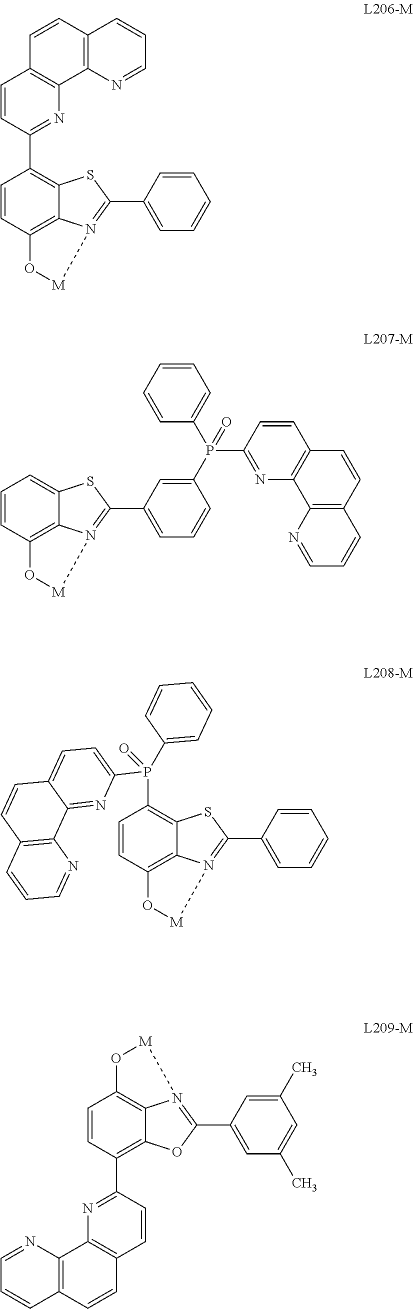

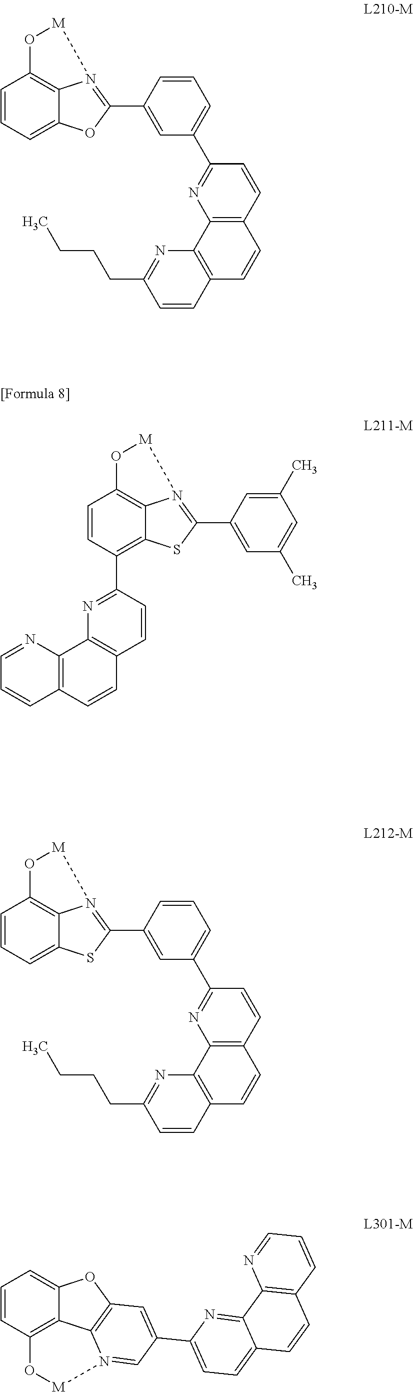

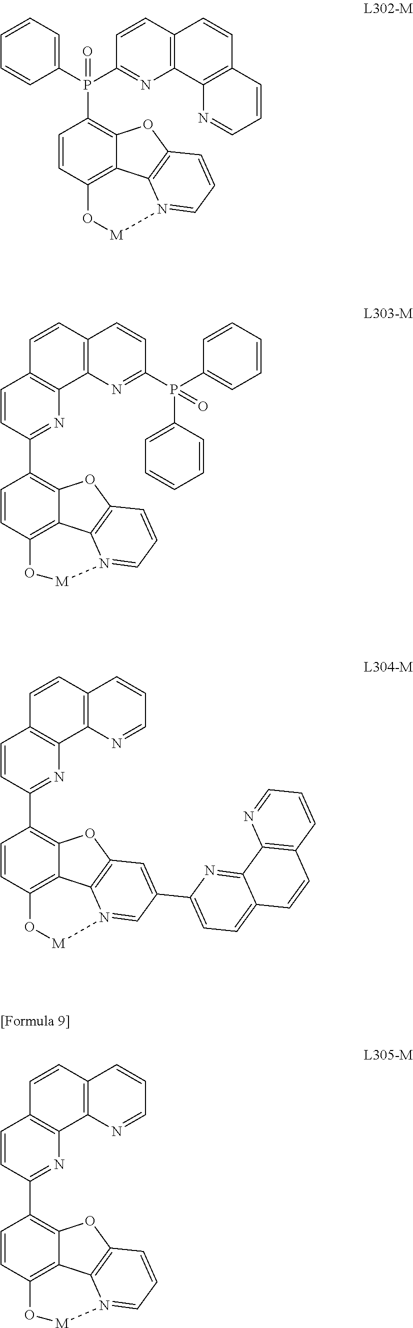

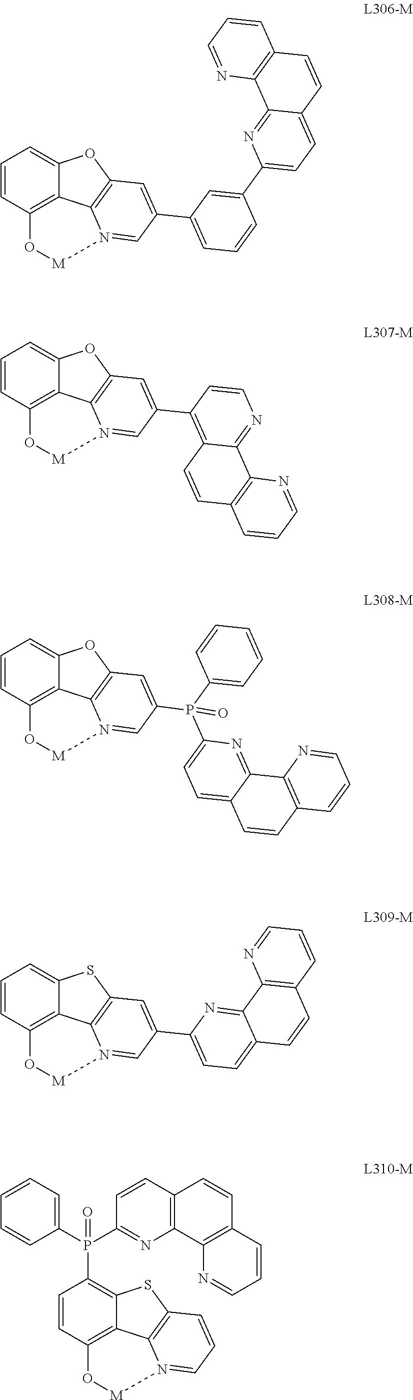

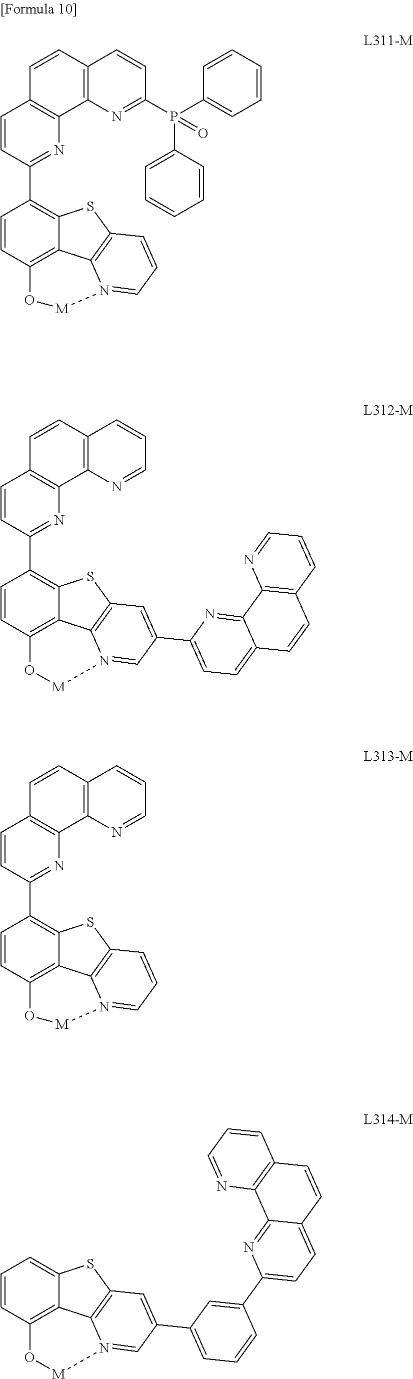

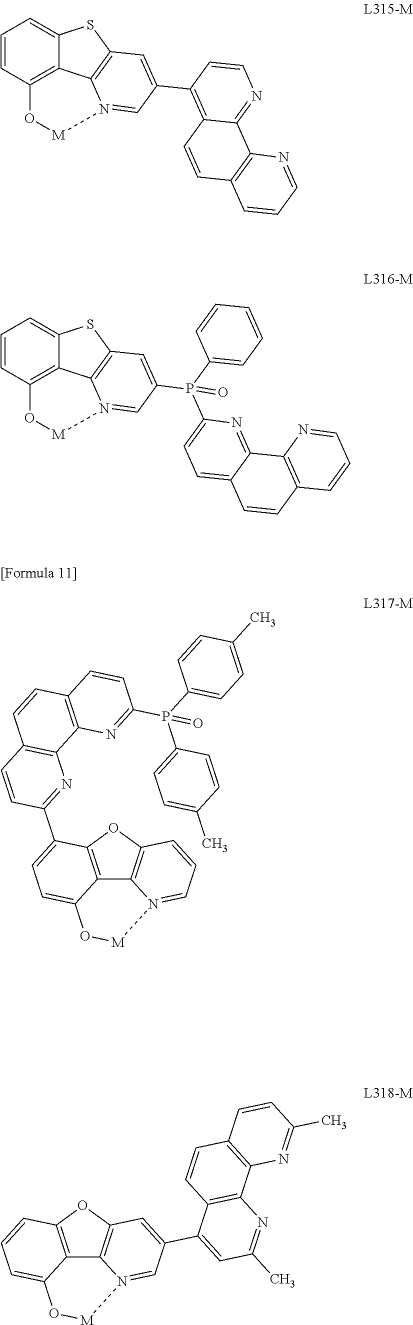

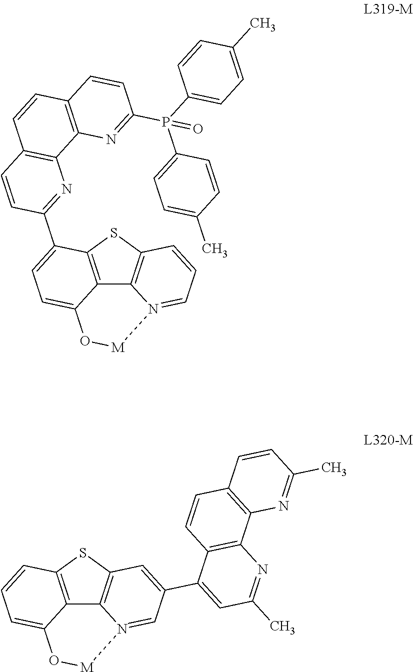

6. The metal complex according to claim 1, wherein the M is an alkali metal.

7. The metal complex according to claim 6, wherein the alkali metal is Rb or Cs.

8. A coordination compound for use in the metal complex according to claim 1.

9. An electron transporting material for an organic electroluminescent element, comprising the metal complex according to claim 1.

10. The electron transporting material according to claim 9, further comprising a dopant.

11. The electron transporting material according to claim 10, wherein the dopant contains a metal alkoxide represented by the following Formula (7a) and/or the following Formula (7b):
[Formula 12]
R^{H1}—O-M (7a)
R^{H1}—O-M²-O—R^{H2} (7b)
wherein in Formula (7a) and Formula (7b), R^{H1} and R^{H2} each independently represent an alkyl group, M¹ is an alkali metal, and M² represents an alkaline earth metal.

12. The electron transporting material according to claim 10, wherein the dopant contains one or more selected from the group consisting of an alkali metal complex of quinolinolate, an alkali metal complex of pyridylphenolate, an alkali metal complex of bipyridylphenolate, and an alkali metal complex of isoquinolinylphenolate.

13. The electron transporting material according to claim 10, wherein the dopant contains one or more selected from the group consisting of an alkali metal hydroxide, an alkali metal halide, an alkali metal carbonate, an alkali metal hydrogen carbonate, an organic acid salt of an alkali metal having 1 to 9 carbon atoms, an alkaline earth metal hydroxide, an alkaline earth metal halide, an alkaline earth metal carbonate, an alkaline earth metal hydrogen carbonate, and an organic acid salt of an alkaline earth metal having 1 to 9 carbon atoms.

14. The electron transporting material according to claim 9, further comprising a ligand constituting the metal complex.

15. A liquid material for constructing an electron transport layer of an organic electroluminescent element, the liquid material comprising the electron transporting material according to claim 9 and a protic polar solvent.

16. The liquid material according to claim 15, wherein the protic polar solvent is an alcoholic solvent having 1 to 12 carbon atoms.

17. The liquid material according to claim 16, wherein the alcoholic solvent having 1 to 12 carbon atoms is a monohydric or dihydric alcohol.

18. The liquid material according to claim 15, comprising 0.01 to 10% by weight of the metal complex.

19. An organic electroluminescent element comprising an electron transport layer containing the electron transporting material according to claim 9.

20. A method for producing an organic electroluminescent element, comprising a step of constructing an electron transport layer of an organic electroluminescent element by a wet process using the liquid material according to claim 15.
